SEQUENCE LISTING (4 sequences)

SEQ ID NO: 1 - protein, 8 aa, synthetic construct
SIINFEKL

SEQ ID NO: 2 - protein, 14 aa, synthetic construct
LEEKKGNYVVTDHC

SEQ ID NO: 3 - protein, 9 aa, synthetic construct
KVPRNQDWL

SEQ ID NO: 4 - protein, 8 aa, synthetic construct
RGYVYQGL

(19) United States
(12) Patent Application Publication
(10) Pub. No.: US 2026/0284192 A1
(43) Pub. Date: Sep. 24, 2026

(54) METHODS AND MATERIALS FOR TREATING CANCER

(71) Applicant: Mayo Foundation for Medical Education and Research, Rochester, MN (US)

(72) Inventors: Timothy J. Kottke, Oronoco, MN (US); Laura Evgin, Vancouver (CA); Richard G. Vile, Rochester, MN (US)

(21) Appl. No.: 19/474,230

(22) PCT Filed: Apr. 11, 2024
(86) PCT No.: PCT/US2024/024062

§ 371 Date: Oct. 10, 2025

Related U.S. Application Data
(60) Provisional application No. 63/458,549, filed on Apr. 11, 2023.

(51) Int. Cl. A61K 40/31; A61K 39/00; A61K 39/395; A61K 40/11; A61K 40/32; A61K 40/42; A61P 35/00

(52) U.S. Cl. CPC A61K 40/31; A61K 39/0011; A61K 39/3955; A61K 40/11; A61K 40/32; A61K 40/4208; A61K 40/4211; A61K 40/4212; A61K 40/4217; A61K 40/4221; A61K 40/4224; A61K 40/4257; A61K 40/4261; A61K 40/4271; A61P 35/00; A61K 2039/5256

(57) ABSTRACT

This document relates to methods and materials involved in treating cancer. For example, methods and materials for using (a) T cells (e.g., chimeric antigen receptor (CAR) T cells), (b) one or more antigenic compositions (e.g., one or more compositions including one or more antigens), and (c) one or more immune checkpoint inhibitors to treat a mammal (e.g., a human) having cancer are provided.

### CROSS-REFERENCE TO RELATED APPLICATIONS

This application claims the benefit of U.S. Patent Application Ser. No. 63/458,549, filed on Apr. 11, 2023. The disclosure of the prior application is considered part of, and is incorporated by reference in, the disclosure of this application.

### SEQUENCE LISTING

This application contains a Sequence Listing that has been submitted electronically as an XML file named “07039-2218WO1.xml.” The XML file, created on Apr. 1, 2024, is 5000 bytes in size. The material in the XML file is hereby incorporated by reference in its entirety.

### TECHNICAL FIELD

This document relates to methods and materials involved in treating cancer. For example, this document provides methods and materials for using (a) T cells (e.g., chimeric antigen receptor (CAR) T cells), (b) one or more antigenic compositions (e.g., one or more compositions including one or more antigens), and (c) one or more immune checkpoint inhibitors for treating a mammal (e.g., a human) having cancer (e.g., cancers that exhibit little or no response to treatment with either CAR T cell therapy or immune checkpoint inhibitors alone).

### BACKGROUND

The rather modest efficacy of chimeric antigen receptor (CAR) T cells against solid tumors derives from multiple immune suppressive mechanisms in the tumor microenvironment, which restrict CAR T cell infiltration, persistence, and function (Newick et al., Annu. Rev. Med., 68:139-152 (2017); Schmidts et al., Front. Immunol., 9:2593 (2018); Morgan et al., Front. Immunol., 9:2493 (2018); and Labanieh et al., Nat. Biomed. Eng., 2 (6): 377-391 (2018)).

### SUMMARY

This document provides methods and materials involved in treating cancer. For example, this document provides methods and materials for using (a) T cells (e.g., CAR⁺ T cells), (b) one or more antigenic compositions (e.g., one or more compositions including one or more antigens), and (c) one or more immune checkpoint inhibitors to treat a mammal (e.g., a human) having cancer. In some cases, a mammal (e.g., a human such as a human having cancer) can be administered (a) a population of different T cells engineered to each include a CAR, (b) an antigenic composition (e.g., to stimulate in vivo generation of memory T cells specific for one or more of the antigen(s) of the antigenic composition via the endogenous T cell receptors (TCRs), with at least some of those generated memory T cells also expressing the CAR), and (c) one or more immune checkpoint inhibitors (e.g., to delay the onset of exhaustion in or another immunosuppressive effect on the generated memory T cells). For example, a mammal can be administered (a) a population of different T cells that each express a particular CAR in addition to their endogenous TCR, (b) an antigenic composition to stimulate in vivo generation of memory T cells from at least a few CAR⁺ T cells of that administered population, and (c) one or more immune checkpoint inhibitors to delay the onset of exhaustion in or another immunosuppressive effect on the generated memory T cells. It will be appreciated that the mammal's natural T cell population (endogenous T cells) will include some members of the repertoire that also will be stimulated via the antigenic composition, but those cells will only include the endogenous TCR and not the CAR. Those memory T cells generated from the population of CAR⁺ T cells administered to the mammal can have the ability to direct an immune response (e.g., expand to form populations of effector T cells) against a target via either the CAR or the endogenous TCR (e.g., an endogenous TCR specific for an antigen of the antigenic composition) of that memory T cell. These generated memory T cells within the mammal can be quickly and effectively stimulated to generate populations of effector T cells that target the targets of the CAR by subsequently administering one or more of the antigens recognized by the endogenous TCR of those CAR⁺ memory T cells.

Co-administration of (a) a population of CAR⁺ T cells each with its own endogenous TCR and (b) an oncolytic virus to a mammal can stimulate in vivo generation of dual-specific tissue-resident memory (T_{RM}) T cells (e.g., T_{RM} CAR⁺ T cells) that can recognize either the target of the CAR (e.g., a cancer cell) via the CAR or a T cell epitope of the oncolytic virus via an endogenous TCR specific for that T cell epitope. In some cases, the dual-specific T_{RM} CAR⁺ T cells generated within a mammal can be reactivated to direct immune responses (e.g., populations of effector T cells) against the target of the CAR (e.g., cancer) by administering a boost of the oncolytic virus (or an antigenic portion thereof) to the mammal. In such cases, the boost can stimulate the memory T cells via their endogenous TCR that is specific for the oncolytic virus, and they can be free to hunt and kill and/or to generate effector T cells that can hunt and kill the CAR targets via their provided CAR.

As demonstrated herein, co-administration of (a) a population of different T cells engineered to each include a CAR, (b) an antigenic composition, and (c) one or more immune checkpoint inhibitors can stimulate in vivo generation of dual-specific tissue-resident memory (T_{RM}) T cells (e.g., T_{RM} CAR⁺ T cells) that (i) can recognize either the target of the CAR (e.g., a cancer cell) via the CAR or a T cell epitope of the oncolytic virus via an endogenous TCR specific for that T cell epitope and (ii) have delayed onset of exhaustion or another immunosuppressive effect. In such cases, dual-specific, T_{RM} CAR T cells generated within a mammal can be persist longer in vivo, and therefore can demonstrate enhanced efficacy of CAR T cell function and antitumor activity. For example, dual-specific, T_{RM} CAR T cells generated within a mammal can have the ability to renew even in the continued presence of antigen.

The ability to generate memory T cells and/or effector T cells in a mammal as described herein (e.g., by administering (a) CAR T cells, (b) one or more antigenic compositions, and (c) one or more immune checkpoint inhibitors) provides a unique opportunity to use immunotherapy to target (e.g., to locate and destroy) cancer cells, including cancer cells in solid tumors, which can be undetectable by the immune system, and cancer cells at secondary (e.g., metastatic) locations. For example, the dual-specific memory T cells can be more active against cancer cells, can persist longer in vivo than conventional CAR⁺ T cells used in current immunotherapies, and can be rapidly re-activated in vivo to generate CAR⁺ effector T cells via a subsequent administration of a boosting antigen, thereby resulting in long-term tumor control.

In general, one aspect of this document features methods for treating a mammal having cancer. The methods can include, or consist essentially of, (a) administering a population of T cells with different endogenous TCRs to a mammal having cancer, where the T cells comprise a CAR that can target the cancer; (b) administering a first antigenic composition to the mammal, where at least some of the T cells of the population form memory T cells within the mammal, where the memory T cells comprise the CAR and an endogenous TCR specific for an antigen of the first antigenic composition; (c) administering a second antigenic composition including the antigen to the mammal, where the memory T cells are stimulated via their endogenous TCRs to form effector T cells comprising the CAR, and where the effector T cells reduce the number of cancer cells within the mammal; and (d) administering an immune checkpoint inhibitor to the mammal. The mammal can be a human. The cancer can be a brain stem glioma, a pancreatic cancer, a bile duct cancer, a lung cancer, a skin cancer, a prostate cancer, a breast cancer, an ovarian cancer, a liver cancer, a colorectal cancer, a germ cell tumor, a hepatocellular carcinoma, a bowel cancer, a multiple myeloma, a lymphoma, or a leukemia. The population of T cells with different endogenous TCRs can include naïve T cells. The naïve T cells can be CD4⁺ T cells, CD8⁺ T cells, or any combination thereof. The CAR can target a tumor-specific antigen on the cancer. The tumor-specific antigen can be cluster of differentiation 19 (CD19), CD22, CD20, GD2, EGFRvIII, mesothelin, IL-13RA, BCMA, CD138, NKG2-D, HER2/Neu, IL-13RA2, CD137, CD28, B7-H3 (CD276), CD16V, CA-125, MUC-1, epithelial tumor antigen, melanoma-associated antigen, mutated p53, mutated Ras, ERBB2, folate binding protein, HIV-1 envelope glycoprotein gp120, HIV-1 envelope glycoprotein gp41, CD123, CD23, CD30, CD56, c-Met, GD3, HERV-K, IL-11R alpha, kappa chain, lambda chain, CSPG4, or VEGFR2. The first antigenic composition can include a virus. The virus can be an oncolytic virus. The virus can be a vesiculovirus, a Maraba virus, a reovirus, an adenovirus, a vaccinia virus, a Newcastle disease virus, a poliovirus, a HSV virus, or a measles virus. The endogenous TCR specific for the antigen can be an endogenous TCR specific for an antigen of the virus. The first antigenic composition can include a virus expressing an antigen exogenous to the virus. The endogenous TCR specific for the antigen can be an endogenous TCR specific for the antigen exogenous to the virus. The first antigenic composition can include an antigenic polypeptide.

The endogenous TCR specific for the antigen can be an endogenous TCR specific for the antigenic polypeptide. The population of T cells with different endogenous TCRs and the first antigenic composition can be administered to the mammal within from about 1 second to about 48 hours of each other. The population of T cells with different endogenous TCRs and the first antigenic composition can be administered to the mammal at the same time. The population of T cells with different endogenous TCRs and the first antigenic composition can be administered to the mammal are as a single composition. The the memory T cells can be CD69⁺ and CD103⁺. The memory T cells can be central memory T cells (T_{CM} cells), effector memory T cells (T_{EM} cells), terminally differentiated effector memory T cells (T_{EMRA} cells), or tissue resident memory T cells (T_{RM} cells). The second antigenic composition can be administered to the mammal at least 5 days after the administering of the population of T cells and the administering of the first antigenic composition. The immune checkpoint inhibitor can be an anti-PD-1 antibody, an anti-PD-L1 antibody, an anti-CTL4A antibody, an anti-LAG-3 antibody, an anti-TIM-3 antibody, or an anti-CD200AR antibody. The immune checkpoint inhibitor can be pembrolizumab, nivolumab, cemiplimab, ipilimumab, tremelimumab, durvalumab, dostarlimab, avelumab, atezolizumab, or relatlimab. The immune checkpoint inhibitor can be BMS-8, BMS-37, BMS-202, BMS-230, BMS-242, BMS-1001, BMS-1166, SB415286, vorinostat, decitabine, entitostat, JQ1, BET151, GSK503, panobinostat, ACY-241, azacytidine, DB36, DB71, DB15, CVN, MGCD0103, SNDX-275, IMP32, BMS986016, TSR-022, Sym023, ATIK2a, or DZNep. The second antigenic composition and the immune checkpoint inhibitor can be administered to the mammal within from about 1 second to about 48 hours of each other. The second antigenic composition and the immune checkpoint inhibitor can be administered to the mammal at the same time. The second antigenic composition and the immune checkpoint inhibitor can be administered to the mammal are as a single composition. The cancer cells within the mammal can be reduced by at least 25 percent. The method can be effective to improve survival of the mammal. The survival of the mammal can be improved by at least 25 percent.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1A-1H. CAR T cells with TCR reactivity to a VSV immunodominant epitope expand following infection. (FIG. 1A) Mice bearing subcutaneous B16EGFRvIII tumors were lymphodepleted on day 3 with 5 gray (Gy) total body radiation, treated on day 4 with 10⁷ EGFRvIII CAR T cells, and on day 9 with PBS or with VSV-mIFNγ intravenously (10⁷ pfu) or intratumorally (IT) (5×10⁷ pfu). (FIG. 1B) On day 16, CD8⁺ CAR T cells (identified by the Thy1.1 marker from the CAR vector) with TCR reactivity to the VSV N₅₂₋₅₉ H-2K^{b} immunodominant epitope were enumerated in the spleen and tumor. n=4 mice per group. Representative flow plots for the CD8⁺ gate are shown for individual mice from each treatment. (FIG. 1C) The composition of the CD8 compartment for individual mice is represented in the inner rings and the average group composition in the outer ring. (FIG. 1D) The percent CD8 CAR T tetramer-positive populations from (FIG. 1A) is represented as mean±SD. In separate cohorts of mice, blood was collected by submandibular vein bleeds on days 17 and 32 and dual-specific CAR T cells were enumerated. n=3 to 10 mice per group. P values were determined using a one-way ANOVA with a Tukey multiple-comparison posttest. (FIG. 1E) Interim blood and experimental endpoint spleens from two independent tumor-cured mice were stained for CD8, Thy1.1, and the VSV N₅₂₋₅₉ H-2K^{b} tetramer. Flow plots show the CD8⁺ Thy1.1⁺ gate. (FIG. 1F) TCR VB distribution as measured by flow cytometry of the CD8 CAR T cells prepared for injection, and the CD8 CAR T recovered from mice on day 16 that also received PBS or VSV-mIFNβ intravenously or intratumorally as in (FIG. 1A). n=4 mice per group. (FIG. 1G) Comparison of the TCRvβ chain usage between the input CAR T cells adoptively transferred into mice and those recovered from recipient mice reveals positive and negative selection for particular chains. (FIG. 1H) Comparison between TCRvβ chain usage in the endogenous CD8 population versus the CD8 CAR T cell population for each mouse. Dashed lines indicate the percent VB chain usage in the input CD8 CAR T cell population. For (FIG. 1D), (FIG. 1G), and (FIG. 1H), each symbol represents a mouse.

FIGS. 2A-2G. Dual-specific CAR T cells have improved function against B16EGFRvIII target cells and acquire a distinct memory phenotype. (FIG. 2A) Mice bearing subcutaneous B16EGFRvIII tumors were lymphodepleted on day 3 with 5 Gy total body radiation, treated on day 4 with 10⁷ EGFRvIII CAR T cells, and on day 9 with PBS or with VSV-mIFNβ intravenously (IV) (10⁷ pfu). On day 16, Thy1.1⁺ CD4⁻ cells were sorted by FACS. (FIG. 2B) Sorted cells were cocultured with CTV-labeled B16EGFRvIII target cells and carboxyfluorescein diacetate succinimidyl ester (CFSE)-labeled B16 nontarget cells in a ratio of 2:1:1 (effector:target:nontarget). Representative flow plots gated on live Thy1.1⁻ cells are shown on the left. In the right panel, the percent specific killing of target cells is represented as the means±SD. The P value was determined using an unpaired two-tailed t test. (FIG. 2C) Splenocytes from (FIG. 2A) were left unstimulated or stimulated with the VSV N_{52 59} peptide. Sorted Thy1.1⁺ CD4⁻ cells were cocultured with B16 parental or B16EGFRvIII cells at an effector:target:non-target ratio of 2:1:1 for 6 hours in the presence of brefeldin A and monensin. Cells were stained with CD107a during the coculture and the VSV N tetramer and for cytokine production following the coculture. Representative flow plots show degranulation (CD107a) and cytokine (IFNγ) production. (FIG. 2D) The full intracellular cytokine panel is shown for all mice stimulated as described in (FIG. 2C). Lines connect the unstimulated and stimulated conditions for each animal. n=4 mice per group. (FIG. 2E) Group means of Boolean gating demonstrating coexpression of cytokines and degranulation as in (FIG. 2D). (FIG. 2F) CD8 CAR T cells from the spleens of mice treated as in (FIG. 2A) were assayed for expression of KLRG1, CD127, and CD62L. Representative flow plots gated on CD8⁺ Thy1.1⁺ cells and are indicated as tetramer positive or negative. (FIG. 2G) Percent expression of CD62L, CD127, and KLRG1 as in (FIG. 2F) is represented as the means±SD. n=4 to 6 mice per group. P values were determined using a one-way ANOVA with a Tukey multiple-comparison posttest. For (FIG. 2B), (FIG. 2D), and (FIG. 2G), each symbol represents a mouse.

FIGS. 3A-3F. In vitro virus loading promotes dual specific (DS) CAR T cell generation with improved therapeutic efficacy. (FIG. 3A) Mice bearing subcutaneous B16EGFRvIII tumors were treated with 10⁷ pfu of VSV-GFP IV or with 10⁷ EGFRvIII CAR T cells loaded with VSV-GFP (MOI 1) at 4° C. for 1 hour (CAR (VSV) IV) on day 10. Two days later, lymph nodes (LNs) and tumors were harvested and dissociated, and virus was titered on BHK cells. n=3 mice per group. The P value was determined using an unpaired two-tailed t test on log-transformed data. (FIG. 3B) Mice bearing subcutaneous B16EGFRvIII tumors were treated with 10⁷ EGFRvIII CAR T cells either left unloaded or loaded with VSV-GFP (MOI 1), LPS, polyI:C, CpG, or reovirus (MOI 1) at 4° C. for 1 hour. Two days later, LNs and tumors were harvested and dissociated, and the number of Thy 1.1⁺ CD8⁺ CAR T cells was enumerated. n=3 mice per group. P values were determined using a one-way ANOVA with a Tukey multiple-comparison posttest using log-transformed data. (FIG. 3C) Non-tumor-bearing mice were treated with 10⁷ EGFRvIII CAR T cells either unloaded or loaded with VSV-GFP (MOI 1) at 4° C. for 1 hour (CAR (VSV) IV). A third group treated with unloaded CAR T cells was subsequently treated with 10⁷ pfu of VSV-GFP 5 days later. Two weeks after adoptive cell transfer, Thy1.1⁺CAR T cells were isolated from spleens, and 10⁵ cells were cocultured with B16EGFRvIII or B16 cells pretreated for 24 hours with IFNγ or with murine in vitro matured dendritic cells preloaded for 24 hours with VSV-N₅₂₋₅₉ or OVA-derived SIINFEKL (SEQ ID NO:1 peptide at an E:T ratio of 10:1. Forty-eight hours later, IFNγ was measured by ELISA. P values were determined using a one-way ANOVA with a Tukey multiple-comparison posttest using log-transformed data. (FIG. 3D and FIG. 3E) Mice bearing subcutaneous B16EGFRvIII tumors were treated on day 8 with either PBS, 10⁷ CAR T cells, 10⁷ pfu of VSV-mIFNβ, or 10⁷ CAR T cells loaded with VSV-mIFNβ (MOI 1) at 4° C. for 1 hour (CAR (VSV)) intravenously. On day 15, mice were given an intravenous boost with PBS, 10⁷ pfu of VSV-mIFNβ, or 10⁸ pfu of a replication-incompetent adenovirus Ad-OVA. Survival of mice is shown. n=7 to 8 mice per group. A log-rank Mantel-Cox test was performed with Bonferroni multiple-comparison correction; statistical significance was set at P=0.0083. (FIG. 3F) Thy1.1⁺CAR T cells were isolated from spleens of mice in the experiment of (FIG. 3E) either at the time of euthanasia due to tumor size or at the end of the experiment (day 60). Cells (2.5×10⁵) were cocultured with live B16EGFRvIII or B16 cells pretreated for 24 hours with IFNγ or with murine in vitro matured dendritic cells preloaded for 24 hours with VSV-N₅₂₋₅₉ or OVA-derived SIINFEKL (SEQ ID NO:1) peptide at an E:T ratio of 10:1. Forty-eight hours later, IFNγ was measured by ELISA. n=3 mice per group. P values were determined using a one-way ANOVA with a Tukey multiple-comparison posttest. For (FIG. 3A), (FIG. 3B), (FIG. 3C), and (FIG. 3F), the group mean is represented±SD. Each symbol represents a mouse.

FIGS. 4A-4I. Reovirus-loaded CAR T cells are therapeutic in multiple tumor models. (FIG. 4A and FIG. 4B) Mice bearing subcutaneous B16EGFRvIII tumors were treated on day 7 with either PBS, 10⁷ pfu of reovirus, or 10⁷ CAR T cells (left unloaded or loaded in vitro with reovirus (MOI 1.0, 4° C., 1 hour)). On day 15, mice were boosted with PBS or 10⁷ pfu of VSV-GFP or reovirus. Survival of mice is shown. n=7 mice per group. A log-rank Mantel-Cox test was performed with Bonferroni multiple-comparison correction; statistical significance was set at P=0.017. (FIG. 4C) Splenocytes from mice treated in (FIG. 4B) at euthanasia were pooled (n=3 mice per group), and flow plots gated on the CD8⁺ population show the percent Thy1.1⁺. (FIG. 4D and FIG. 4E) Mice bearing brainstem CT2AEGFRvIII tumors were treated on day 7 with PBS, 10⁷ pfu of reovirus, or 10⁷ CAR T cells (left unloaded or loaded in vitro with reovirus (MOI 1.0, 4° C., 1 hour)). On day 15, mice were boosted with PBS or 10⁷ pfu of VSV-GFP or reovirus. A log-rank Mantel-Cox test was performed with Bonferroni multiple-comparison correction; statistical significance was set at P=0.0125. (FIG. 4F) A separate cohort of mice treated as in (FIG. 4D) (n=3 mice per group) were euthanized on day 25, and the brains were harvested. Flow plots gated on the CD8⁺ population show the percent Thy1.1⁺ from the pooled groups. (FIG. 4G) Splenocytes from (FIG. 4E) were cocultured with CT2AEGFRvIII cells or CT2A parental cells at an effector-to-target ratio of 5:1 for 48 hours. IFNγ secretion was measured by ELISA. n=3 mice per group. P values were determined using a one-way ANOVA with a Tukey multiple-comparison posttest using log-transformed data. The group mean is represented±SD. Each symbol represents a mouse. (FIG. 4H and FIG. 4I) Mice bearing frontal lobe CT2AEGFRvIII tumors were treated on day 8 with PBS, 10⁷ pfu of reovirus, or 10⁷ CAR T cells. These CAR T cells were prepared from either naïve C57BL/6 mice (CAR_{NAIVE}) or from reovirus-immune mice vaccinated 3 weeks previously with 10⁸ pfu of reovirus (CAR_{IMMUNE}). CAR T_{NAIVE} or CAR T_{IMMUNE} were left unloaded (CAR_{NAIVE} or CAR_{IMMUNE}) or loaded in vitro with reovirus (MOI 1.0, 4° C., 1 hour) (CAR_{NAIVE}(Reo) or CAR_{IMMUNE}(Reo)). On day 15, mice were boosted with PBS (/PBS) or 10⁷ pfu of reovirus (/boost). Survival of mice is shown. n=10 mice per group. A log-rank Mantel-Cox test was performed with Bonferroni multiple-comparison correction; statistical significance was set at P=0.0071.

FIGS. 5A-5F. Dual-specific CAR T cells can be expanded with TCR specificity for virus-encoded antigens. (FIG. 5A and FIG. 5B) Mice bearing subcutaneous B16EGFRvIII tumors were treated on day 8 with either PBS, 10⁸ pfu of Ad-OVA, or 10⁷ CAR T cells (left unloaded or loaded in vitro with Ad-OVA (MOI 1) at 4° C. for 1 hour (CAR (Ad-OVA))). On day 15, mice were boosted with PBS or 10⁷ pfu of Ad-GFP, VSV-GFP, or VSV-OVA. Survival of mice is shown. n=7 to 8 mice per group. A log-rank Mantel-Cox test was performed with Bonferroni multiple-comparison correction; statistical significance was set at P=0.017. (FIG. 5C) Splenocytes from representative animals were isolated from mice in the experiment of (FIG. 5B) either at the time of euthanasia due to tumor size or at the end of the experiment (day 62). Flow plots gated on the CD8⁺ population show the percent Thy1.1⁺ and SIINFEKL (SEQ ID NO:1) tetramer⁺ populations. (FIG. 5D and FIG. 5E) Mice bearing subcutaneous B16EGFRvIII tumors were treated on day 8 with either PBS, 10⁷ CAR T cells, 10⁷ pfu of VSV-hGP100, or 10⁷ CAR T cells loaded with VSV-hGP100 (MOI 1) at 4° C. for 1 hour (CAR (VSV-hGP100)). On day 15, mice were given an intravenous boost with PBS, 10⁸ pfu of Ad-GFP, or Ad-hGP100. n=7 to 8 mice per group. A log-rank Mantel-Cox test was performed with Bonferroni multiple-comparison correction; statistical significance was set a P=0.017. (FIG. 5F) At the time of euthanasia due to tumor size or at the conclusion of the experiment (d95), Thy1.1⁺CAR T cells were isolated from spleens, and 10⁵ cells were cocultured with B16EGFRvIII or B16 cells pretreated for 24 hours with IFγ or with murine in vitro matured dendritic cells preloaded for 24 hours with the VSV N₅₂₋₅₉ H-2K^{b}-restricted peptide or the OVA-derived SIINFEKL (SEQ ID NO:1) peptide (5 μg/mL) at an E:T ratio of 10:1. Forty-eight hours later, IFNγ was measured by ELISA. P values were determined using a one-way ANOVA with a Tukey multiple-comparison posttest using log-transformed data. The group mean is represented ±SD. Each symbol represents a mouse.

FIGS. 6A-6C. Combination therapy can be effective against TAA_{CAR}-loss tumors. (FIG. 6A and FIG. 6B) Mice bearing brainstem tumors composed entirely of CT2AEGFRvIII cells or 10% CT2AEGFRvIII+90% CT2A cells were treated on day 8 with intravenous PBS, 10⁷ pfu of reovirus, 10⁷ CAR T cells, or 10⁷ CAR T cells loaded with reovirus (MOI 1) at 4° C. for 1 hour (CAR (Reo)). On day 15, mice received a systemic boost with PBS, 10⁷ pfu of VSV-GFP, or 10⁷ pfu of reovirus. n=7 to 8 mice per group. A log-rank Mantel-Cox test was performed with Bonferroni multiple-comparison correction; statistical significance was set at P=0.017. (FIG. 6C) Splenocytes were recovered from mice at the time of euthanasia due to tumor size or at the end of the experiment of (FIG. 6B). Splenocyte cells (10⁶) were cocultured with CT2A or B16EGFRvIII cells pretreated for 24 hours with IFNγ, at an E:T ratio of 10:1. Forty-eight hours later, IFNγ was measured by ELISA. P values were determined using a one-way ANOVA with a Tukey multiple-comparison posttest using log-transformed data. The group mean is represented±SD. Each symbol represents a single mouse.

FIGS. 7A-7D. In vitro expansion and functional characterization of human DS CAR T cells. (FIG. 7A) Experimental setup for (FIG. 7B) (black dashed boxes) and (FIG. 7C) (blue dashed boxes). (FIG. 7B) Human anti-CD19 CAR T cells from three separate donors were left unloaded or were loaded in vitro at MOI 1.0, 1 hour, 4° C. with reovirus. A total of 10⁶ CAR T or CAR (Reo) T cells were cocultured with autologous CD14⁺ APCs at a ratio of 10 CAR: 1 CD14⁺ cell. Two, 5, and 8 days later, 10⁵ additional autologous CD14⁺ APCs were added to the cultures. On day 10, CD3⁺ T cells were reisolated by magnetic bead sorting, and 10⁶ T cells were cocultured with IFNγ-pretreated parental Mel888 cells, Mel888-CD19 cells, reovirus-infected Mel888 cells (MOI 0.1), or VSV-infected Me1888 cells (MOI 0.001) at an E:T ratio of 10:1. Twenty-four hours later, IFNγ secreted into the supernatant was measured by ELISA. P values were calculated using a two-way repeated-measures ANOVA with a Sidak multiple-comparison test. The group mean is represented±SD. Each symbol represents a donor and connected samples from the same donor. (FIG. 7C) Untransduced activated T cells or CD19 CAR-transduced T cells from three separate healthy human donors were left unloaded or loaded in vitro at MOI 1.0, with nothing, VSV-TYRP1, VSV-GFP, or LPS (1 hour, 4° C.). A total of 10⁶ UTD, CAR T, or loaded CAR T cells were cocultured with autologous CD14⁺APCs at a ratio of 10 CAR: 1 CD14⁺ cell. Two, 5, and 8 days later, 10⁵ additional autologous CD14⁺APC were added to the cultures. On day 10, CD3⁺ T cells were reisolated by magnetic bead sorting, and 10⁶ T cells were cocultured with IFNγ-pretreated parental Hep3B, Mel888 cells, Raji, or Mel888-CD19 cells at an E:T ratio of 10:1 in ELISpot wells. Forty-eight hours later, wells were developed, and the number of spots was counted. P values were calculated using a two-way repeated-measures ANOVA with a Tukey multiple-comparison test. (FIG. 7D) NSG mice bearing MEL888-CD19 subcutaneous tumors were treated intravenously with PBS, 10⁷ human anti-CD19 CAR T cells (CAR), 10⁷ human anti-CD19 CAR T cells loaded in vitro with reovirus (4° C., 1 hour, MOI 10) (CAR (Reo)), or 10⁷ human activated CD⁸+ UTD T cells loaded in vitro with reovirus (4° C., 1 hour, MOI 10) (UTD (Reo)). Tumor size with time is shown. In the CAR (Reo)-treated group, three mice in which complete tumor regression had occurred were euthanized owing to the development of GVHD toxicity at days 47, 54, and 61.

FIG. 8. Enumeration of CAR T cells in the tumor, spleen and blood using the reverse order schedule. Mice bearing subcutaneous B16EGFRvIII tumors were lymphodepleted on day 3 with 5 Gy total body radiation, treated on day 4 with 10⁷ EGFRvIII CAR T cells and on day 9 with PBS or with VSV-mIFNβ intravenously (IV) (10⁷ pfu) or intratumorally (IT) (5×10⁷ pfu). On day 16, mice were euthanized and the skin or receded subcutaneous tumor, and spleen were isolated to enumerate CD8⁺CAR T cells by flow cytometry. In a separate cohort of mice, blood was collected on day 17 and CD8⁺ CAR T cells were quantified by flow cytometry. Each symbol represents a mouse. n=4-15 mice/group. P values were determined using a one-way ANOVA with a Tukey multiple comparisons post-test.

FIGS. 9A-9B. Flow cytometry verification of CAR expression in transduced T cells, and sample gating scheme. (FIG. 9A) Concordance of the Thy1.1 marker and a tetramer specific for the EGFRvIII CAR. Splenocytes were left untransduced (UTD) or transduced with the MSGV1 retroviral vector encoding the EGFRvIII CAR and the Thy1.1 marker from an IRES. To confirm the specificity of the Thy 1.1 marker for transduced CAR expressing cells, a home-made tetramer reagent was used (PepvIII SA-AF647). The tetramer was composed of a biotin labeled peptide sequence (LEEKKGNYVVTDHC; SEQ ID NO:2) from EGFRvIII that is recognized by the scFv in the CAR and labeled with streptavidin (SA)-Alexa fluor 647. The plots for the UTD cells (red) were overlaid on the CAR cells (blue). (FIG. 9B) Sample gating schemes for CD8 CAR T and VSV N₅₂₋₅₉ tetramer staining in splenocytes and subcutaneous tumors treated as in FIGS. 1 and 2.

FIGS. 10A-10C. Expansion of virus specific CAR T cells in non-lymphodepleted mice. (FIG. 10A) Mice bearing subcutaneous B16EGFRvIII tumors were treated on day 4 with 10⁷ EGFRvIII CAR T cells and on day 9 with PBS or with VSV-mIFNβ intravenously (IV) or intratumorally (IT) (5×10⁷ pfu). On day 16, CD8⁺ endogenous and CAR T cells (identified by Thy1.1 expression) with TCR reactivity to the VSV N₅₂₋₅₉ H-2K^{b} immunodominant epitope were enumerated in the spleen and tumor. n=4 mice/group. (FIG. 10B) Representative flow plots for the CD8+ Thy1.1+ (CAR) or Thy1.1⁻ (endogenous) gates are shown for individual mice from each treatment. (FIG. 10C) The percent CD8 CAR T tetramer positive populations is represented as mean±SD. P values were determined using an unpaired two-tailed T test. Each symbol represents a mouse.

FIGS. 11A-11C. Expansion of DS CAR T with multiple reactivities from naïve mice. (FIG. 11A) Splenocytes were isolated from spleens of naïve C57BL/6 mice or from mice vaccinated with 10⁶ pfu VSV-IFNβ 3 weeks previously. 10⁵ cells were co-cultured with no added peptide or with 1 μg/mL of VSV N₅₂₋₅₉, OVA-derived SIINFEKL (SEQ ID NO: 1), or H-2 Db restricted human GP10025-33 (KVPRNQDWL; SEQ ID NO:3) peptide in ELISPOT wells. 48 hours later wells were developed and the number of spots counted. Each symbol represents a mouse. n=3 mice/group. (FIG. 11B) EGFRvIII CAR T cells prepared from either naïve C57BL/6 mice or from mice vaccinated with 10⁶ pfu VSV-IFNβ 3 weeks previously, were co-cultured with murine in vitro matured dendritic cells pre-loaded for 24 hours with no peptide or with 5 μg/mL of VSV N₅₂₋₅₉, OVA-derived SIINFEKL (SEQ ID NO: 1), or H-2 Db restricted human GP10025-33 (KVPRNQDWL; SEQ ID NO:3) peptide in ELISPOT wells at an Effector:Target ratio of 10:1. 48 hours later wells were developed and the number of spots counted. Limit of quantitation was 500 spots. Each symbol represents a mouse. n=3 mice/group. P values were determined using a one-way ANOVA with a Tukey multiple comparisons post-test. (FIG. 11C) Mice were implanted with B16EGFRvIII tumors subcutaneously, irradiated on day 3, and treated with 10⁷ CAR T on day 4. Select groups received either PBS, or 5×10⁷ pfu VSV-mIFNβ, VSVhGP100 or VSVOVA IT on day 9. Spleens were harvested on day 16 and stained with the VSV N₅₂₋₅₉ tetramer. Flow plots are gated on live CD8⁺ Thy1.1⁺ cells. n=3 or 4 mice per group.

FIGS. 12A-12B. Expression of KLRG1, CD127 and CD62L on endogenous CD8 populations. Mice bearing subcutaneous B16EGFRvIII tumors were lymphodepleted on day 3 with 5 Gy total body radiation, treated on day 4 with 10⁷ EGFRvIII CAR T cells and on day 9 with PBS or with VSV-mIFNβ intravenously (IV) (10⁷ pfu). Splenocytes were harvested on day 16. (FIG. 12A) CD8 endogenous (Thy1.1⁻) expression of KLRG1, CD127 and CD62L is represented as group means±SD. Each symbol represents a mouse. n=4 mice/group. (FIG. 12B) Group means of Boolean gating demonstrating average co-expression of markers.

FIGS. 13A-13C. In vivo expansion of endogenous and CAR T cells with reactivity to virus or virus encoded antigens. (FIG. 13A) Thy1.1⁻ CD8⁻ T cells were isolated from spleens of mice treated as in FIG. 3D and euthanized on day 21. 2.5×10⁵ cells were co-cultured with B16EGFRvIII or B16 cells pre-treated for 24 hours with IFNγ; or with murine in vitro matured dendritic cells pre-loaded for 24 hours with VSV N₅₂₋₅₉ or OVA-derived SIINFEKL (SEQ ID NO:1) peptide at an E:T ratio of 10:1. 48 hours later, the concentration of IFNγ was measured by ELISA as shown. n=3 mice/group. (FIG. 13B) Thy1.1 CD8⁺ T cells were isolated from spleens of mice treated as in FIG. 5D and euthanized on day 21·10⁵ cells were co-cultured with B16EGFRvIII or B16 cells pre-treated for 24 hours with IFNγ, or with murine in vitro matured dendritic cells pre-loaded for 24 hours with the VSV N52-59 peptide or the OVA-derived SIINFEKL (SEQ ID NO:1) peptide (5 μg/mL) at an E:T ratio of 10:1. 48 hours later, the concentration of IFNγ was measured by ELISA as shown. n=3 mice/group. (FIG. 13C) On day 1, C57BL/6 mice were treated with either 10⁷ CAR T cells or with 10⁷ CAR T cells loaded with VSV-hGP100 (MOI 1) at 4° C. for 1 hour (CAR(VSV-hGP100)). On day 8, mice were given an IV boost with PBS, 10⁸ pfu Ad-GFP or Ad-hGP100. On day 15 Thy1.1⁺ (CAR T) or Thy1.1 (non-CAR T) CD8⁺ T cells were isolated from spleens and 10⁵ cells were co-cultured with murine in vitro matured dendritic cells pre-loaded for 24 hours with the hGP10025-33 H-2 Db restricted peptide (5 μg/mL) at an E:T ratio of 10:1. 48 hours later, the concentration of IFNγ was measured by ELISA. P values were determined using a one-way ANOVA with a Tukey multiple comparisons post-test using log-transformed data. n=3 mice/group. The group mean is represented as ±SD. Each symbol represents a mouse.

FIGS. 14A-14C. Transfer of virus from CAR T cells to tumor cells. (FIG. 14A) Human CD19 CAR T cells from 3 separate donors were loaded in vitro at MOI 0.01, 0.1, 1.0, 10 or 100 for 1 hour at 4° C. with reovirus. 10⁶ CAR (Reo) T cells were then co-cultured with either Mel888 or Mel888-CD19 target cells at a ratio of 0.1 CAR: 1 target cell in order to minimize target cell killing by CAR T. 72 hours later reovirus released into the supernatant was collected and titered on L929 cells. (FIG. 14B) 10⁷ EGFRvIII CAR T cells were loaded with reovirus or VSV at 4° C. for 1 hour at an MOI of 1.0. CAR T cells were pelleted, re-suspended in 1 mL PBS and then pelleted again. Virus titer in the PBS was measured (Wash 1); this was repeated twice (Wash 2 and Wash 3). (FIG. 14C) Following the final pelleting CAR T cells were re-suspended in CAR medium and allowed to expand in vitro. 48 and 72 hours later, viral titers (combined cell lysate and supernatants) were measured on BHK (VSV) and L292 (reovirus) cells. n=3 technical replicates.

FIG. 15. At day 60 following tumor implantation, spleens of mice which survived tumors following treatment with either CAR T (VSV-ova)/Ad-OVA Boost/Control IgG with SIINFEKL (SEQ ID NO:1) or with CAR T (VSV-ova)/Ad-OVA Boost/α-PD-1 were analyzed for the presence of CAR T cells (Q1), anti-OVA CD8⁺ T cells (Q3) or dual specific CAR/anti-OVA CD8 T cells (Q2) by flow cytometry.

### DETAILED DESCRIPTION

This document provides methods and materials involved in treating cancer. For example, this document provides methods and materials for using (a) T cells (e.g., CAR⁺ T cells), (b) one or more antigenic compositions (e.g., one or more compositions including one or more antigens), and (c) one or more (e.g., one, two, three, four, or more) immune checkpoint inhibitors to treat a mammal (e.g., a human) having cancer (e.g., a cancer that exhibits little or no response to treatment with either CAR T cell therapy or immune checkpoint inhibitors alone). In some cases, a mammal (e.g., a human such as a human having cancer) can be administered (a) a population of different T cells engineered to each include a CAR, (b) an antigenic composition (e.g., a composition including one or more viruses such as one or more oncolytic viruses, a composition including one or more viruses designed to express one or more antigens of interest, and/or a composition including one or more antigenic polypeptides of interest), and (c) one or more immune checkpoint inhibitors to stimulate in vivo generation of memory T cells that (i) are specific for one or more of the antigen(s) of the antigenic composition via the endogenous TCRs, with at least some of those generated memory T cells also expressing the CAR, and (ii) have delayed the onset of exhaustion or another immunosuppressive effect. For example, a mammal can be administered (a) a population of different T cells that each express a particular CAR in addition to their endogenous TCR, (b) an antigenic composition, and (c) one or more immune checkpoint inhibitors to stimulate in vivo generation of memory T cells from at least a few CAR⁺ T cells of that administered population. It will be appreciated that the mammal's natural T cell population (endogenous T cells) will include some members of the repertoire that also will be stimulated via the antigenic composition, but those cells will only include the endogenous TCR and not the CAR. On the other hand, those memory T cells generated from the population of CAR⁺ T cells administered to the mammal can have the ability to direct an immune response (e.g., generate a population of CAR⁺ effector T cells) against a target via either the CAR or the endogenous TCR (e.g., an endogenous TCR specific for an antigen of the antigenic composition) of that memory T cell.

In some cases, administering (a) a population of different T cells engineered to each include a CAR, (b) an antigenic composition (e.g., a composition including one or more viruses such as one or more oncolytic viruses, a composition including one or more viruses designed to express one or more antigens of interest, and/or a composition including one or more antigenic polypeptides of interest), and (c) one or more immune checkpoint inhibitors to mammal can be effective to stimulate a cell-mediated immune response within the mammal. For example, CAR⁺ T cells within the population that are administered to a mammal together with an antigenic composition and that have an endogenous TCR specific for an antigen within that antigenic composition can be stimulated within the mammal via that endogenous TCR and the presence of the antigen. Once stimulated, the CAR⁺ T cells can mediate an immune response against the targets of the CAR. In some cases, administering (a) a population of different T cells engineered to each include a CAR, (b) an antigenic composition, and (c) one or more immune checkpoint inhibitors to a mammal can be effective to convert naïve CAR⁺ T cells administered to the mammal into memory T cells that are less susceptible to T cell exhaustion or another immunosuppressive effect within the mammal. In some cases, those generated memory T cells can be dual-specific in that they are CAR⁺ and include an endogenous TCR that recognizes an epitope from the antigenic composition. In such cases, subsequent administration of an antigenic composition that includes that epitope can result in those dual-specific memory T cells expanding quickly and effectively to generate a population of CAR⁺ effector T cells that can hunt and kill cells expressing the target of that CAR.

In some cases, memory T cells generated within a mammal (e.g., a human) as described herein (e.g., by administering (a) CAR T cells, (b) one or more antigenic compositions, and (c) one or more immune checkpoint inhibitors) can be less susceptible to T cell exhaustion (e.g., can have a delayed onset of exhaustion) (or another immunosuppressive effects) as compared to memory T cells generated within a mammal (e.g., a human) in the absence of one or more immune checkpoint inhibitors. For example, memory T cells generated within a mammal (e.g., a human) by administering (a) CAR T cells, (b) one or more antigenic compositions, and (c) one or more immune checkpoint inhibitors as described herein can persist longer within the mammal (e.g., as compared to T cells such as CAR T cells that are administered without an antigenic composition, without subsequently administering (e.g., boosting) with an antigenic composition, and/or any immune checkpoint inhibitor). For example, the materials and methods described herein can be used to generate T cells that can persist within a mammal (e.g., a human) for from about 2 years to about 10 years (e.g., from about 2 years to about 8 years, from about 2 years to about 5 years, from about 2 years to about 4 years, from about 2 years to about 3 years, from about 3 years to about 10 years, from about 5 years to about 10 years, from about 8 years to about 10 years, from about 3 years to about 8 years, from about 4 years to about 6 years, from about 3 years to about 5 years, from about 5 years to about 7 years, or from about 6 years to about 8 years).

Any appropriate mammal having cancer (e.g., a cancer including one or more solid tumors) can be treated as described herein (e.g., by administering (a) CAR T cells, (b) one or more antigenic compositions, and (c) one or more immune checkpoint inhibitors). Examples of mammals that can have cancer and can be treated as described herein include, without limitation, humans, non-human primates (e.g., monkeys), horses, bovine species, porcine species, dogs, cats, mice, and rats. In some cases, a human having cancer (e.g., a cancer including one or more solid tumors) can be treated by administering (a) CAR T cells, (b) one or more antigenic compositions, and (c) one or more immune checkpoint inhibitors.

A mammal (e.g., a human) having any type of cancer can be treated as described herein (e.g., by administering (a) CAR T cells, (b) one or more antigenic compositions, and (c) one or more immune checkpoint inhibitors). In some cases, a cancer that can be treated as described herein can include one or more solid tumors. In some cases, a cancer that can be treated as described herein can be a blood cancer. In some cases, a cancer treated as described herein can be resistant to one or more immune checkpoint inhibitors. In some cases, a cancer treated as described herein can be a primary cancer. In some cases, a cancer treated as described herein can be a metastatic cancer. In some cases, a cancer treated as described herein can be a refractory cancer. In some cases, a cancer treated as described herein can express a tumor-specific antigen (e.g., an antigenic substance produced by a cancer cell). Examples of cancers that can be treated as described herein include, without limitation, brain cancers (e.g., brain stem gliomas such as high-grade gliomas (HGGs)), pancreatic cancers (e.g., pancreatic adenocarcinoma), bile duct cancers (e.g., cholangiocarcinoma), lung cancers (e.g., mesothelioma), skin cancers (e.g., melanoma), prostate cancers, breast cancers, ovarian cancers, liver cancers, colorectal cancers, germ cell tumors, hepatocellular carcinoma, bowel cancers, multiple myelomas, lymphomas (e.g., B cell lymphomas such as diffuse large cell lymphoma), leukemias (e.g., chronic lymphocytic leukemia (CLL), acute lymphoblastic leukemia (ALL), and acute myeloid leukemia (AML)), and uveal melanomas. In some cases, a cancer treated as described herein can be a brain stem glioma (e.g., a HGG). For example, a cancer treated as described herein can be a brain stem glioma (e.g., a HGG) in a pediatric human.

In some cases, the methods described herein also can include identifying a mammal as having cancer. Examples of methods for identifying a mammal as having cancer include, without limitation, physical examination, laboratory tests (e.g., blood and/or urine tests for, for example, circulating tumor DNA and/or levels of tumor antigens (e.g., levels of circulating PSA for prostate cancer)), biopsy, imaging tests (e.g., X-ray, PET/CT, MRI, and/or ultrasound), nuclear medicine scans (e.g., bone scans), endoscopy, and/or genetic tests. Once identified as having cancer, a mammal can treated as described herein (e.g., by administering (a) CAR T cells, (b) one or more antigenic compositions, and (c) one or more immune checkpoint inhibitors). For example, a mammal can be administered or instructed to self-administer (a) a population of T cells described herein (e.g., a population of different T cells engineered to each include a CAR) (b) an antigenic composition described herein (e.g., a composition including one or more viruses such as one or more oncolytic viruses, a composition including one or more viruses designed to express one or more antigens of interest, and/or a composition including one or more antigenic polypeptides of interest), and (c) optionally one or more immune checkpoint inhibitors. Then, in some cases, after at least about 5 days (e.g., after at least about 7 days, after at least about 10 days, after at least about 14 days, after at least about 20 days, after at least about 50 days, after at least about 60 days, after at least about 75 days, after at least about 3 months, after at least about 4 months, after at least about 5 months, or after at least about 6 months) after the latter administration of the population of T cells and the antigenic composition, the mammal can be administered or instructed to self-administer (a) a second antigenic composition that includes at least some of the antigens present in the first antigenic composition administered to the mammal and (b) one or more immune checkpoint inhibitors.

A mammal (e.g., a human) having cancer (e.g., a cancer including one or more solid tumors) can be administered, or can be instructed to self-administer, any appropriate population of T cells. A population of different T cells engineered to each include a CAR can include any type(s) of T cells. In some cases, a population of T cells can include two or more (e.g., two, three, four, five, or more) different types of T cells. For example, a population of T cells can be a polyclonal population of T cells (e.g., can include a polyclonal population CAR⁺ T cells). In some cases, a population of T cells can be a population of naïve T cells. In some cases, a population of T cells can be a population of stimulated T cells. Examples of T cells that can be designed to express an antigen receptor (e.g., a CAR) and used as described herein include, without limitation, naive T cells (e.g., CD4⁺ naive T cells and/or CD8⁺ naive T cells), cytotoxic T cells (e.g., CD4⁺ CTLs and/or CD8⁺ CTLs), tissue resident memory T cells, and central memory T cells. In some cases, a population of T cells can be obtained from a mammal (e.g., a mammal having cancer). For example, a population of T cells (e.g., a natural T cell population) can be obtained from a mammal to be treated using the materials and methods described herein. In some cases, a population of T cells can be obtained from a donor mammal (e.g., a donor mammal of the same species) as the mammal to be treated using the materials and methods described herein. For example, when treating a human, a population of T cells can be obtained from a donor human. In some cases, when treating a human, a population of T cells can be obtained from a donor transgenic pig donor that was engineered to be compatible with humans. In cases where a donor mammal and the mammal to be treated using the materials and methods described herein are humans, the donor human and the human to be treated using the materials and methods described herein can present the same or similar human leukocyte antigens (HLAs; e.g., can be HLA-matched).

In some cases, a population of T cells described herein (e.g., a population of different T cells engineered to each include a CAR) can include or be representative of an endogenous TCR repertoire. For example, a population of T cells described herein can include greater than about 10³ (e.g., greater than 10⁴, greater than 10⁵, greater than 10⁶, greater than 10⁷, greater than 10⁸, greater than 10⁹, greater than 10¹⁰, or greater than 10¹¹) different TCRs (e.g., different endogenous TCRs). For example, a population of T cells described herein can include from about 10³ to about 10¹¹ (e.g., about 10⁴ to about 10¹¹, about 10⁵ to about 10¹¹, about 10⁶ to about 10¹¹, about 10⁷ to about 10¹¹, about 10⁸ to about 10¹¹, about 10³ to about 10¹⁰, about 10⁴ to about 10¹⁰, about 10⁵ to about 10¹⁰, about 10⁶ to about 10¹⁰, about 10⁷ to about 10¹⁰, about 10⁸ to about 10¹⁰, about 10⁵ to about 10⁹, about 10⁶ to about 10⁹, about 10⁷ to about 10⁹, about 10⁸ to about 10⁹, about 10⁵ to about 10⁸, about 10⁶ to about 10⁸, or about 10⁷ to about 10⁸) different TCRs (e.g., different endogenous TCRs).

In some cases, a T cell (e.g., a CAR⁺ T cell) in a population of T cells described herein (e.g., a population of different T cells engineered to each include a CAR) can be as described elsewhere (see, e.g., International Patent Application Publication No. WO 2022/125901 at, for example, page 19, line 6 through page 20, line 8).

A T cell (e.g., a CAR⁺ T cell) in a population of T cells described herein (e.g., a population of different T cells engineered to each include a CAR) that can be administered to a mammal (e.g., a human) as described herein can express (e.g., can be engineered to express) any appropriate antigen receptor. In some cases, an antigen receptor can be a heterologous antigen receptor. In some cases, an antigen receptor can be a CAR. In some cases, an antigen receptor can be a tumor antigen (e.g., tumor-specific antigen) receptor. For example, the T cells of a population of T cells can be engineered to express a tumor-specific antigen receptor that targets a tumor-specific antigen (e.g., a cell surface tumor-specific antigen) expressed by a cancer cell in a mammal having cancer. Examples of antigens that can be recognized by an antigen receptor (e.g., a CAR) expressed in a T cell as described herein include, without limitation, cluster of differentiation 19 (CD19), CD22, CD20, GD2, EGFRvIII, mesothelin, IL-13RA, BCMA, CD138, NKG2-D, HER2/Neu, IL-13RA2, CD137, CD28, B7-H3 (CD276), CD16V, CA-125, MUC-1, epithelial tumor antigen, melanoma-associated antigen, mutated p53, mutated Ras, ERBB2, folate binding protein, HIV-1 envelope glycoprotein gp120, HIV-1 envelope glycoprotein gp41, CD123, CD23, CD30, CD56, c-Met, GD3, HERV-K, IL-11R alpha, kappa chain, lambda chain, CSPG4, and VEGFR2.

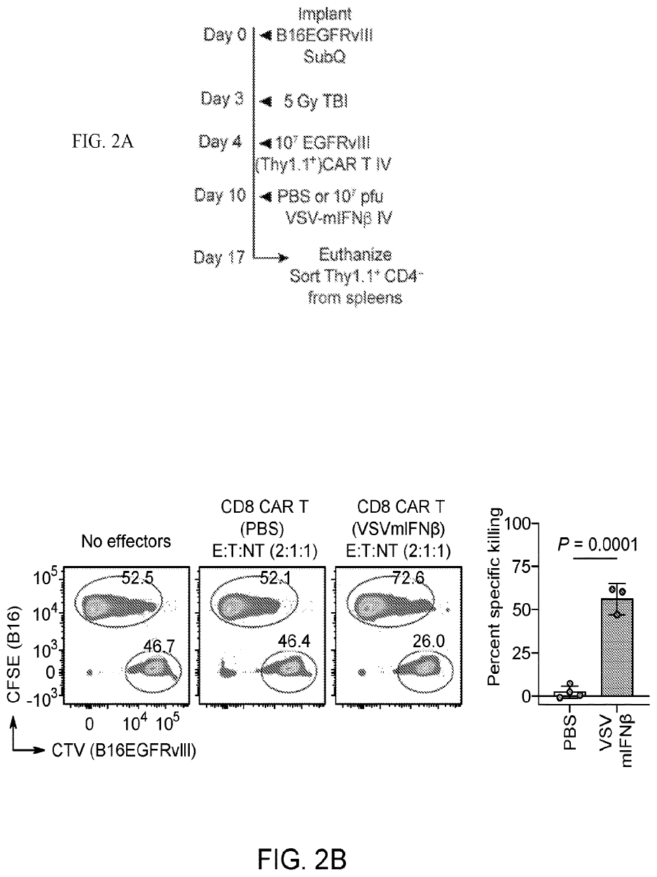

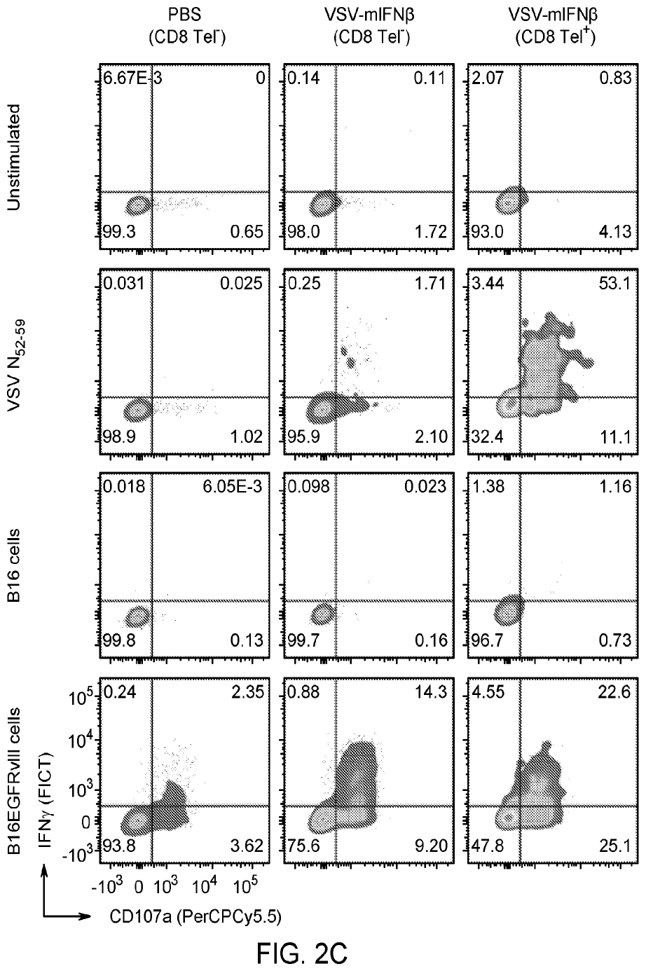

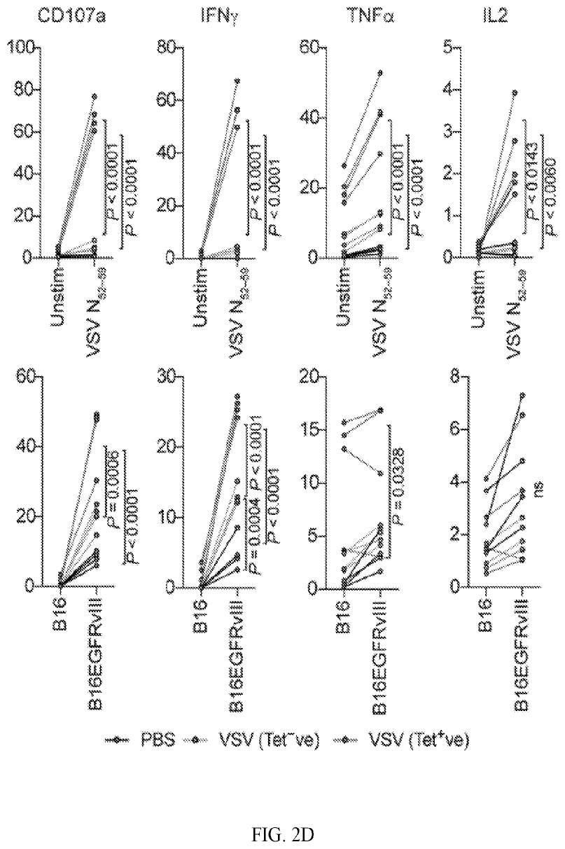

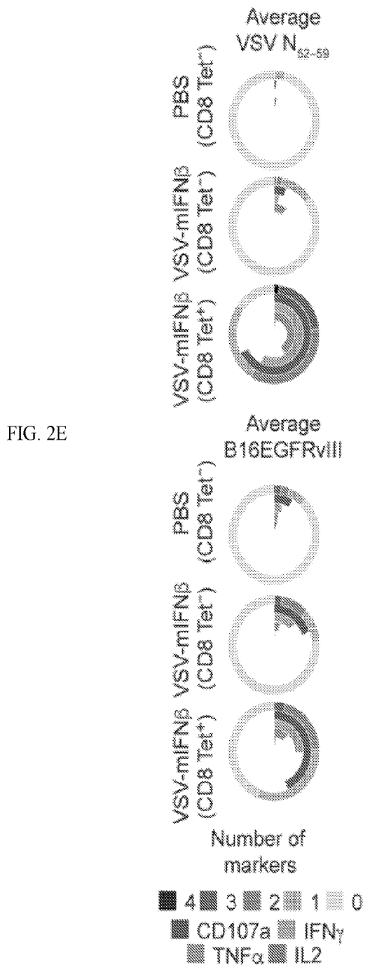

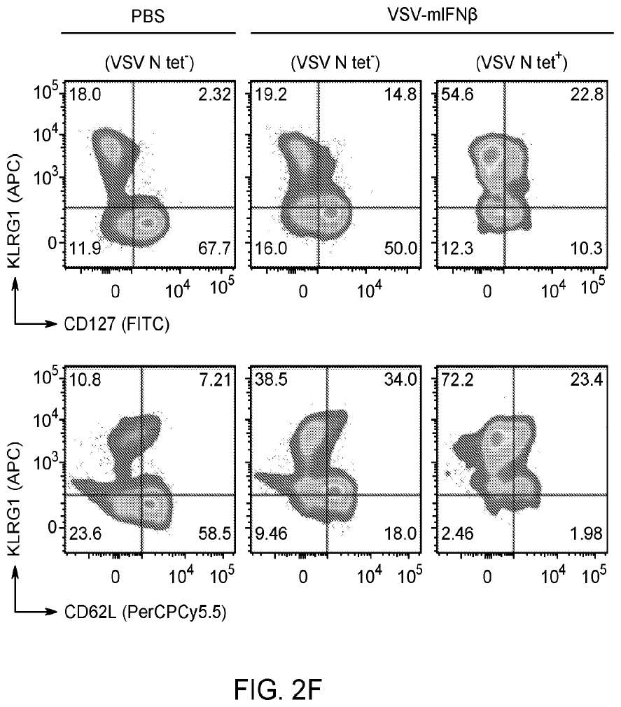

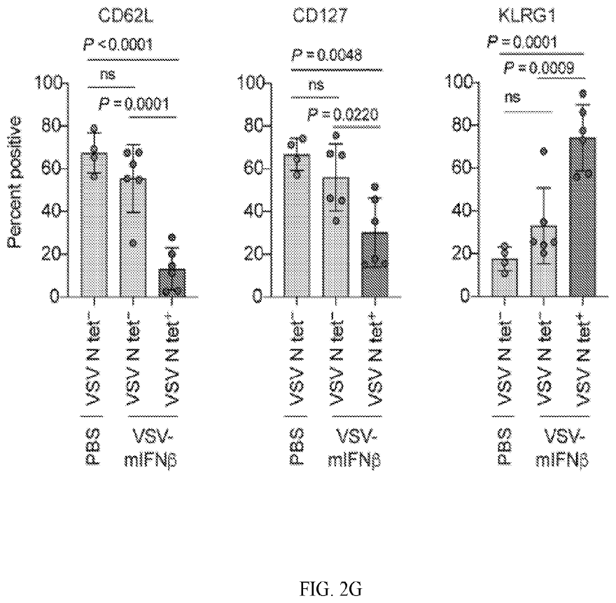

When T cells of a population of T cells described herein (e.g., a population of different T cells engineered to each include a CAR) that can be administered to a mammal (e.g., a human) as described herein are CAR⁺ T cells, the CAR can be any appropriate CAR. A CAR can include an antigen-binding domain, an optional hinge, a transmembrane domain, and one or more signaling domains. An antigen-binding domain of a CAR to be used in a CAR⁺ T cell that can be administered to a mammal (e.g., a human) as described herein can be any appropriate antigen-binding domain. In some cases, an antigen-binding domain can include an antibody or a fragment thereof that targets an antigen (e.g., a cancer antigen such as a CD19 polypeptide). Examples of antigen-binding domains include, without limitation, an antigen-binding fragment (Fab), a variable region of an antibody heavy (VH) chain, a variable region of a light (VL) chain, a single chain variable fragment (scFv), and domains from growth factors that bind to a cancer cell-specific receptor (e.g., domains from EGF, PDGR, FGF, TGF, or derivatives thereof). In some cases, an antigen-binding domain can target (e.g., can target and bind to) a tumor-specific antigen. For example, a CAR⁺ T cell described herein can express (e.g., can be engineered to express) a CAR that can bind to a tumor-specific antigen (e.g., an antigen present on cancer cells with minimal, or no, expression on non-cancerous cell types). In some cases, an antigen-binding domain of a CAR can be as described elsewhere (see, e.g., U.S. Patent Application Publication No. 2017/0183418 such as U.S. Patent Application Publication No. 2017/0183418 at paragraph and the sequence listing; U.S. Patent Application Publication No. 2017/0183413 such as U.S. Patent Application Publication No. 2017/0183413 at paragraph [0049], FIG. 2, Table 9, and the sequence listing; U.S. Patent Application Publication No. 2018/0291079 such as U.S. Patent Application Publication No. 2018/0291079 at paragraphs [0041]-[0045], and Table 4; U.S. Patent Application Publication No. 2020/0289563 such as U.S. Patent Application Publication No. 2020/0289563 at paragraphs [0006]-[0053], [0186]-[0189], and Table 1; and U.S. Patent Application Publication No. 2003/0211097 such as U.S. Patent Application Publication No. 2003/0211097 at paragraphs and [0211-0215] and the sequence listing.

In some cases, a CAR to be used in a CAR⁺ T cell of a population of T cells described herein (e.g., a population of different T cells engineered to each include a CAR) can include an optional hinge region. In some cases, a hinge region can be located between an antigen-binding domain and a transmembrane domain of a CAR. In some cases, a hinge region can provide a CAR with increased flexibility for the antigen-binding domain. For example, a hinge region can reduce spatial limitations of an antigen-binding domain of a CAR and its target antigen (e.g., to increase binding between an antigen-binding domain of a CAR and its target antigen). Examples of hinge regions that can be used as described herein include, without limitation, a membrane-proximal region from an IgG, a membrane-proximal region from CD8, and a membrane-proximal region from CD28. In some cases, a hinge region of a CAR can be as described elsewhere (see, e.g., U.S. Patent Application Publication No. 2018/0000914 such as U.S. Patent Application Publication No. 2018/0000914 at paragraph [0168], and Table 1; U.S. Patent Application Publication No. 2017/0183418 such as U.S. Patent Application Publication No. 2017/0183418 at paragraphs [0034], [0037], [0040], and Table 2; U.S. Patent Application Publication No. 2017/0183413 such as U.S. Patent Application Publication No. 2017/0183413 at paragraph [0116]; and U.S. Patent Application Publication No. 2017/0145094 such as U.S. Patent Application Publication No. 2017/0145094 at paragraph [0104].

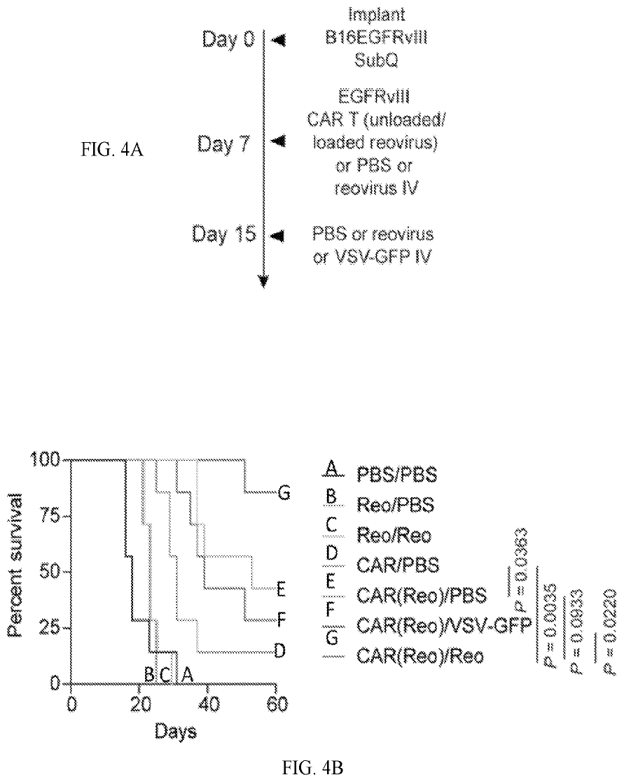

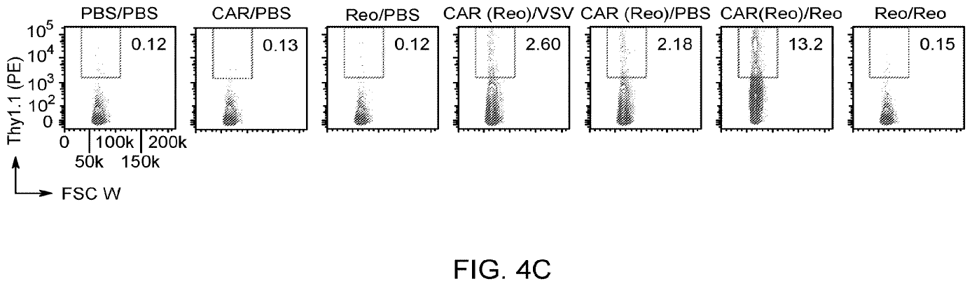

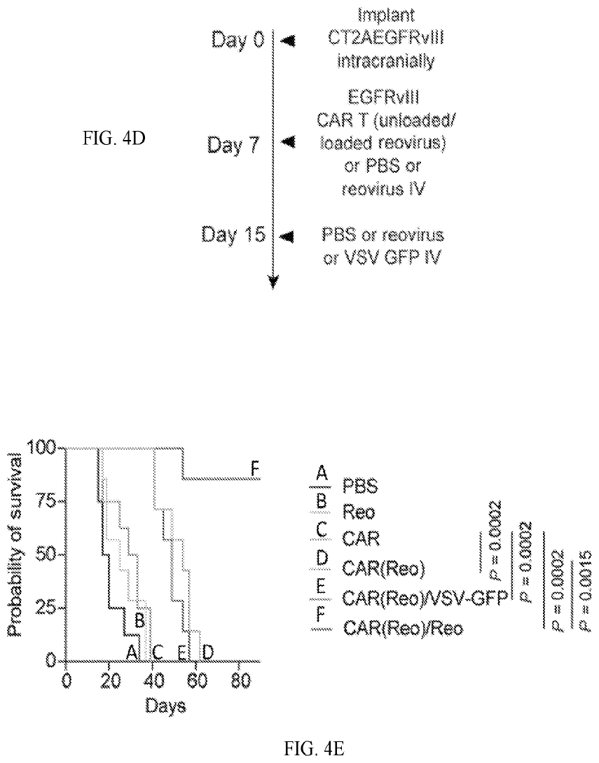

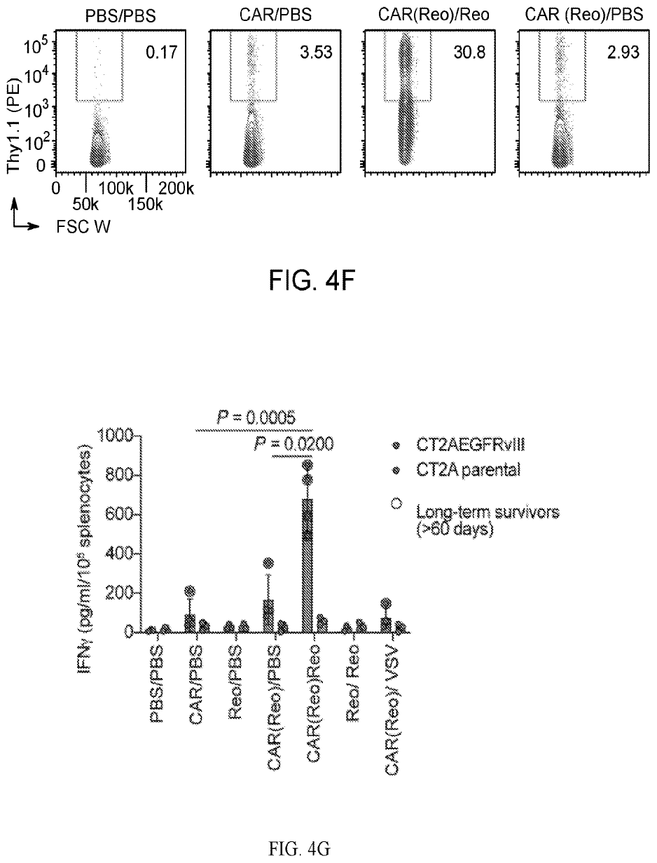

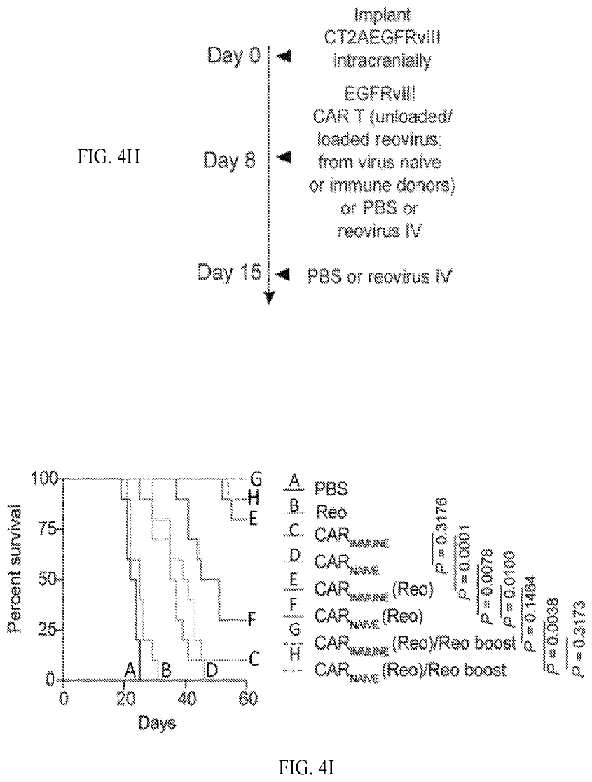

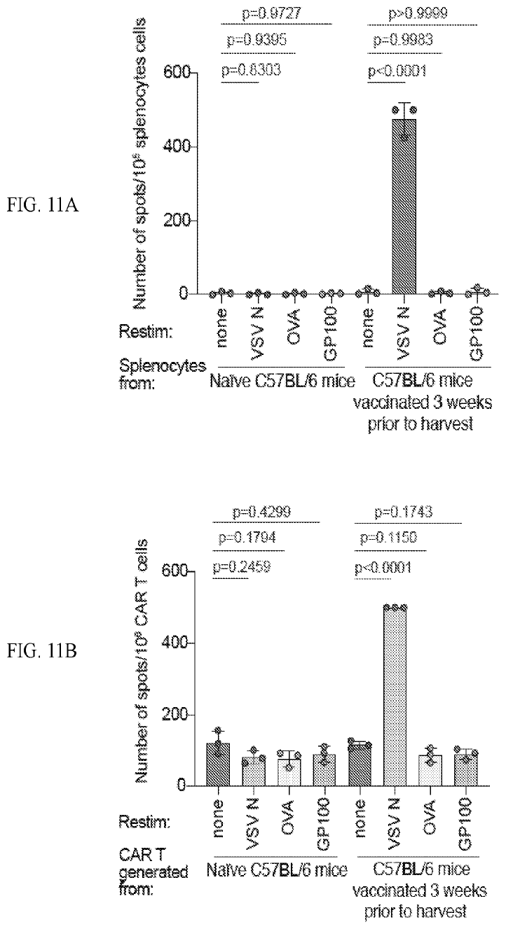

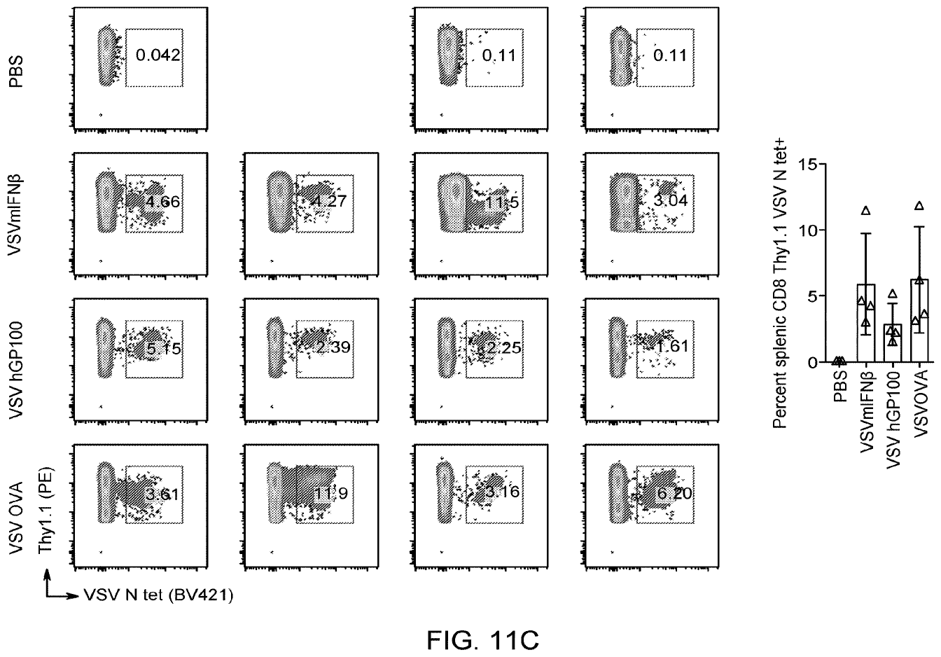

A transmembrane domain of a CAR to be used in a CAR⁺ T cell of a population of T cells described herein (e.g., a population of different T cells engineered to each include a CAR) that can be administered to a mammal (e.g., a human) as described herein can include any appropriate transmembrane domain. A transmembrane domain can be located between an antigen-binding domain and a signaling domain of a CAR and/or located between a hinge and a signaling domain of a CAR. In some cases, a transmembrane domain can provide structural stability for the CAR. For example, a transmembrane domain can include a structure (e.g., a hydrophobic alpha helix structure) that can span a cell membrane and can anchor the CAR to the plasma membrane. Examples of transmembrane domains that can be used as described herein include, without limitation, CD35 transmembrane domains, CD4 transmembrane domains, CD8 (e.g., a CD8a) transmembrane domains, CD28 transmembrane domains, CD16 transmembrane domains, and erythropoietin receptor transmembrane domains. In some cases, a transmembrane domain of a CAR can be as described elsewhere (see, e.g., U.S. Patent Application Publication No. 2016/0120906 such as U.S. Patent Application Publication No. 2016/0120906 at paragraphs [0155], [0161], [0269], FIG. 4, and FIG. 11; U.S. Patent Application Publication No. 2019/0209616 such as U.S. Patent Application Publication No. 2019/0209616 at paragraph [0026]; U.S. Patent Application Publication No. 2018/0000914 such as U.S. Patent Application Publication No. 2018/0000914 at paragraphs [0168]-[0171]; U.S. Patent Application Publication No. 2017/0183418 such as U.S. Patent Application Publication No. 2017/0183418 at paragraphs [0116]-[0118]; U.S. Patent Application Publication No. 2017/0183413 such as U.S. Patent Application Publication No. 2017/0183413 at paragraphs [0116]-[0118]; and U.S. Patent Application Publication No. 2017/0145094 such as U.S. Patent Application Publication No. 2017/0145094 at paragraphs [0104]-[0107].

The signaling domain(s) of a CAR to be used in a CAR⁺ T cell of a population of T cells described herein (e.g., a population of different T cells engineered to each include a CAR) that can be administered to a mammal (e.g., a human) as described herein can include any appropriate signaling domain or combination of signaling domains (e.g., a combination of two, three, or four signaling domains). In some cases, a signaling domain of a CAR can be an intracellular signaling domain normally found within T cells or NK cells. Examples of signaling domains that can be used as described herein include, without limitation, CD2 signaling domains, CD3ζ signaling domains, CD28 signaling domains, Toll-like receptor (TLR) signaling domains (e.g., TLR3 or TLR4 signaling domains), CD27 intracellular signaling domains, OX40 (CD134) intracellular signaling domains, 4-1BB (CD137) intracellular signaling domains, CD278 intracellular signaling domains, DAP10 intracellular signaling domains, DAP12 intracellular signaling domains, FceRly intracellular signaling domains, CD278 intracellular signaling domains, CD122 intracellular signaling domains, CD132 intracellular signaling domains, CD70 intracellular signaling domains, cytokine receptor intracellular signaling domains, and CD40 intracellular signaling domains. In some cases, a CAR for use as described herein can be designed to be a first generation CAR having a CD35 intracellular signaling domain. In some cases, a CAR for use as described herein can be designed to be a second generation CAR having a CD28 intracellular signaling domain followed by a CD35 intracellular signaling domain. In some cases, a CAR for use as described herein can be designed to be a third generation CAR having (a) a CD28 intracellular signaling domain followed by (b) a CD27 intracellular signaling domain, an OX40 intracellular signaling domains, or a 4-1BB intracellular signaling domain followed by (c) a CD3 intracellular signaling domain. In some cases, the intracellular signaling domain(s) of a CAR can be as described elsewhere (see, e.g., U.S. Patent Application Publication No. 2018/0000914 such as U.S. Patent Application Publication No. 2018/0000914 at paragraphs [0164]-[0167]; and U.S. Patent Application Publication No. 2017/0183413 such as U.S. Patent Application Publication No. 2017/0183413 at paragraphs [0122]-[0115].

Examples of CARs that can be expressed on one or more T cells of a population of T cells described herein (e.g., a population of different T cells engineered to each include a CAR) include, without limitation, EGFRvIII CARs, GD2 CARS, IL-13RA CARs, CD19 CARs, BCMA CARs, CD138 CARs, NKG2-D CARS, HER2 CARS, CD137 CARs, and B7-H3 CARs. Exemplary amino acid sequences for such CARs are set forth in FIG. 4.

Any appropriate method can be used to express an antigen receptor (e.g., a CAR) on the surface of a T cell of a population of T cells described herein (e.g., a population of different T cells engineered to each include a CAR) that can be administered to a mammal (e.g., a human) as described herein. For example, a nucleic acid encoding an antigen receptor (e.g., a CAR) can be introduced into one or more T cells of a population of T cells to be administered to a mammal as described herein. In some cases, viral transduction can be used to introduce a nucleic acid encoding an antigen receptor (e.g., a CAR) into a non-dividing a cell. A nucleic acid encoding an antigen receptor (e.g., a CAR) can be introduced in a T cell using any appropriate method. In some cases, a nucleic acid encoding an antigen receptor (e.g., a CAR) can be introduced into a T cell by transduction (e.g., viral transduction using a retroviral vector such as a lentiviral vector) or transfection. In some cases, a nucleic acid encoding an antigen receptor (e.g., a CAR) can be introduced ex vivo into one or more T cells. For example, ex vivo engineering of T cells expressing an antigen receptor (e.g., a CAR) can include transducing isolated T cells with a lentiviral vector encoding an antigen receptor (e.g., a CAR). In cases where T cells are engineered ex vivo to express an antigen receptor (e.g., a CAR), the T cells can be obtained from any appropriate source (e.g., a mammal such as the mammal to be treated or a donor mammal).

Any number of T cells of a population of T cells described herein (e.g., a population of different T cells engineered to each include a CAR) can be engineered to include a CAR (e.g., can be CAR⁺ T cells). In some cases, at least half of the T cells within a population of T cells to be administered to a mammal as described herein can include a CAR (e.g., can be CAR⁺ T cells). In some cases, from about 25 percent to about 100 percent (e.g., from about 25 percent to about 99 percent, from about 25 percent to about 95 percent, from about 25 percent to about 90 percent, from about 50 percent to about 100 percent, from about 50 percent to about 99 percent, from about 50 percent to about 95 percent, from about 50 percent to about 90 percent, about 50 percent to about 85 percent, about 50 percent to about 80 percent, or from about 25 percent to about 75 percent) of the T cells within a population of T cells to be administered to a mammal as described herein can be CAR⁺ T cells. In some cases, 75 percent or less (e.g., 75 percent or less, 70 percent or less, 60 percent or less, 50 percent or less, 40 percent or less, 30 percent or less, 25 percent or less, 20 percent or less, 15 percent or less, 10 percent or less, 5 percent or less, or 2.5 percent or less) of the T cells within a population of T cells to be administered to a mammal as described herein can lack a CAR (e.g., can be CAR⁻ T cells).

In some cases, the CAR⁺ T cells of a population of T cells to be administered to a mammal as described herein (e.g., a population of different T cells engineered to each include a CAR) can be engineered to each include the same CAR.

In some cases, the CAR⁺ T cells in a population of T cells to be administered to a mammal as described herein (e.g., a population of different T cells engineered to each include a CAR) can be engineered so that some of those CAR⁺ T cells of the population express one CAR and others express a different CAR. In some cases, the CAR⁺ T cells in a population of T cells to be administered to a mammal as described herein (e.g., a population of different T cells engineered to each include a CAR) can be engineered so that the population includes T cells expressing a first CAR, T cells expressing a second CAR that is different from the first CAR, and T cells expressing a third CAR that is different from the first and second CARs. In some cases, the CAR⁺ T cells in a population of T cells to be administered to a mammal as described herein (e.g., a population of different T cells engineered to each include a CAR) can be engineered so that the population includes T cells expressing a first CAR, T cells expressing a second CAR that is different from the first CAR, T cells expressing a third CAR that is different from the first and second CARs, and T cells expressing a fourth CAR that is different from the first, second, and third CARs.

In some cases, an individual T cell in a population of T cells described herein (e.g., a population of different T cells engineered to each include a CAR) can be engineered to include two or more (e.g., two, three, four, five, or more) different CARs.

A mammal (e.g., a human) having cancer (e.g., a cancer including one or more solid tumors) can be administered, or can be instructed to self-administer, any appropriate antigenic composition. An antigenic composition (e.g., a composition including one or more viruses such as one or more oncolytic viruses, a composition including one or more viruses designed to express one or more antigens of interest, and/or a composition including one or more antigenic polypeptides of interest) can include any type of antigen(s). For example, an antigen that can be used in an antigenic composition described herein can be a virus. In some cases, a virus that can be used in an antigenic composition described herein can be an oncolytic virus. In some cases, a virus that can be used in an antigenic composition described herein can be an immunogenic virus. In some cases, a virus that can be used in an antigenic composition described herein can be replication competent. In some cases, a virus that can be used in an antigenic composition described herein can be non-pathogenic (e.g., non-pathogenic to a mammal being treated as described herein). For example, a virus that can be used in an antigenic composition described herein can be genetically modified to render it non-pathogenic to a mammal to be treated. In some cases, a virus that can be used in an antigenic composition described herein can infect dividing cells (e.g., can infect only dividing cells). In some cases, a virus that can be used in an antigenic composition described herein can infect non-dividing cells (e.g., can infect only non-dividing cells). In some cases, a virus that can be used in an antigenic composition described herein can infect a cancer cell expressing an antigen targeted by a CAR⁺ T cell administered together with the antigen (e.g., an antigenic composition containing the virus). In some cases, a virus that can be used in an antigenic composition described herein can bud through the endoplasmic reticulum. In some cases, a virus that can be used in an antigenic composition described herein can bind to a cellular receptor (e.g., bind to a cellular receptor to facilitate viral entry into a cell). Examples of viruses that can be used in an antigenic composition described herein include, without limitation, Rhabdoviruses (e.g., vesiculoviruses (VSVs), and Maraba viruses), reoviruses, adenoviruses, vaccinia viruses, Newcastle disease viruses, polioviruses, HSV viruses, measles viruses, and Ad657.

When an antigen used in an antigenic composition described herein is a virus (e.g., an oncolytic virus), the virus can express (e.g., can be designed to express) one or more antigens (e.g., one or more antigens heterologous to that virus). In some cases, an antigen expressed by a virus (e.g., a heterologous antigen) that can be used in an antigenic composition described herein can be a polypeptide. In some cases, an antigen expressed by a virus (e.g., a heterologous antigen) that can be used in an antigenic composition described herein is not endogenous to the mammal being treated as described herein. In some cases, an antigen expressed by a virus (e.g., a heterologous antigen) can be a full-length antigenic polypeptide. In some cases, an antigen expressed by a virus (e.g., a heterologous antigen) can be a fragment of a full-length polypeptide (e.g., provided that the fragment retains an antigenic property within a mammal being treated). In some cases, an antigen expressed by a virus (e.g., a heterologous antigen) can be derived from a full-length polypeptide (e.g., provided that the fragment retains an antigenic property within a mammal being treated). Examples of antigens that can be expressed by a virus (e.g., a heterologous antigen) in an antigenic composition described herein include, without limitation, ovalbumin polypeptides (OVA) and antigenic fragments thereof, TYRP1 polypeptides and antigenic fragments thereof, TYRP2 polypeptides and antigenic fragments thereof, tyrosinase polypeptides and antigenic fragments thereof, CEA polypeptides and antigenic fragments thereof, MART1 polypeptides and antigenic fragments thereof, MART2 polypeptides and antigenic fragments thereof, SARS-COV-2 spike polypeptides and antigenic fragments thereof, VSV-G polypeptides and antigenic fragments thereof, reovirus surface polypeptides and antigenic fragments thereof, adenovirus coat polypeptides and antigenic fragments thereof, CSDE1 polypeptides and antigenic fragments thereof, and superantigen polypeptides (e.g., Streptococcal pyrogenic exotoxins (SPE), Staphylococcal enterotoxins (SE), and enterotoxogenic E. coli (ETEC) enterotoxins) and antigenic fragments thereof.

A virus expressing one or more antigens can be generated using any appropriate method. In some cases, nucleic acid encoding an antigen (e.g., a heterologous antigen) can be introduced into the genome of a virus such that the antigen is expressed. Nucleic acid encoding an antigen (e.g., a heterologous antigen) can be introduced in the genome of a virus using any appropriate method. In some cases, nucleic acid encoding an antigen (e.g., a heterologous antigen) can be introduced into the genome of a virus by homologous recombination techniques, molecular cloning, and gene editing techniques (e.g., the CRISPR-Cas9 System).

In some cases, an antigen that can be used in an antigenic composition can be an antigenic polypeptide. For example, an antigenic polypeptide that can be used in an antigenic composition described herein can be a polypeptide that is not endogenous to the mammal being treated as described herein. In some cases, an antigenic polypeptide used as described herein can be a full-length antigenic polypeptide. In some cases, an antigenic polypeptide used as described herein can be a fragment of a full-length polypeptide (e.g., provided that the fragment retains an antigenic property within the mammal being treated). In some cases, an antigenic polypeptide used as described herein can be derived from a full-length polypeptide (e.g., provided that the fragment retains an antigenic property within the mammal being treated). In some cases, an antigenic polypeptide can be foreign (e.g., exogenous) to a mammal (e.g., a human) to be treated as described herein. In some cases, an antigenic polypeptide used as described herein can be a polypeptide that has no natural counterparts in the mammal (e.g., the human) to be treated as described herein. In some cases, an antigenic polypeptide used as described herein can be a synthetic polypeptide (e.g., a synthetic polypeptide designed to be a potent immunogenic polypeptide). In some cases, an antigenic polypeptide used as described herein can have no natural counterparts in nature. Examples of antigenic polypeptides that can be included in an antigenic composition to be administered to a mammal (e.g., a human) as described herein include, without limitation, ovalbumin polypeptides (OVA) and antigenic fragments thereof, TYRP1 polypeptides and antigenic fragments thereof, TYRP2 polypeptides and antigenic fragments thereof, tyrosinase polypeptides and antigenic fragments thereof, CEA polypeptides and antigenic fragments thereof, MART1 polypeptides and antigenic fragments thereof, MART2 polypeptides and antigenic fragments thereof, SARS-COV-2 spike polypeptides and antigenic fragments thereof, VSV-G polypeptides and antigenic fragments thereof, reovirus surface polypeptides and antigenic fragments thereof, adenovirus coat polypeptides and antigenic fragments thereof, CSDE1 polypeptides and antigenic fragments thereof, and superantigen polypeptides (e.g., Streptococcal pyrogenic exotoxins (SPE), Staphylococcal enterotoxins (SE), and enterotoxogenic E. coli (ETEC) enterotoxins) and antigenic fragments thereof. In some cases, an antigenic composition described herein can contain one or more antigens of interest in the absence of any virus particles.

In some cases, an antigenic composition described herein (e.g., a composition including one or more viruses such as one or more oncolytic viruses, a composition including one or more viruses designed to express one or more antigens of interest, and/or a composition including one or more antigenic polypeptides of interest) can contain one or more antigens other than polypeptides. Examples of antigens other than polypeptides that can be used in a composition described herein include, without limitation, polysaccharides (e.g., type 3 S. pneumoniae polysaccharide (Pn3P) and/or polysaccharides of MUC-1) and lipids.

In some cases, an antigenic composition described herein (e.g., a composition including one or more viruses such as one or more oncolytic viruses, a composition including one or more viruses designed to express one or more antigens of interest, and/or a composition including one or more antigenic polypeptides of interest) can be as described elsewhere (see, e.g., (see, e.g., International Patent Application Publication No. WO 2022/125901 at, for example, page 25, line 28 through page 28, line 31).

A mammal (e.g., a human) having cancer (e.g., a cancer including one or more solid tumors) can be administered, or can be instructed to self-administer, any appropriate immune checkpoint inhibitor. An immune checkpoint inhibitor can inhibit one or more polypeptides involved in an immune checkpoint pathway. Examples of immune checkpoint pathways include, without limitation, PD-1/PD-L1 pathways, PD-1/PD-L2 pathways, CTLA-4 pathways, TRAIL pathways, LAG-3 pathways, TIM-3/Galectin-9 pathways, and CD200/CD200R pathways. An immune checkpoint inhibitor can inhibit any polypeptide involved in an immune checkpoint pathway. Examples of polypeptides involved in an immune checkpoint pathway that can be inhibited by an immune checkpoint inhibitor as described herein include, without limitation, PD-1 polypeptides, PD-L1 polypeptides, CTLA4 polypeptides, LAG-3 polypeptides, TIM-3 polypeptides, and CD200AR polypeptides.

An immune checkpoint inhibitor can inhibit polypeptide activity of a polypeptide involved in an immune checkpoint pathway or can inhibit polypeptide expression of a polypeptide involved in an immune checkpoint pathway. Examples of compounds that can inhibit polypeptide activity of a polypeptide involved in an immune checkpoint pathway include, without limitation, antibodies (e.g., neutralizing antibodies) that target (e.g., target and bind) to a polypeptide involved in an immune checkpoint pathway and small molecules that target (e.g., target and bind) to a polypeptide involved in an immune checkpoint pathway. Examples of compounds that can inhibit polypeptide expression of a polypeptide involved in an immune checkpoint pathway include, without limitation, nucleic acid molecules designed to induce RNA interference of polypeptide expression of a polypeptide involved in an immune checkpoint pathway (e.g., a siRNA molecule or a shRNA molecule), antisense molecules that can target (e.g., are complementary to) nucleic acid encoding a polypeptide involved in an immune checkpoint pathway, and miRNAs that can target (e.g., are complementary to) nucleic acid encoding a polypeptide involved in an immune checkpoint pathway. Examples of immune checkpoint inhibitors that can be administered to mammal (e.g., a human) having cancer (e.g., a cancer including one or more solid tumors) include, without limitation, anti-PD-1 antibodies, anti-PD-L1 antibodies, anti-CTL4A antibodies, anti-LAG-3 antibodies, anti-TIM-3 antibodies, and anti-CD200AR antibodies. In some cases, an immune checkpoint inhibitor that can be administered to mammal (e.g., a human) having cancer (e.g., a cancer including one or more solid tumors) as described herein can be as shown in Table 1.

TABLE 1
Exemplary immune checkpoint inhibitors.

| Inhibitor | Catalog No./Brand Name/Sequence/Structure |
|---|---|
| pembrolizumab | KEYTRUDA ® |
| nivolumab | OPDIVO ® |
| cemiplimab | LIBTAYO ® |
| ipilimumab | YERVOY ® |
| tremelimumab | IMJUNO ® |
| durvalumab | IMFINZI ™ |
| dostarlimab | JEMPERLI ® |
| avelumab | BAVENCIO ® |
| atezolizumab | TECENTRIQ ® |
| relatlimab | |
| relatlimab + nivolumab | OPDUALAG ™ |
| BMS-8 | PubChem Compound CID: 117941742 |
| BMS-37 | PubChem Compound CID: 117941658 |
| BMS-202 | PubChem Compound CID: 117951478 |
| BMS-230 | PubChem Compound CID: |
| BMS-242 | PubChem Compound CID: |
| BMS-1001 | PubChem Compound CID: 145925650 |
| BMS-1166 | PubChem Compound CID: 118434635 |
| SB415286 | PubChem Compound CID: 4210951 |
| vorinostat | PubChem Compound CID: 5311 |
| decitabine | PubChem Compound CID: 451668 |
| entitostat | |
| JQ1 | PubChem Compound CID: 46907787 |
| BET151 | PubChem Compound CID: 52912189 |
| GSK503 | PubChem Compound CID: 67469117 |
| panobinostat | PubChem Compound CID: 6918837 |
| ACY-241 | PubChem Compound CID: 53340426 |
| azacytidine | PubChem Compound CID: 9444 |
| DB36 | PubChem Compound CID: 4128273 |
| DB71 | |
| DB15 | PubChem Compound CID: 52278626 |
| CVN | |
| MGCD0103 | PubChem Compound CID: 9865515 |
| SNDX-275 | PubChem Compound CID: 4261 |
| IMP32 | |
| BMS986016 | |
| TSR-022 | |
| Sym023 | |
| ATIK2a | |
| DZNep | PubChem Compound CID: 73087 |

In some cases, an immune checkpoint inhibitor can be as described elsewhere (see, e.g., Smith et al., Am. J. Transl. Res., 11 (2): 529-541 (2019) at, for example, Table 1; Terranova-Barberio et al., Immunotherapy, 8 (6): 705-719 (2016) at, for example, Table 1).

In some cases, one or more immune checkpoint inhibitors can be formulated into a composition (e.g., a pharmaceutically acceptable composition) for administration to a mammal (e.g., a human) having cancer (e.g., a cancer including one or more solid tumors). For example, one or more immune checkpoint inhibitors can be formulated together with one or more pharmaceutically acceptable carriers (additives), excipients, and/or diluents. In some cases, a pharmaceutically acceptable carrier, excipient, or diluent can be a naturally occurring pharmaceutically acceptable carrier, excipient, or diluent. In some cases, a pharmaceutically acceptable carrier, excipient, or diluent can be a non-naturally occurring (e.g., an artificial or synthetic) pharmaceutically acceptable carrier, excipient, or diluent. Examples of pharmaceutically acceptable carriers, excipients, and diluents that can be used in a composition described herein (e.g., a pharmaceutically acceptable composition including one or more immune checkpoint inhibitors) include, without limitation, serum proteins (e.g., human serum albumin), water, and salts or electrolytes (e.g., phosphate salts, saline, protamine sulfate, and DMSO).

In some cases, a population of T cells described herein (e.g., a population of different T cells engineered to each include a CAR) and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered to a mammal at the same time (e.g., in a single composition). In some cases when a population of T cells described herein and an antigenic composition described herein are formulated as a single composition, the T cells can be loaded with the antigenic compositions. For example, T cells in a population of T cells described herein can be contacted with an antigenic composition (e.g., an antigenic composition containing viruses such as oncolytic viruses) such that the antigen(s) (e.g., the viruses) bind to the T cells. In some cases, antigens (e.g., the viruses such as oncolytic viruses) that are loaded onto T cells (e.g., CAR⁺ T cells) can be covalently bound to the surface of the T cells. In some cases, antigens (e.g., the viruses such as oncolytic viruses) that are loaded onto the surface of T cells (e.g., CAR⁺ T cells) can be non-covalently bound to the T cells. In some cases, antigens (e.g., viruses such as oncolytic viruses) that are loaded onto the surface of T cells (e.g., CAR⁺ T cells) can be bound to the T cells through envelope receptor interactions, electrostatic interactions, and/or non-specific interactions between the virus and the T cell surface glycocalyx.

In some cases when a population of T cells described herein (e.g., a population of different T cells engineered to each include a CAR) and an antigenic composition including one or more viruses (e.g., one or more oncolytic viruses) and/or one or more viruses designed to express one or more antigens of interest, are formulated as a single composition, at least some of the T cells can be infected with the virus(es). For example, T cells in a population of T cells described herein can be contacted with antigenic composition including one or more viruses (e.g., one or more oncolytic viruses) and/or one or more viruses designed to express one or more antigens of interest such that the virus(es) can infect at least some of the T cells within the population of T cells.

In some cases when a population of T cells described herein (e.g., a population of different T cells engineered to each include a CAR) and an antigenic composition including one or more viruses (e.g., one or more oncolytic viruses) and/or one or more viruses designed to express one or more antigens of interest, are formulated as a single composition, the population of T cells and the composition containing the viruses can be combined into that single composition in a manner that results in minimal viral infection of the T cells. For example, a population of T cells described herein (e.g., a population of different T cells engineered to each include a CAR) and an antigenic composition including one or more viruses (e.g., one or more oncolytic viruses) and/or one or more viruses designed to express one or more antigens of interest can be combined and incubated at a temperature of about 2° C. to about 8° C. (e.g., about 2° C. to about 6° C., about 2° C. to about 5° C., about 3° C. to about 8° C., about 4° C. to about 8° C., about 3° C. to about 6° C., about 3° C. to about 5° C., or about 4° C.) for 3 hours or less (e.g., 2.5 hours or less, 2 hours or less, 1.5 hours or less, 1 hour or less, or about 1 hour) prior to being administered to the mammal or prior to being frozen for administration to the mammal at a later time. In such cases, the viruses can infect less than about 10 percent (e.g., less than about 9 percent, less than about 8 percent, less than about 7 percent, less than about 7 percent, or less than about 5 percent) of the T cells of the population. For example, when T cells in a population of T cells described herein are loaded with an antigenic composition including one or more viruses, the viruses can infect less than about 5 percent of the T cells of that population.

In some cases, a composition including a population of T cells described herein (e.g., a population of different T cells engineered to each include a CAR) and an antigenic composition described herein (e.g., a composition including one or more viruses such as one or more oncolytic viruses, a composition including one or more viruses designed to express one or more antigens of interest, and/or a composition including one or more antigenic polypeptides of interest) can include an antigenic composition including one or more antigenic polypeptides of interest in the absence of viruses. For example, a composition including a population of T cells described herein and an antigenic composition including one or more antigenic polypeptides of interest can lack the presence of virus particles.

When a population of T cells described herein (e.g., a population of different T cells engineered to each include a CAR) and an antigenic composition described herein (e.g., a composition including one or more viruses such as one or more oncolytic viruses, a composition including one or more viruses designed to express one or more antigens of interest, and/or a composition including one or more antigenic polypeptides of interest) are administered as a single composition, the composition including a population of different T cells engineered to each include a CAR described herein and an antigenic composition described herein can be administered to a mammal by any appropriate route. For example, a composition including (a) a population of T cells described herein and (b) an antigenic composition described herein can be administered locally or systemically. In some cases, a composition including (a) a population of different T cells engineered to each include a CAR described herein and (b) an antigenic composition described herein can be designed for parenteral (e.g., subcutaneous, intramuscular, intravenous, intraperitoneal, and intradermal) administration. Compositions suitable for parenteral administration include aqueous and non-aqueous sterile injection solutions that can contain anti-oxidants, buffers, bacteriostats, and solutes which render the composition isotonic with the blood of the intended recipient; and aqueous and non-aqueous sterile suspensions which may include suspending agents and thickening agents.

In some cases, a composition including (a) a population of T cells described herein (e.g., a population of different T cells engineered to each include a CAR) and (b) an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered systemically by intravenous injection to a mammal (e.g., a human).

In some cases, a population of T cells described herein (e.g., a population of different T cells engineered to each include a CAR) and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered a mammal (e.g., a human) separately. For example, a composition including a population of different T cells engineered to each include a CAR and an antigenic composition can be administered to a mammal at the same time (e.g., concurrently) as independent compositions. When a composition including a population of different T cells engineered to each include a CAR and an antigenic composition are administered concurrently, the composition including a population of different T cells engineered to each include a CAR and the antigenic composition can be administered to a mammal within from about 1 second to about 15 minutes (e.g., about 2 seconds to about 15 minutes, about 5 seconds to about 15 minutes, about 10 seconds to about 15 minutes, about 15 seconds to about 15 minutes, about 1 second to about 10 minutes, about 1 second to about 5 minutes, or about 5 seconds to about 10 minutes) of each other.

In some cases, a composition including a population of T cells described herein (e.g., a population of different T cells engineered to each include a CAR) and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered a mammal (e.g., a human) at different times. When a composition including a population of different T cells engineered to each include a CAR and an antigenic composition are administered at different times, the composition including a population of different T cells engineered to each include a CAR and the antigenic composition can be administered to a mammal with from about 16 minutes to about 48 hours (e.g., about 16 minutes to about 45 hours, about 16 minutes to about 36 hours, about 16 minutes to about 24 hours, about 16 minutes to about 12 hours, about 16 minutes to about 8 hours, about 16 minutes to about 6 hours, about 16 minutes to about 4 hours, about 30 minutes to about 48 hours, about 1 hour to about 48 hours, about 2 hours to about 48 hours, about 4 hours to about 48 hours, about 6 hours to about 48 hours, or 8 hours minutes to about 48 hours) between each administration.

When a composition including a population of T cells described herein (e.g., a population of different T cells engineered to each include a CAR) and an antigenic composition described herein (e.g., a composition including one or more viruses such as one or more oncolytic viruses, a composition including one or more viruses designed to express one or more antigens of interest, and/or a composition including one or more antigenic polypeptides of interest) are administered as separate compositions (e.g., administered concurrently as separate compositions or administered as separate compositions with from about 16 minutes to about 48 hours between each administration), each composition can be administered to a mammal by any appropriate route. In some cases, a composition including a population of T cells described herein and an antigenic composition described herein can be administered by the same route. In some cases, a composition including a population of T cells described herein and an antigenic composition described herein can be administered by different routes.

A composition including a population of T cells described herein (e.g., a population of different T cells engineered to each include a CAR) can be administered to a mammal by any appropriate route. For example, a composition including a population of T cells described herein can be administered locally or systemically. In some cases, a composition including a population of different T cells engineered to each include a CAR described herein can be designed for parenteral (e.g., subcutaneous, intramuscular, intravenous, intraperitoneal, and intradermal) administration. In some cases, a composition including a population of T cells described herein can be administered via an intra-tumoral administration. Compositions suitable for parenteral administration include aqueous and non-aqueous sterile injection solutions that can contain anti-oxidants, buffers, bacteriostats, and solutes which render the composition isotonic with the blood of the intended recipient; and aqueous and non-aqueous sterile suspensions which may include suspending agents and thickening agents.

An antigenic composition described herein (e.g., a composition including one or more viruses such as one or more oncolytic viruses, a composition including one or more viruses designed to express one or more antigens of interest, and/or a composition including one or more antigenic polypeptides of interest) can be administered to a mammal by any appropriate route. For example, an antigenic composition described herein can be administered locally or systemically. In some cases, an antigenic composition described herein can be designed for oral or parenteral (e.g., subcutaneous, intramuscular, intravenous, intraperitoneal, and intradermal) administration. In some cases, an antigenic composition described herein can be administered via an intra-tumoral administration. Compositions suitable for parenteral administration include aqueous and non-aqueous sterile injection solutions that can contain anti-oxidants, buffers, bacteriostats, and solutes which render the composition isotonic with the blood of the intended recipient; and aqueous and non-aqueous sterile suspensions which may include suspending agents and thickening agents. The composition can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

In some cases, a composition including a population of T cells described herein (e.g., a population of different T cells engineered to each include a CAR) can be administered by intravenous injection to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by intravenous injection to the mammal.

In some cases, a composition including a population of T cells described herein (e.g., a population of different T cells engineered to each include a CAR) can be administered by intra-tumoral administration to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by intra-tumoral administration to the mammal.

In some cases, a composition including a population of T cells described herein (e.g., a population of different T cells engineered to each include a CAR) can be administered by intraperitoneal injection to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by intravenous injection to the mammal.

In some cases, a composition including a population of T cells described herein (e.g., a population of different T cells engineered to each include a CAR) can be administered by intravenous injection to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by intraperitoneal injection to the mammal.

In some cases, a composition including a population of T cells described herein (e.g., a population of different T cells engineered to each include a CAR) can be administered by subcutaneous injection to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by intravenous injection to the mammal.

In some cases, a composition including a population of T cells described herein (e.g., a population of different T cells engineered to each include a CAR) can be administered by intravenous injection to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by subcutaneous injection to the mammal.

In some cases, a composition including a population of T cells described herein (e.g., a population of different T cells engineered to each include a CAR) can be administered by intramuscular injection to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by intravenous injection to the mammal.

In some cases, a composition including a population of T cells described herein (e.g., a population of different T cells engineered to each include a CAR) can be administered by intravenous injection to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by intramuscular injection to the mammal.

In some cases, a composition including a population of T cells described herein (e.g., a population of different T cells engineered to each include a CAR) can be administered by intravenous injection to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered orally to the mammal.

When a composition including a population of T cells described herein (e.g., a population of different T cells engineered to each include a CAR) and an antigenic composition described herein (e.g., a composition including one or more viruses such as one or more oncolytic viruses, a composition including one or more viruses designed to express one or more antigens of interest, and/or a composition including one or more antigenic polypeptides of interest) are administered as separate compositions (e.g., administered concurrently as separate compositions or administered as separate compositions with from about 0 seconds to about 15 minutes between each administration), the population of T cells can be administered first, and the antigenic composition administered second, or vice versa.

In some cases, administering (a) a population of T cells described herein (e.g., a population of different T cells engineered to each include a CAR) and (b) an antigenic composition described herein (e.g., a composition including one or more viruses such as one or more oncolytic viruses, a composition including one or more viruses designed to express one or more antigens of interest, and/or a composition including one or more antigenic polypeptides of interest) to mammal (e.g., a human) can be effective to generate memory T cells in vivo. For example, one or more T cells in a population of T cells described herein that are administered to a mammal can be converted into a memory T cell (e.g., a dual-specific memory T cell that is CAR⁺ and that has an endogenous TCR specific for an antigen present within the antigenic composition administered to the mammal). Examples of types of memory T cells that can be generated from T cells (e.g., CAR⁺ T cells) administered to a mammal as described herein include, without limitation, central memory T cells (T_{CM} cells), effector memory T cells (T_{EM} cells), terminally differentiated effector memory T cells (T_{EMRA} cells), and tissue resident memory T cells (T_{RM}). Memory T cells generated within a mammal by the administration of a population of T cells described herein and an antigenic composition described herein to the mammal can be dual-specific. For example, memory T cells generated within a mammal by the administration of CAR⁺ T cells and an antigenic composition to the mammal can target an antigen recognized by the CAR via the CAR and an antigen present in the antigenic composition via an endogenous TCR.

In some cases, a population of T cells described herein (e.g., a population of different T cells engineered to each include a CAR) and one or more immune checkpoint inhibitors can be administered to a mammal at the same time (e.g., in a single composition).

In some cases, a population of T cells described herein (e.g., a population of different T cells engineered to each include a CAR) and one or more immune checkpoint inhibitors can be administered a mammal (e.g., a human) separately. For example, a population of T cells described herein (e.g., a population of different T cells engineered to each include a CAR) and one or more immune checkpoint inhibitors can be administered to a mammal at the same time (e.g., concurrently) as independent compositions. When a composition including a population of T cells described herein (e.g., a population of different T cells engineered to each include a CAR) and one or more immune checkpoint inhibitors are administered concurrently, the composition including a population of T cells and the one or more immune checkpoint inhibitors can be administered to a mammal within from about 1 second to about 15 minutes (e.g., about 2 seconds to about 15 minutes, about 5 seconds to about 15 minutes, about 10 seconds to about 15 minutes, about 15 seconds to about 15 minutes, about 1 second to about 10 minutes, about 1 second to about 5 minutes, or about 5 seconds to about 10 minutes) of each other.

In some cases, a population of T cells described herein (e.g., a population of different T cells engineered to each include a CAR) and one or more immune checkpoint inhibitors can be administered a mammal (e.g., a human) at different times. When a composition including a population of T cells described herein (e.g., a population of different T cells engineered to each include a CAR) and one or more immune checkpoint inhibitors are administered at different times, the composition including a population of T cells and the one or more immune checkpoint inhibitors can be administered to a mammal with from about 16 minutes to about 48 hours (e.g., about 16 minutes to about 45 hours, about 16 minutes to about 36 hours, about 16 minutes to about 24 hours, about 16 minutes to about 12 hours, about 16 minutes to about 8 hours, about 16 minutes to about 6 hours, about 16 minutes to about 4 hours, about 30 minutes to about 48 hours, about 1 hour to about 48 hours, about 2 hours to about 48 hours, about 4 hours to about 48 hours, about 6 hours to about 48 hours, or 8 hours minutes to about 48 hours) between each administration.

When a composition including a population of T cells described herein (e.g., a population of different T cells engineered to each include a CAR) and one or more immune checkpoint inhibitors are administered as separate compositions (e.g., administered concurrently as separate compositions or administered as separate compositions with from about 16 minutes to about 48 hours between each administration), each composition can be administered to a mammal by any appropriate route. In some cases, a composition including a population of T cells described herein (e.g., a population of different T cells engineered to each include a CAR) and one or more immune checkpoint inhibitors can be administered by the same route. In some cases, a population of T cells described herein (e.g., a population of different T cells engineered to each include a CAR) and one or more immune checkpoint inhibitors can be administered by different routes.

One or more immune checkpoint inhibitors described herein (e.g., a composition including one or more immune checkpoint inhibitors) can be administered to a mammal by any appropriate route. For example, one or more immune checkpoint inhibitors described herein can be administered locally or systemically. In some cases, one or more immune checkpoint inhibitors described herein can be designed for oral or parenteral (e.g., subcutaneous, intramuscular, intravenous, intraperitoneal, and intradermal) administration. In some cases, one or more immune checkpoint inhibitors described herein can be administered via an intra-tumoral administration. Compositions suitable for parenteral administration include aqueous and non-aqueous sterile injection solutions that can contain anti-oxidants, buffers, bacteriostats, and solutes which render the composition isotonic with the blood of the intended recipient; and aqueous and non-aqueous sterile suspensions which may include suspending agents and thickening agents. The composition can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

When a composition including a population of T cells described herein (e.g., a population of different T cells engineered to each include a CAR) and one or more immune checkpoint inhibitors are administered as separate compositions (e.g., administered concurrently as separate compositions or administered as separate compositions with from about 0 seconds to about 15 minutes between each administration), the population of T cells can be administered first, and the one or more immune checkpoint inhibitors administered second, or vice versa.

In some cases, memory T cells generated within a mammal (e.g., a human) as described herein (e.g., by administering (a) CAR T cells, (b) one or more antigenic compositions, and (c) one or more immune checkpoint inhibitors) can be more functional against cancer cells present in the mammal (e.g., as compared to T cells such as CAR⁺ T cells that are administered without an antigenic composition and/or without subsequently administering (e.g., boosting) with an antigenic composition). In some cases, memory T cells generated within a mammal (e.g., a human) as described herein (e.g., by administering (a) CAR T cells, (b) one or more antigenic compositions, and (c) one or more immune checkpoint inhibitors) can be more functional against cancer cells present in the mammal (e.g., as compared to T cells such as CAR⁺ T cells that are administered without an antigenic composition and/or without subsequently administering (e.g., boosting) with an antigenic composition) as assessed by, for example, increased cytotoxicity against CAR target cancer cells and/or increased IFN-γ secretion upon stimulation with cancer cells.

Once memory T cells (e.g., T_{RM} CAR⁺ T cells) are generated within a mammal, the mammal can be administered a second antigenic composition (e.g., a composition including one or more viruses such as one or more oncolytic viruses, a composition including one or more viruses designed to express one or more antigens of interest, and/or a composition including one or more antigenic polypeptides of interest) one or more times (e.g., one, two, three, four, five, or more times) and one or more immune checkpoint inhibitors. For example, a mammal can be subsequently administered (e.g., can be boosted with) a second antigenic composition and one or more immune checkpoint inhibitors from about 5 days to about 5 years (e.g., from about 5 days to about 5 years, from about 7 days to about 5 years, from about 10 days to about 5 years, from about 14 days to about 5 years, from about 21 days to about 5 years, from about 1 month to about 5 years, from about 2 months to about 5 years, from about 3 months to about 5 years, from about 4 months to about 5 years, from about 5 months to about 5 years, from about 6 months to about 5 years, from about 5 days to about 4.5 years, from about 5 days to about 4 years, from about 5 days to about 3.5 years, from about 5 days to about 3 years, from about 5 days to about 2.5 years, from about 5 days to about 2 years, from about 5 days to about 1.5 years, from about 5 days to about 1 year, from about 5 days to about 10 months, from about 5 days to about 8 months, from about 5 days to about 6 months, from about 5 days to about 4 months, from about 5 days to about 3 months, from about 5 days to about 2 months, from about 5 days to about 1 month, from about 5 days to about 20 days, from about 5 days to about 15 days, from about 5 days to about 10 days, from about 10 days to about 200 days, from about 20 days to about 200 days, from about 30 days to about 200 days, from about 40 days to about 200 days, from about 50 days to about 200 days, from about 10 days to about 175 days, from about 10 days to about 150 days, from about 10 days to about 125 days, from about 10 days to about 100 days, from about 50 days to about 110 days, or from about 60 days to about 100 days) after having been administered (a) a population of different T cells engineered to each include a CAR and (b) a first antigenic composition (e.g., a composition including one or more viruses such as one or more oncolytic viruses, a composition including one or more viruses designed to express one or more antigens of interest, and/or a composition including one or more antigenic polypeptides of interest). In some cases, a mammal can be administered a second antigenic composition (e.g., a boost) and one or more immune checkpoint inhibitors from about 5 days to about 150 days (e.g., from about 60 days to about 100 days) after having been administered (a) a population of different T cells engineered to each include a CAR and (b) a first antigenic composition. For example, a mammal can be administered a second antigenic composition and one or more immune checkpoint inhibitors from about 5 days to about 8 days (e.g., about 7 days) after having been administered a population of different T cells engineered to each include a CAR and a first antigenic composition.

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) can include the same antigen(s) as a first antigenic composition that was administered together with a population of T cells described herein (e.g., a population of different T cells engineered to each include a CAR).

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) can include one or more different antigens as compared to the first antigenic composition that was administered together with a population of T cells described herein (e.g., a population of different T cells engineered to each include a CAR).

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) can lack T cells (e.g., can lack a population of T cells engineered to each include a CAR). For example, a mammal (e.g., a human) can be administered (a) a population of T cells as described herein (e.g., a population of different T cells engineered to each include a CAR) and (b) a first antigenic composition as described herein (e.g., a composition including one or more viruses such as one or more oncolytic viruses, a composition including one or more viruses designed to express one or more antigens of interest, and/or a composition including one or more antigenic polypeptides of interest). Then, after at least about 5 days (e.g., after at least about 7 days, after at least about 10 days, after at least about 14 days, after at least about 20 days, after at least about 50 days, after at least about 60 days, after at least about 75 days, after at least about 3 months, after at least about 4 months, after at least about 5 months, or after at least about 6 months) after the latter administration of that population of T cells and that first antigenic composition, the mammal (e.g., the human) can be administered a second antigenic composition that does not include T cells. In some cases, that second antigenic composition can be identical to the first antigenic composition administered to the mammal. For example, in some cases, the first antigenic composition administered to the mammal can include one or more oncolytic viruses (e.g., one or more VSV viruses, one or more reoviruses, one or more measles viruses, or combinations thereof), and the second antigenic composition administered to the mammal can include those same one or more oncolytic viruses. In some cases, that second antigenic composition can be different from the first antigenic composition administered to the mammal. For example, in some cases, the first antigenic composition administered to the mammal can include one or more viruses designed to express one or more antigens of interest, and the second antigenic composition administered to the mammal can include one or more of those antigens of interest that were expressed by the viruses of the first antigenic composition with that second antigenic composition lacking the viruses. In some cases, a mammal (e.g., a human) can be treated as described herein with the initially administered population of T cells (e.g., a population of different T cells engineered to each include a CAR) being the only T cells that are administered to the mammal.

In some cases, a mammal (e.g., a human) can be treated as described herein with the initially administered population of T cells (e.g., a population of different T cells engineered to each include a CAR) and first antigenic composition can be subsequently treated with multiple rounds of additional populations of T cells (e.g., an additional population of different T cells engineered to each include a CAR) and/or additional antigenic compositions.

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) can include one or more viruses (e.g., one or more oncolytic viruses).

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) can include one or more antigenic polypeptides of interest. For example, a second antigenic composition can include one or more antigenic polypeptides of interest that were expressed by a virus present in a first antigen composition. For example, a second antigenic composition can include one or more antigenic polypeptides of interest that were expressed by a virus present in a first antigen composition, and can lack virus particles.

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) can be administered to a mammal (e.g., a human) as the sole active agent to stimulate the memory T cells generated within the mammal.

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) can be administered to a mammal (e.g., a human) together with one or more additional agents that can stimulate memory T cells within the mammal (e.g., can stimulate the memory T cells generated within the mammal as described herein). Examples of additional agents (e.g., other than a second antigen composition) that can be used to stimulate memory T cells within a mammal include, without limitation, pathogens and TLR agonists.

In some cases, administration of a second antigenic composition (e.g., a composition including one or more viruses such as one or more oncolytic viruses, a composition including one or more viruses designed to express one or more antigens of interest, and/or a composition including one or more antigenic polypeptides of interest) as described herein (e.g., a boost) can be effective to activate memory T cells (e.g., dual-specific memory T cells) generated as described herein. For example, a subsequent administration (e.g., a boost) of a second antigenic composition can be used to rapidly reactivate memory T cells generated by administering a population of T cells described herein and a first antigenic composition described herein to generate effector T cells that are dual-specific (e.g., effector T cells that are CAR⁺ and positive for an endogenous TCR that recognizes an antigen that was present in both the first antigenic composition and the boost).

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) and one or more immune checkpoint inhibitors can be administered to a mammal at the same time (e.g., in a single composition).

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) and one or more immune checkpoint inhibitors can be administered a mammal (e.g., a human) separately. For example, a second antigenic composition (e.g., a composition including one or more viruses such as one or more oncolytic viruses, a composition including one or more viruses designed to express one or more antigens of interest, and/or a composition including one or more antigenic polypeptides of interest) and one or more immune checkpoint inhibitors can be administered to a mammal at the same time (e.g., concurrently) as independent compositions. When a composition including a second antigenic composition (e.g., a composition including one or more viruses such as one or more oncolytic viruses, a composition including one or more viruses designed to express one or more antigens of interest, and/or a composition including one or more antigenic polypeptides of interest) and one or more immune checkpoint inhibitors are administered concurrently, the composition including a second antigenic composition and the one or more immune checkpoint inhibitors can be administered to a mammal within from about 1 second to about 15 minutes (e.g., about 2 seconds to about 15 minutes, about 5 seconds to about 15 minutes, about 10 seconds to about 15 minutes, about 15 seconds to about 15 minutes, about 1 second to about 10 minutes, about 1 second to about 5 minutes, or about 5 seconds to about 10 minutes) of each other.

In some cases, a composition including a second antigenic composition (e.g., a composition including one or more viruses such as one or more oncolytic viruses, a composition including one or more viruses designed to express one or more antigens of interest, and/or a composition including one or more antigenic polypeptides of interest) and one or more immune checkpoint inhibitors can be administered a mammal (e.g., a human) at different times. When a composition including a second antigenic composition (e.g., a composition including one or more viruses such as one or more oncolytic viruses, a composition including one or more viruses designed to express one or more antigens of interest, and/or a composition including one or more antigenic polypeptides of interest) and one or more immune checkpoint inhibitors are administered at different times, the composition including a second antigenic composition and the one or more immune checkpoint inhibitors can be administered to a mammal with from about 16 minutes to about 48 hours (e.g., about 16 minutes to about 45 hours, about 16 minutes to about 36 hours, about 16 minutes to about 24 hours, about 16 minutes to about 12 hours, about 16 minutes to about 8 hours, about 16 minutes to about 6 hours, about 16 minutes to about 4 hours, about 30 minutes to about 48 hours, about 1 hour to about 48 hours, about 2 hours to about 48 hours, about 4 hours to about 48 hours, about 6 hours to about 48 hours, or 8 hours minutes to about 48 hours) between each administration.

When a composition including a second antigenic composition (e.g., a composition including one or more viruses such as one or more oncolytic viruses, a composition including one or more viruses designed to express one or more antigens of interest, and/or a composition including one or more antigenic polypeptides of interest) and one or more immune checkpoint inhibitors are administered as separate compositions (e.g., administered concurrently as separate compositions or administered as separate compositions with from about 16 minutes to about 48 hours between each administration), each composition can be administered to a mammal by any appropriate route. In some cases, a composition including a second antigenic composition (e.g., a composition including one or more viruses such as one or more oncolytic viruses, a composition including one or more viruses designed to express one or more antigens of interest, and/or a composition including one or more antigenic polypeptides of interest) and one or more immune checkpoint inhibitors can be administered by the same route. In some cases, a second antigenic composition (e.g., a composition including one or more viruses such as one or more oncolytic viruses, a composition including one or more viruses designed to express one or more antigens of interest, and/or a composition including one or more antigenic polypeptides of interest) and one or more immune checkpoint inhibitors can be administered by different routes.

A second antigenic composition (e.g., a composition including one or more viruses such as one or more oncolytic viruses, a composition including one or more viruses designed to express one or more antigens of interest, and/or a composition including one or more antigenic polypeptides of interest) can be administered to a mammal by any appropriate route. For example, a second antigenic composition described herein can be administered locally or systemically. In some cases, a second antigenic composition described herein can be designed for oral or parenteral (e.g., subcutaneous, intramuscular, intravenous, intraperitoneal, and intradermal) administration. When being administered orally, a composition can be in the form of a pill, tablet, or capsule. Compositions suitable for parenteral administration include aqueous and non-aqueous sterile injection solutions that can contain anti-oxidants, buffers, bacteriostats, and solutes which render the composition isotonic with the blood of the intended recipient; and aqueous and non-aqueous sterile suspensions which may include suspending agents and thickening agents. The composition can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

In some cases, a second antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by intravenous injection to the mammal.

One or more immune checkpoint inhibitors described herein (e.g., a composition including one or more immune checkpoint inhibitors) can be administered to a mammal by any appropriate route. For example, one or more immune checkpoint inhibitors described herein can be administered locally or systemically. In some cases, one or more immune checkpoint inhibitors described herein can be designed for oral or parenteral (e.g., subcutaneous, intramuscular, intravenous, intraperitoneal, and intradermal) administration. In some cases, one or more immune checkpoint inhibitors described herein can be administered via an intra-tumoral administration. Compositions suitable for parenteral administration include aqueous and non-aqueous sterile injection solutions that can contain anti-oxidants, buffers, bacteriostats, and solutes which render the composition isotonic with the blood of the intended recipient; and aqueous and non-aqueous sterile suspensions which may include suspending agents and thickening agents. The composition can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

When a composition including a second antigenic composition (e.g., a composition including one or more viruses such as one or more oncolytic viruses, a composition including one or more viruses designed to express one or more antigens of interest, and/or a composition including one or more antigenic polypeptides of interest) and one or more immune checkpoint inhibitors are administered as separate compositions (e.g., administered concurrently as separate compositions or administered as separate compositions with from about 0 seconds to about 15 minutes between each administration), the population of T cells can be administered first, and the antigenic composition administered second, or vice versa.

One or more immune checkpoint inhibitors can be administered to a mammal (e.g., a human) having cancer (e.g., a cancer including one or more solid tumors) in any appropriate amount (e.g., any appropriate dose). In some cases, an effective dose of one or more immune checkpoint inhibitors can be a flat dose. In some cases, as effective dose of one or more immune checkpoint inhibitors can be based on the body of a mammal (e.g., a human) to be treated as described herein. An effective amount of one or more immune checkpoint inhibitors can be any amount that can treat a mammal having cancer without producing significant toxicity to the mammal.

The effective amount of one or more immune checkpoint inhibitors can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the cancer (e.g., a cancer including one or more solid tumors) in the mammal being treated may require an increase or decrease in the actual effective amount administered.

One or more immune checkpoint inhibitors can be administered to a mammal (e.g., a human) having cancer (e.g., a cancer including one or more solid tumors) at any appropriate frequency. The frequency of administration can be any frequency that can treat a mammal having cancer without producing significant toxicity to the mammal. For example, the frequency of administration can be from about twice a day to about one every other day, from about once a day to about once a week, from about once a day to about once a month, from about once a week to about once a month, or from about twice a month to about once a month. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

One or more immune checkpoint inhibitors can be administered to a mammal (e.g., a human) having cancer (e.g., a cancer including one or more solid tumors) for any appropriate duration. An effective duration can be any duration that can treat a mammal having cancer without producing significant toxicity to the mammal. For example, the effective duration can vary from several weeks to several months, from several months to several years, or from several years to a lifetime. Multiple factors can influence the actual effective duration used for a particular treatment. For example, an effective duration can vary with the frequency of administration, effective amount, use of multiple treatment agents, and/or route of administration.

In some cases, a mammal (e.g., a human) having cancer (e.g., a cancer including one or more solid tumors) can be administered (a) a population of different T cells that each express a particular CAR in addition to their endogenous TCR, (b) an antigenic composition, and (c) nivolumab. For example, a mammal having cancer can be administered (a) a population of different T cells that each express a particular CAR in addition to their endogenous TCR, (b) an antigenic composition, and (c) 240 mg of nivolumab once every 2 weeks (Q2W). For example, a mammal having cancer can be administered (a) a population of different T cells that each express a particular CAR in addition to their endogenous TCR, (b) an antigenic composition, and (c) 480 mg of nivolumab once every 4 weeks (Q4W). For example, a mammal having cancer can be administered (a) a population of different T cells that each express a particular CAR in addition to their endogenous TCR, (b) an antigenic composition, and (c) 3 mg/kg of nivolumab Q2W.

In some cases, a mammal (e.g., a human) having cancer (e.g., a cancer including one or more solid tumors) can be administered (a) a population of different T cells that each express a particular CAR in addition to their endogenous TCR, (b) an antigenic composition, and (c) pembrolizumab. For example, a mammal having cancer can be administered (a) a population of different T cells that each express a particular CAR in addition to their endogenous TCR, (b) an antigenic composition, and (c) 200 mg of pembrolizumab once every 3 weeks (Q3W). For example, a mammal having cancer can be administered (a) a population of different T cells that each express a particular CAR in addition to their endogenous TCR, (b) an antigenic composition, and (c) 400 mg of pembrolizumab once every 6 weeks (Q6W). For example, a mammal having cancer can be administered (a) a population of different T cells that each express a particular CAR in addition to their endogenous TCR, (b) an antigenic composition, and (c) 2 mg/kg of pembrolizumab Q3W.

In some cases, a mammal (e.g., a human) having cancer (e.g., a cancer including one or more solid tumors) can be administered (a) a population of different T cells that each express a particular CAR in addition to their endogenous TCR, (b) an antigenic composition, and (c) avelumab. For example, a mammal having cancer can be administered (a) a population of different T cells that each express a particular CAR in addition to their endogenous TCR, (b) an antigenic composition, and (c) 800 mg of avelumab Q2W.

In some cases, a mammal (e.g., a human) having cancer (e.g., a cancer including one or more solid tumors) can be administered (a) a population of different T cells that each express a particular CAR in addition to their endogenous TCR, (b) an antigenic composition, and (c) cemiplimab. For example, a mammal having cancer can be administered (a) a population of different T cells that each express a particular CAR in addition to their endogenous TCR, (b) an antigenic composition, and (c) 350 mg of cemiplimab Q3W.

In some cases, one or more immune checkpoint inhibitors can be administered using a dose and administration regimen as described elsewhere (see, e.g., Jiang et al., Front Oncol., 12:906251 (2002) at, for example, Table 1; Maritaz et al., J. Hematol. Oncol., 15:6 (2022) at, for example, Table 1).

In some cases, methods for treating a mammal (e.g., a human) as described herein (e.g., by administering (a) CAR T cells, (b) one or more antigenic compositions, and (c) one or more immune checkpoint inhibitors) also can include administering to the mammal one or more (e.g., one, two, three, or more) additional agents used to treat cancer and/or performing one or more (e.g., one, two, three, or more) therapies used to treat cancer. For example, a combination therapy used to treat a mammal (e.g., a human) having cancer (e.g., a cancer including one or more solid tumors) can include administering to the mammal (a) CAR T cells, (b) one or more antigenic compositions, and (c) one or more immune checkpoint inhibitors, and administering to the mammal one or more (e.g., one, two, three, or more) additional agents used to treat cancer. In some cases, an additional agent that can be administered to a mammal to treat cancer can be a chemotherapeutic agent. In some cases, an additional agent that can be administered to a mammal to treat cancer can be a cytotoxic agent. In some cases, an additional agent that can be administered to a mammal to treat cancer can be an angiogenesis inhibitor. Examples of additional agents that can be administered to a mammal (e.g., a human) having cancer (e.g., a cancer including one or more solid tumors) to treat the mammal include, without limitation, sorafenib, regorafenib, ramucirumab, axitinib (e.g., INLYTA®), bevacizumab (e.g., AVASTIN®), cabozantinib (e.g., COMETRIQ®), and any combinations thereof. In cases where (a) CAR T cells, (b) one or more antigenic compositions, and (c) one or more immune checkpoint inhibitors are used in combination with additional agents used to treat a mammal (e.g., a human) having cancer (e.g., a cancer including one or more solid tumors), the one or more additional agents can be administered at the same time (e.g., in a single composition containing (a) CAR T cells, (b) one or more antigenic compositions, and (c) one or more immune checkpoint inhibitors, and containing the one or more additional agents) or independently. For example, a composition including (a) CAR T cells, (b) one or more antigenic compositions, and (c) one or more immune checkpoint inhibitors can be administered first, and the one or more additional agents administered second, or vice versa.

In some cases, a combination therapy used to treat a mammal (e.g., a human) having cancer (e.g., a cancer including one or more solid tumors) can include administering to the mammal (a) CAR T cells, (b) one or more antigenic compositions, and (c) one or more immune checkpoint inhibitors, and can include performing one or more (e.g., one, two, three, or more) therapies used to treat cancer. Examples of additional therapies that can be used to treat a mammal (e.g., a human) having cancer (e.g., a cancer including one or more solid tumors) include, without limitation, radiation therapies, and/or surgeries. In cases where (a) CAR T cells, (b) one or more antigenic compositions, and (c) one or more immune checkpoint inhibitors are used in combination with one or more therapies used to treat a mammal (e.g., a human) having cancer (e.g., a cancer including one or more solid tumors), the one or more additional therapies can be performed at the same time or independently of the administration of the (a) CAR T cells, (b) one or more antigenic compositions, and (c) one or more immune checkpoint inhibitors. For example, (a) CAR T cells, (b) one or more antigenic compositions, and (c) one or more immune checkpoint inhibitors can be administered before, during, or after the one or more additional therapies are performed.

In some cases, the materials and methods provided herein can be used to improve survival of a mammal (e.g., a human) having cancer. For example, a mammal in need thereof (e.g., a mammal having cancer such as a cancer including one or more solid tumors) can be administered (a) a population of T cells described herein (e.g., a population of different T cells engineered to each include a CAR) and (b) an antigenic composition described herein (e.g., a composition including one or more viruses such as one or more oncolytic viruses, a composition including one or more viruses designed to express one or more antigens of interest, and/or a composition including one or more antigenic polypeptides of interest), and after at least about 5 days (e.g., after at least about 7 days, after at least about 10 days, after at least about 14 days, after at least about 20 days, after at least about 50 days, after at least about 60 days, after at least about 75 days, after at least about 3 months, after at least about 4 months, after at least about 5 months, or after at least about 6 months), can be subsequently administered (a) a second antigenic composition (e.g., a composition including one or more viruses such as one or more oncolytic viruses, a composition including one or more viruses designed to express one or more antigens of interest, and/or a composition including one or more antigenic polypeptides of interest), and (b) one or more immune checkpoint inhibitors to improve survival of the mammal. For example, the materials and methods described herein can be used to improve the survival of a mammal having cancer by, for example, 10, 20, 30, 40, 50, 60, 70, 80, 90, 95, or more percent. For example, the materials and methods described herein can be used to improve the survival of a mammal having cancer by, for example, at least 6 months (e.g., about 6 months, about 8 months, about 10 months, about 1 year, about 1.5 years, about 2 years, about 2.5 years, about 3 years, about 4 years, about 5 years, or more).

In some cases, the materials and methods provided herein can be used to reduce the size of the cancer in the mammal. For example, a mammal in need thereof (e.g., a mammal having cancer such as a cancer including one or more solid tumors) can be administered (a) a population of T cells described herein (e.g., a population of different T cells engineered to each include a CAR) and (b) an antigenic composition described herein (e.g., a composition including one or more viruses such as one or more oncolytic viruses, a composition including one or more viruses designed to express one or more antigens of interest, and/or a composition including one or more antigenic polypeptides of interest), and after at least about 5 days (e.g., after at least about 7 days, after at least about 10 days, after at least about 14 days, after at least about 20 days, after at least about 50 days, after at least about 60 days, after at least about 75 days, after at least about 3 months, after at least about 4 months, after at least about 5 months, or after at least about 6 months), can be subsequently administered a (a) second antigenic composition (e.g., a composition including one or more viruses such as one or more oncolytic viruses, a composition including one or more viruses designed to express one or more antigens of interest, and/or a composition including one or more antigenic polypeptides of interest) and (b) one or more immune checkpoint inhibitors to reduce the size of the cancer in the mammal. In some cases, the methods and materials provided herein can be used as described herein to reduce the number of cancer cells in the mammal by, for example, 10, 20, 30, 40, 50, 60, 70, 80, 90, 95, or more percent. In some cases, the methods and materials provided herein can be used as described herein to reduce the volume of one or more tumors in the mammal by, for example, 10, 20, 30, 40, 50, 60, 70, 80, 90, 95, or more percent.

In some cases, the materials and methods provided herein can include monitoring the mammal (e.g., the human) being treated as described herein (e.g., by administering (a) CAR T cells, (b) one or more antigenic compositions, and (c) one or more immune checkpoint inhibitors). For example, the size of the cancer (e.g., the number of cancer cells and/or the volume of one or more tumors) present within a mammal can be monitored. Any appropriate method can be used to determine whether or not the size of the cancer present within a mammal is reduced. For example, imaging techniques can be used to assess the size of the cancer present within a mammal (e.g., a human).

The invention will be further described in the following examples, which do not limit the scope of the invention described in the claims.

### EXAMPLES

### Example 1: Oncolytic Virus-Mediated Expansion of Dual-Specific CAR T Cells Improves Efficacy Against Solid Tumors in Mice

This Example describes using CAR⁺ T cells and viruses to treat cancer. CAR⁺ T cells and viruses were combined ex vivo and systemically delivered to mice having tumors to generate dual-specific, T_{RM} CAR⁺ T cells that can target (e.g., target and destroy) tumor cells and cause tumor regression.

### Results

Combination of CAR T Cells with Oncolytic VSV Primes Virus-Specific CAR T Cells In Vivo

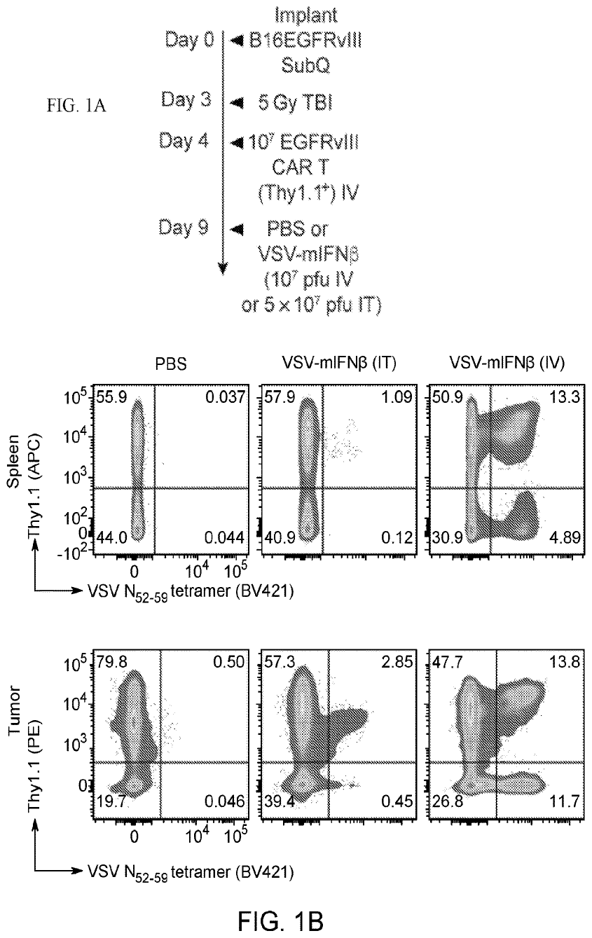

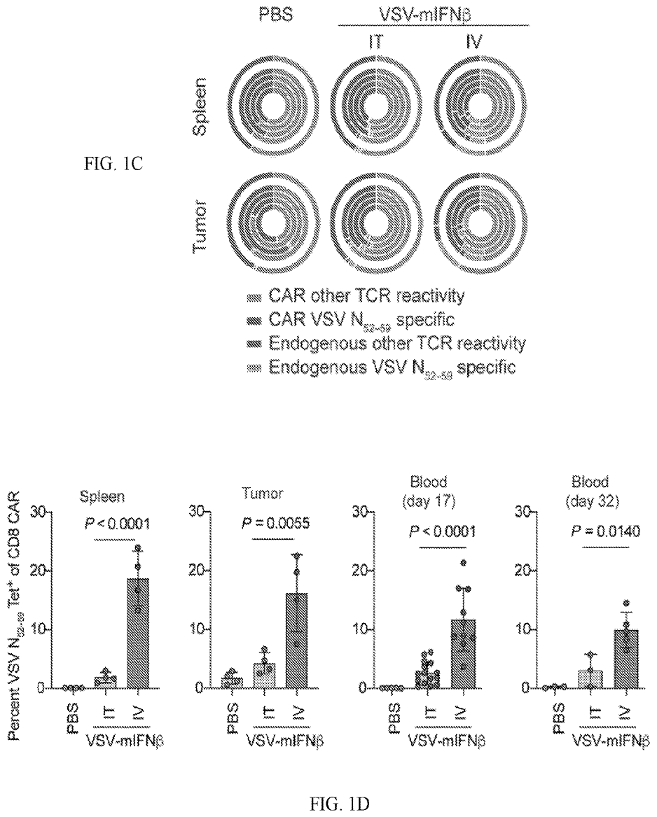

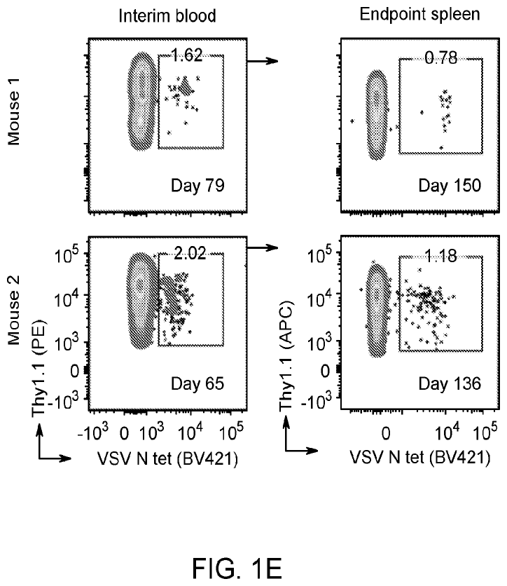

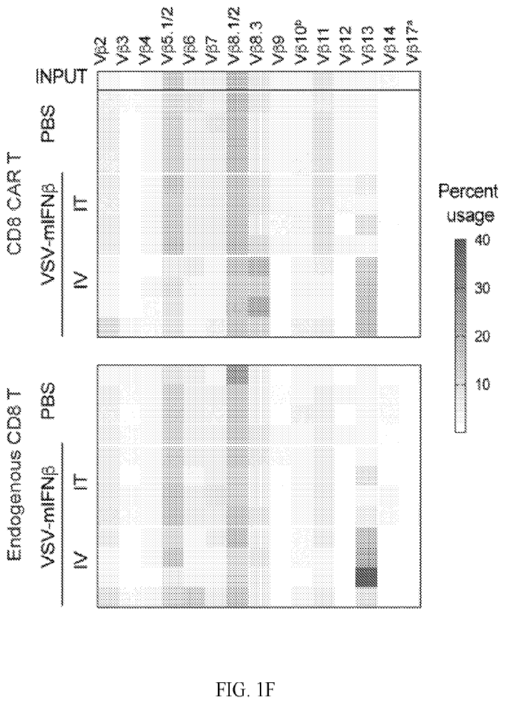

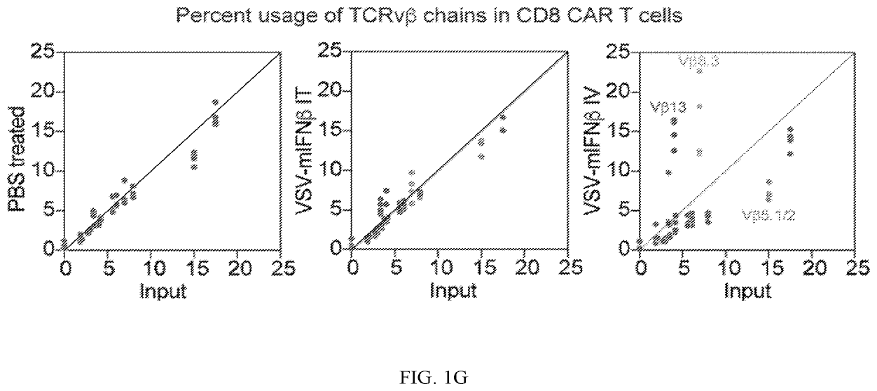

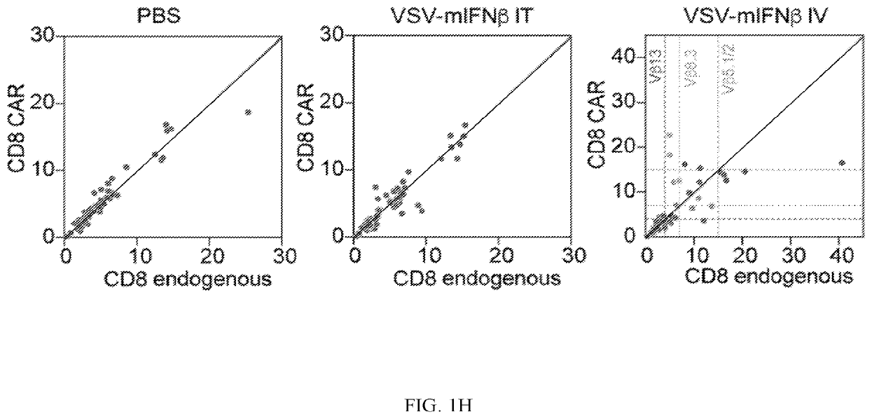

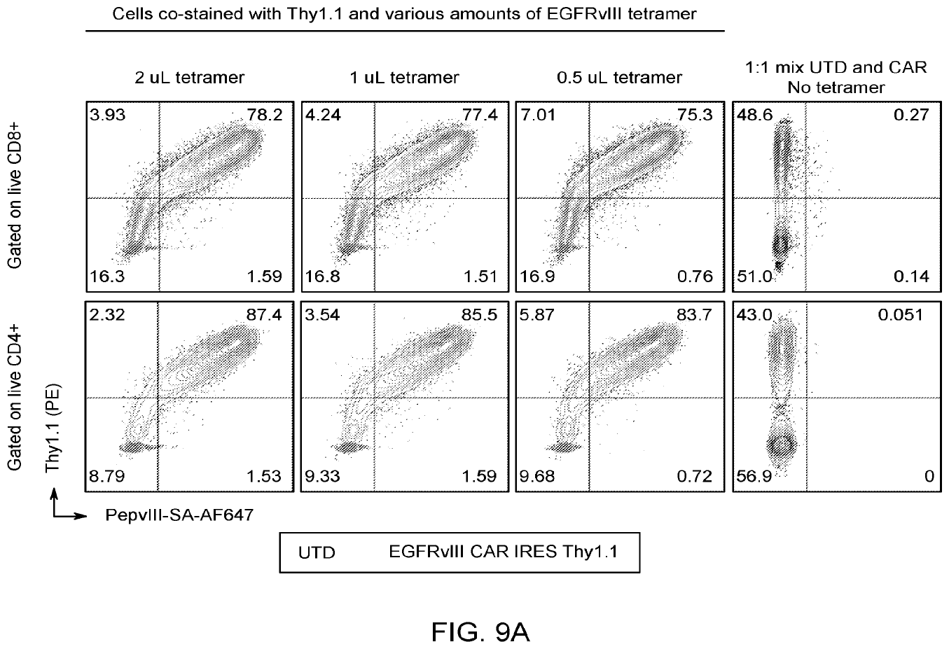

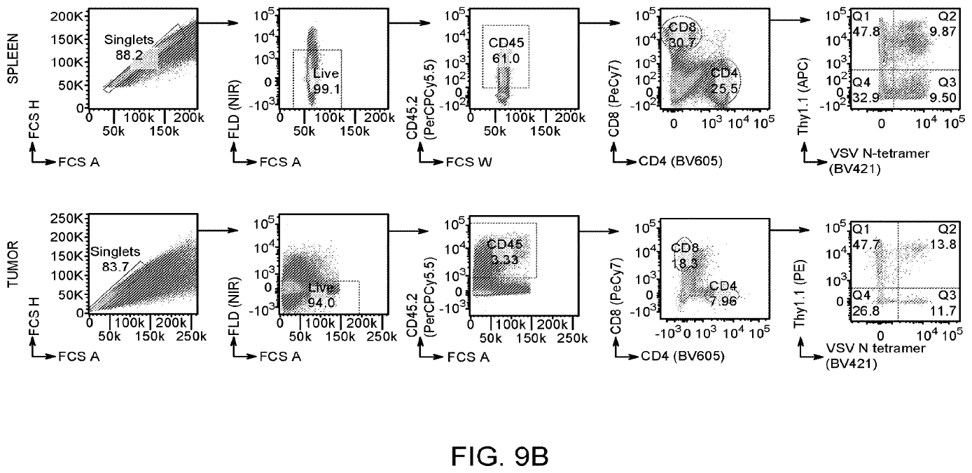

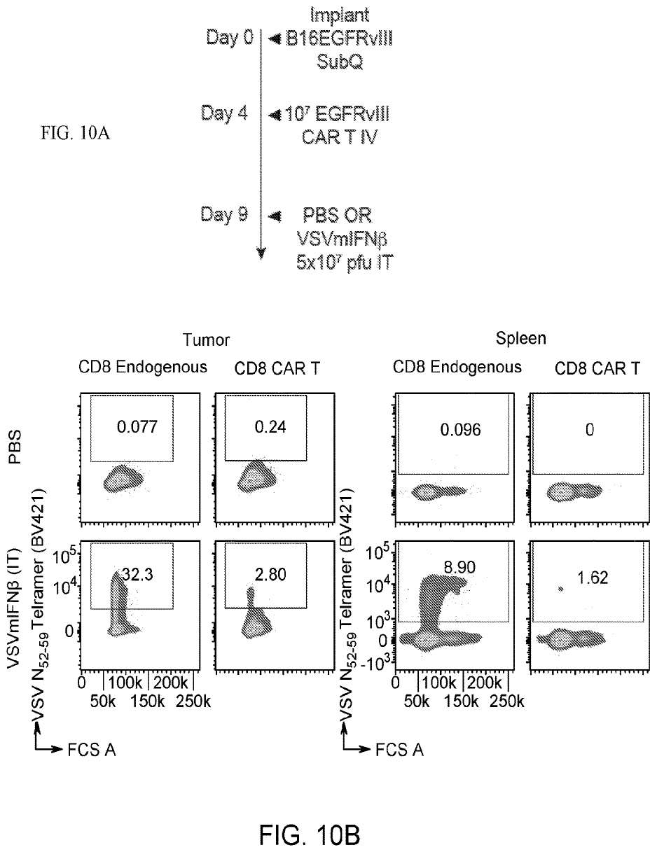

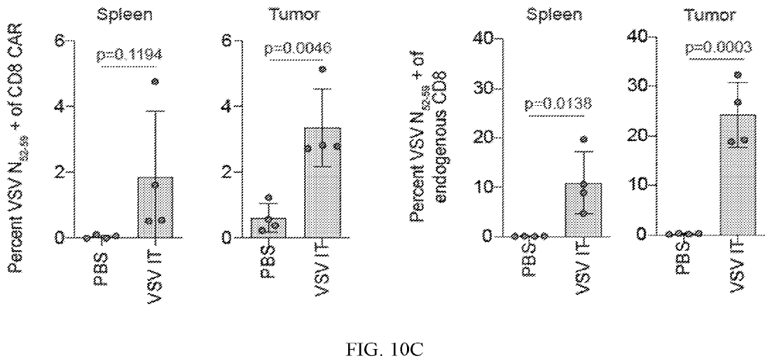

In previous studies, it was tested whether the combination of third-generation anti-epidermal growth factor receptor variant III (EGFRvIII) CAR T cell with intratumoral oncolytic VSV expressing mouse interferon-β (mIFNβ) would provide synergistic interactions in a subcutaneous B16EGFRvIII mouse melanoma tumor model. However, tumor preconditioning with VSV-mIFNβ resulted in virus-induced type I IFN-mediated attrition of activated CAR T cells as they were recruited to the tumor site (Evgin et al., Nat. Commun. 11:3187 (2020)). Using this same model, an alternative schedule was explored where administration of CAR T cells was followed 5 days later by either an intratumoral or intravenous dose of VSV-mIFNβ. CAR T cell attrition was not observed in the tumor, spleen, or blood of virus-treated mice (FIG. 8). CAR T cells were identified using the Thy1.1 marker expressed from the retroviral vector (FIG. 9A). A selective expansion of CD8 CAR T cells was also observed with native TCR specificity to the immunodominant epitope VSV N₅₂₋₅₉ (FIGS. 1A to 1C). VSV N₅₂₋₅₉-specific CAR T cells were detected after both intratumoral and intravenous virus administration, although the expansion was greater in animals that received intravenous virus (FIG. 1D). An exemplary gating scheme is shown in FIG. 9B. The frequency of DS CAR T cells was greatest 7 days after virus exposure and, although it contracted over time, was still detectable in the blood and spleen over 100 days after transfer (FIG. 1E). The expansion of DS CD8 CAR T cells was enhanced by lymphodepletion. In nonpreconditioned mice, although a proportionally similar expansion of virus-specific CD8 CAR T was observed, numerically, this was very low because of limited CAR T engraftment. The endogenous VSV N-specific CD8 population was larger than the VSV N-specific CD8 CAR T cell population (FIG. 10).

The CAR T cells generated in FIG. 1 were derived from an open repertoire of unselected T cells from naïve mice, in which the reported precursor frequency of T cells with TCR specificity for H-2K^{b}-restricted VSV N₅₂₋₅₉ was ~8.24×10⁻⁴% of CD8 T cells. Assuming that the CAR T cell product was ~70% CD8 cells, from a dose of 10⁷ cells, approximately 50 T cells had specificity for the VSV N₅₂₋₅₉ epitope. Despite this low predicted frequency in the input product, a subsequent expansion of this DS population to ~10 to 20% of the CD8 CAR T cell population in vivo was observed (FIG. 1).

Neither splenocytes from naïve mice nor CAR T cells generated from naïve mice specifically produced IFNγ upon restimulation with the minimal MHC-restricted VSV N₅₂₋₅₉ viral epitope, an irrelevant foreign ovalbumin (OVA) antigen (SIINFEKL; SEQ ID NO:1), or the melanoma tumor-associated antigen GP100, thus confirming that these donor mice had not been previously exposed to these antigens (FIGS. 11A and 11B). In addition, the expansion of CD8 CAR T with TCR reactivity against VSV N₅₂₋₅₉ antigen was not dependent on the presence or absence of the viral IFNβ transgene as equal expansion of VSV N₅₂₋₅₉ TCR specific CAR T were detected after stimulation with VSV-mIFNβ or VSV-hGP100 or VSV-OVA (FIG. 11C).

To evaluate how virus administration alters the clonality of the CAR T cell population, a panel of antibodies recognizing commonly used TCR variable beta (VB) chains was used to profile the injected CAR T cells, as well as those cells recovered from mice treated with phosphate-buffered saline (PBS) or VSV-mIFNβ (intratumorally or intravenously). The repertoire of VB chain usage of the CAR T cell population from animals that received PBS was very similar to the injected CAR T cells (FIGS. 1F and 1G). After intravenous VSV-mIFNβ, the profile of TCR VB usage was shifted from that of the input population, with a high degree of selection for CAR T cells using the Vβ13 and Vb8.3 chains and a reduced utilization of Vβ5.1/2 (FIGS. 1F and 1G). Consistent with the tetramer staining showing that intratumoral administration of virus induced a more modest expansion of DS CAR T, minor changes were observed in the VB chain usage in mice that received intratumoral virus. In animals treated with intravenous VSV-mIFNβ, a similar selection of usage of the Vβ13 TCR was observed in the endogenous CD8 T cell population (FIG. 1H).

### DS CAR T Cells Acquire Distinct Functional and Phenotypic Properties

The fact that the virus-specific CAR T cells could reach up to 25% of the CD8 CAR T cell population in vivo indicated that there was a selective advantage for the expansion and survival of these cells over those with no adjunct TCR signaling or activation. In addition to this proliferative advantage, sorted CD8 CAR T cells isolated from mice treated with CAR T cells and VSV-mIFNβ exhibited robust killing of B16EGFRvIII cells over B16 parental cells in vitro (FIGS. 2A and 2B). In contrast, at a low effector:target:nontarget (E:T:NT) ratio of 2:1:1, CAR T cells recovered from mice treated with PBS did not specifically kill EGFRvIII-expressing tumor cells.

Both VSV N-specific and -nonspecific CD8 CAR T cells from virus-treated mice would have been exposed to the same inflammatory environment, and therefore, to more specifically interrogate whether the DS CAR T cells were more active, we examined degranulation and cytokine expression in VSV N₅₂₋₅₉ tetramer-stained samples. As expected, when restimulated with the VSV N₅₂₋₅₉ peptide, CD107a expression, IFNγ, and tumor necrosis factor-α (TNFα) production were specifically observed in the VSV N₅₂₋₅₉ tetramer-stained population (FIGS. 2C to 2E). When sorted CD8 CAR T cells were cocultured with B16EGFRvIII cells, the highest frequency of degranulation and cytokine production was observed from the tetramer-positive population. However, the tetramer-negative population from virus-treated animals also underwent enhanced degranulation and expressed higher concentrations of IFNγ than the CD8 CAR T cells from PBS-treated animals.

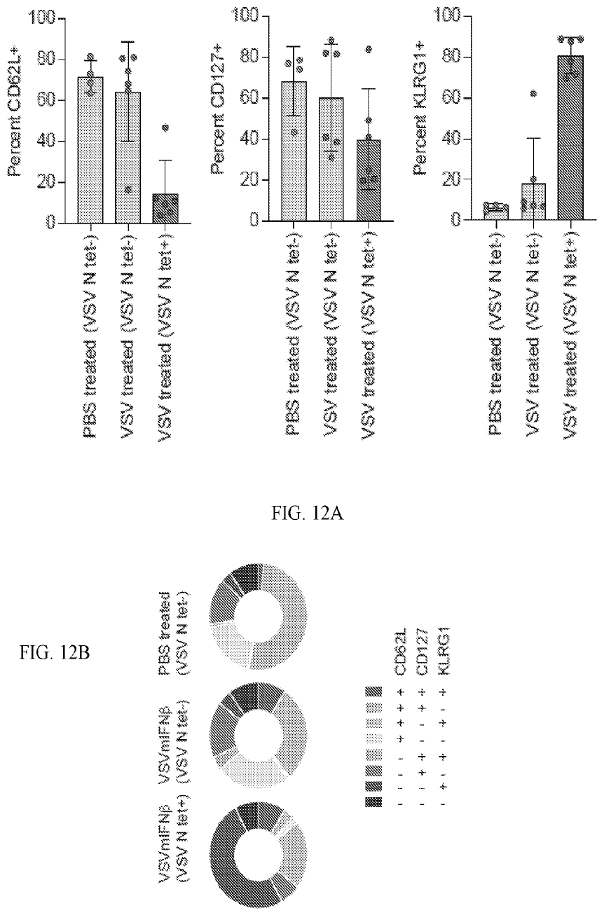

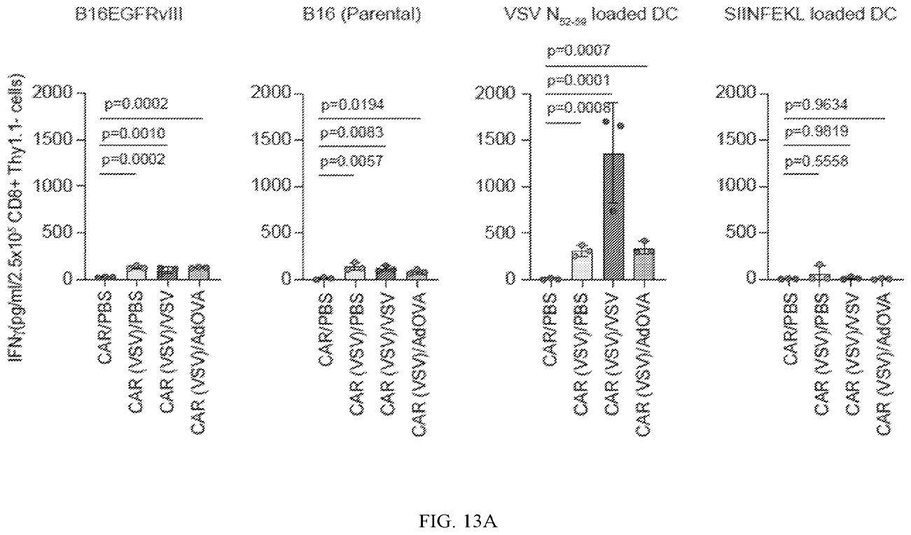

Consistent with these functional differences, VSV N₅₂₋₅₉-specific CD8 CAR T cells had a predominantly KLRG1^{hi}, CD127^{lo}, and CD62L^{lo} effector memory phenotype that was different to that of VSV N-nonspecific CD8 CAR T cells from virus-treated mice and CD8 CAR T cells from PBS-treated mice (FIGS. 2F and 2G). Nonetheless, the KLRG1, CD62L, and CD127 profiles among the CD8 CAR T cells from PBS- and virus-treated animals were very similar to the corresponding endogenous CD8 T cell populations (FIG. 12).

Together, these data suggest that the strong signaling through the TCR resulting from MHC presentation of viral antigens to an open TCR repertoire of CAR T cells promoted a powerful selective survival and proliferative advantage. Moreover, this signaling was associated with improved function against the CAR antigen and an altered memory differentiation profile.

Systemic Tumor Treatment with Virus-Loaded CAR T Cells Expands DS CAR Tin Nonpreconditioned Animals

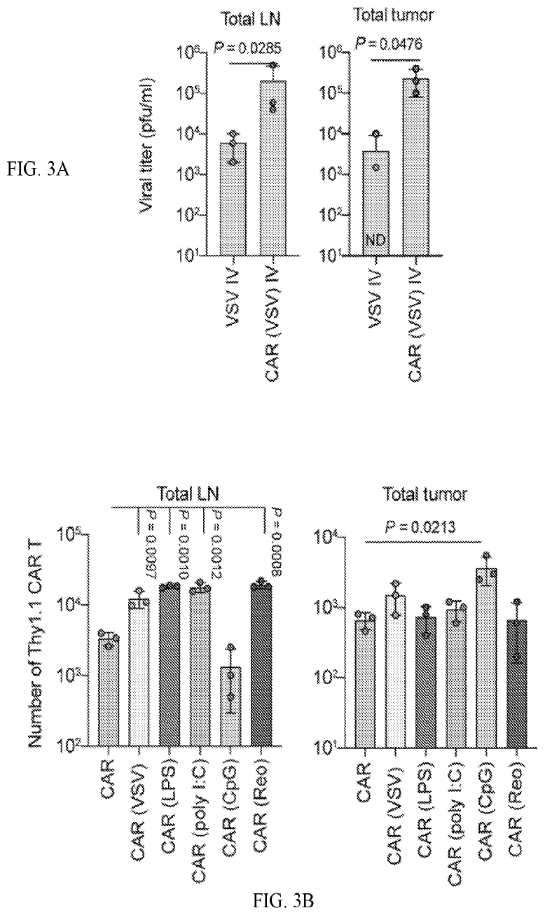

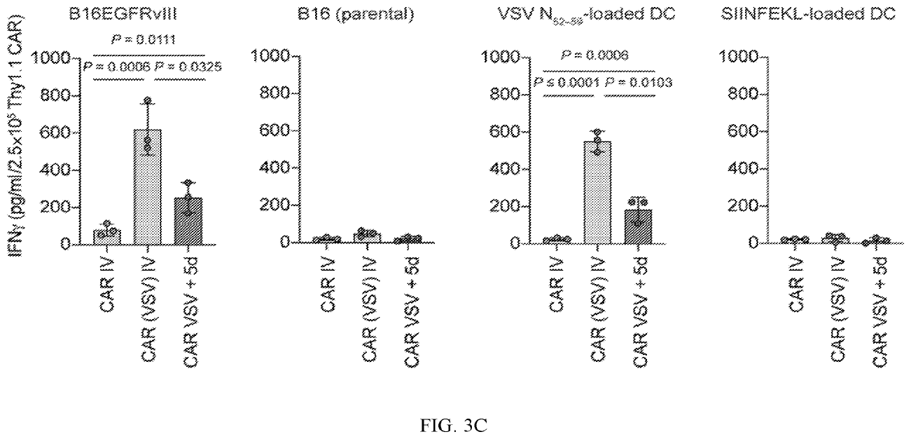

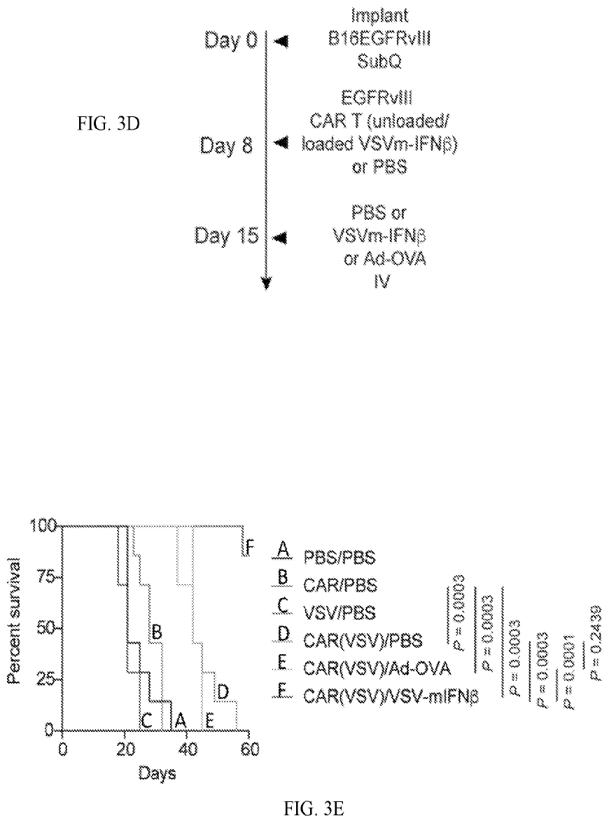

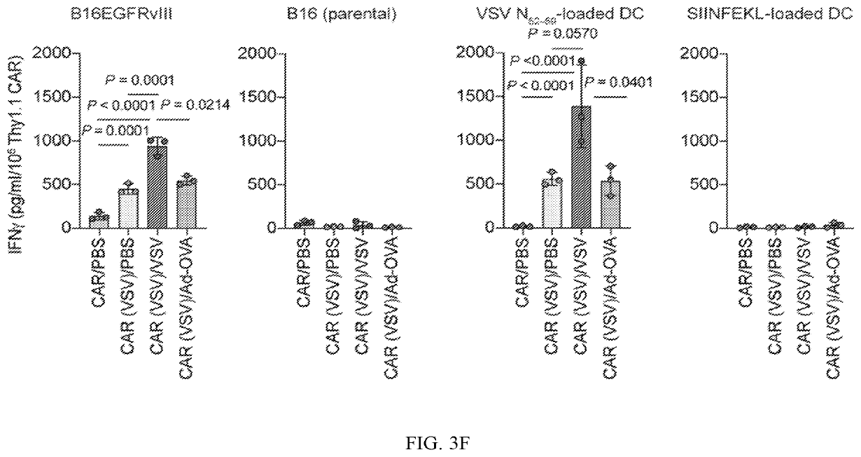

It has been shown that VSV, as well as other OVs, can be loaded onto CD8 T cells in vitro and subsequently carried as hitchhikers to tumors in vivo with greater efficiency than with intravenous injection of virus alone (Cole et al., Nat. Med., 11:1073-1081 (2005); and Qiao et al., Gene Ther., 15:604-616 (2008)). On the basis of these data, it was evaluated whether the generation of DS CAR T cells could be optimized by enhancing the codelivery of both virus and CAR T cells to secondary lymphoid organs for presentation of viral antigens to the CAR T cells. Although intravenously administered VSV was readily detected in the lymph node (LN), higher titers were detected after the in vitro loading of CAR T cells with the virus 2 days after delivery (FIG. 3A). This in vitro loading of CAR T cells with virus also led to higher titers of VSV in subcutaneous tumors compared to intravenous delivery alone. In addition to improved viral delivery to LNs and tumors, greater numbers of CAR T were recovered from LNs if cells had been preloaded with VSV in vitro (FIG. 3B). The improved LN localization of virus-loaded CAR T cells was mimicked by pretreatment of CAR T cells with lipopolysaccharide (LPS) or (Polyinosinic-polycytidylic acid (poly(I:C)) but not CpG, suggesting that virus loading may be triggering CAR T cell activation and altered trafficking through Toll-like receptors (TLRs) 3 or 4, but not 9 (FIG. 3B). Neither VSV or reovirus loading, nor LPS or poly(I:C) treatment of CAR T cells enhanced CAR T cell trafficking to the tumor, although prior treatment with CpG was able to increase CAR T cell recovery from the tumor by about threefold (FIG. 3B).

Consistent with the results in FIG. 2, DS CAR T cells generated by in vitro CAR T cell loading were also more functional than CAR T administered as a monotherapy. CAR T cells isolated from splenocytes of mice treated 15 days previously with CAR T cells alone secreted low but detectable IFNγ when cocultured with B16EGFRvIII target cells, but not when cocultured with parental B16 cells (FIG. 3C, left versus middle left). CAR T cells from mice treated with VSV-mIFNβ intravenously 5 days after CAR T cell transfer were more active against B16EGFRvIII targets than CAR T cells from mice treated with no virus, and CAR T cells were reactive against the VSV N₅₂₋₅₉ peptide, but not the irrelevant SIINFEKL (SEQ ID NO:1) peptide (FIG. 3C, middle right versus right). However, CAR T cells recovered from mice treated with in vitro VSV-mIFNβ-loaded CAR T cells secreted more IFNγ in response to both CAR antigen and VSV antigen than CAR T from either of the other two groups (FIG. 3C, left and middle right). Together, these data show that in vitro loading of CAR T cells with virus enhanced both CAR T cell and virus delivery to the LN for priming of DS CAR T cells and was a more effective way to generate DS CAR T cells in vivo in nonpreconditioned mice than by physically and temporally separating CAR T and virus administration.

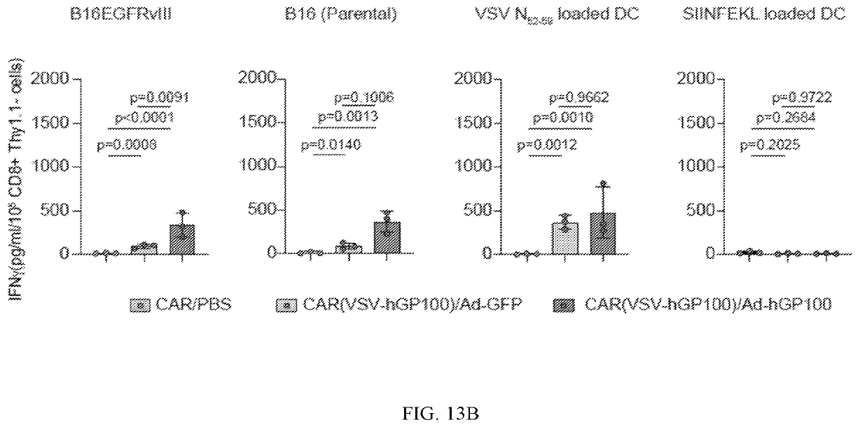

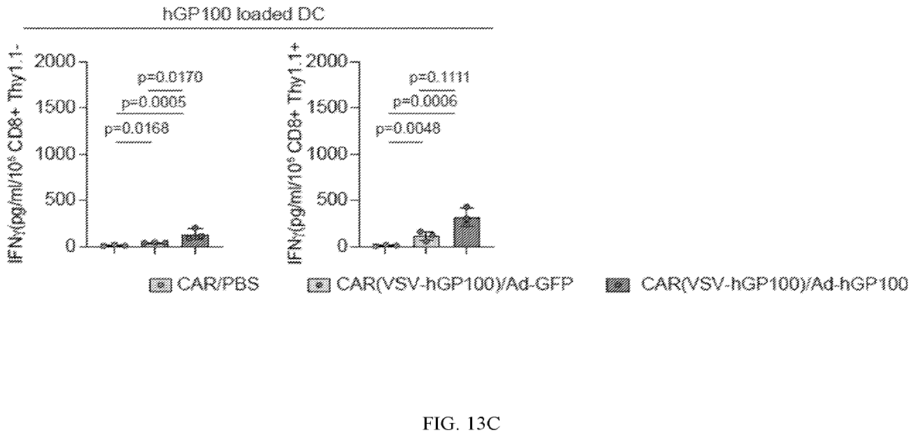

Since in vitro loading of CAR T cells with VSV could enhance virus delivery to tumors and promote the expansion of DS CAR T cells with greater function, it was tested whether this strategy could be exploited therapeutically. It was further evaluated whether a second in vivo boost with virus would restimulate memory CAR T cells with viral TCR specificity. Consistent with the enhanced function of DS CAR T cells over CAR T cells alone, in vitro virus-loaded CAR T cells conferred a survival advantage compared to either unloaded CAR T cells or intravenous virus alone against subcutaneous B16EGFRvIII tumors (FIGS. 3D and 3E). However, treatment with virus-loaded CAR T cells alone was insufficient to cure any mice long term in this regimen. In contrast, a boost with intravenous VSV-mIFNβ was able to restimulate DS CAR T cell activity in vivo, leading to tumor protection in six of seven mice up to 60 days. Antitumor efficacy was dependent on restimulation of VSV-specific CAR T cells because a boost with a different virus expressing an irrelevant antigen (replication defective Adenovrius (Ad) vector encoding the OVA gene (Ad)-OVA) was no more effective than treatment with VSV-loaded CAR T cells with no boost. CAR T cells were recovered from spleens of mice either at euthanasia due to tumor size or at the termination of the experiment at day 60, and their function was assessed in vitro. CAR T cells from mice treated with virus-loaded CAR T cells were more active against B16EGFRvIII targets than CAR T cells from mice treated with CAR T cells alone (FIG. 3F). However, a boost with VSV-mIFNβ, but not with a heterologous virus (Ad-OVA), enhanced the activity of the CAR T cells against B16EGFRvIII targets. Reactivity of these CAR T cells against the immunodominant VSV N₅₂₋₅₉ peptide mirrored the reactivity against the EGFRvIII targets, confirming their dual specificity after coadministration of CAR T with virus in vivo. Endogenous CD8 T cells (Thy1.1⁻ non-CAR T cells) showed low but detectable reactivity against B16 tumor cells (irrespective of expression of EGFRvIII), as well as against VSV N₅₂₋₅₉ if the CAR T cells were in vitro loaded with VSV (FIG. 13A). These data indicated that DS CAR T cell therapy was able to induce priming of endogenous T cells against both viral and tumor-associated antigens in vivo.

### Virus Loading and Boosting DS CAR T Cell Therapy are not Dependent on Virus or Tumor Type

CAR T cells preloaded with oncolytic reovirus led to improved survival compared to unloaded CAR T cells in the same subcutaneous B16EGFRvIII mouse model (FIGS. 4A and 4B), similar to the results with VSV-loaded CAR T cells. In addition, a systemic boost with reovirus, but not with a heterologous virus, VSV, reactivated the virus-specific CAR T cells, leading to tumor cures in six of seven mice out to day 60. In vitro reovirus loading on CAR T cells also increased the in vivo persistence and expansion of CAR T cells, which could be detected at endpoint in comparison to unloaded CAR T cells (FIG. 4C). An additional systemic boost with reovirus, but not with VSV, induced further expansion of CAR T cell numbers, suggesting that this expanded population is reovirus specific.

A similar systemic regimen of CAR T cells preloaded with reovirus, along with subsequent boosting with reovirus, also cured >80% of mice of aggressive CT2A EGFRvIII brain tumors (FIGS. 4D and 4E), indicating that this strategy was not dependent on the location of the tumor being treated. The systemic boost with reovirus also greatly expanded the number of CAR T cells in the brains of tumor-cured mice compared to mice treated with CAR T cells alone or with CAR T cells loaded with reovirus but not treated with a subsequent virus boost (FIG. 4F). Unlike the subcutaneous melanoma model (FIG. 4C), in the case of these intracranial tumors, CAR T cells loaded with reovirus, but not boosted in vivo, were not detected at higher frequency than unloaded CAR T cells alone (FIG. 4F), which may be explained by different properties of the subcutaneous and intracranial tumor locations. When restimulated in vitro, splenocytes from mice treated with reovirus-loaded CAR T cells and a reovirus boost consistently secreted the highest amount of IFNγ upon coculture with CT2AEGFRvIII cells (FIG. 4G). Moreover, splenocytes from mice that survived long term in various groups in the experiment of FIG. 4E secreted more IFNγ when restimulated in vitro with CAR target cells.

Unlike virus-naïve mice used in these experiments, human patients with cancer have TCR repertoires that recognize many viral reactivities through natural exposure or vaccination. It was tested how generating CAR T cells from a pool of T cells in which preexisting antiviral memory cells were present would affect the efficacy of DS CAR T cell therapy in CT2AEGFRvIII intracranial tumor-bearing mice (FIG. 4H). When CAR T cells were generated from mice previously immunized with reovirus, preloading with reovirus enhanced survival compared to mice that received reovirus-preloaded CAR T cells prepared from virus-naïve donor mice (FIG. 4I). In this scenario, a further boost with virus did not significantly improve the efficacy of the CAR_{IMMUNE} (Reo) treatment because the initial therapy was so effective. These data suggest that reactivation of virus-specific memory CAR T cells by in vivo delivery and presentation of preloaded virus directly boost CAR T_{IMMUNE} cell activity in the same way the virus boosts activity 15 days after the generation of DS CAR T cells from CAR T_{NAÏVE} (Reo).

### DS CAR T Cells Target Virally Encoded MHC-Restricted Tumor Antigens

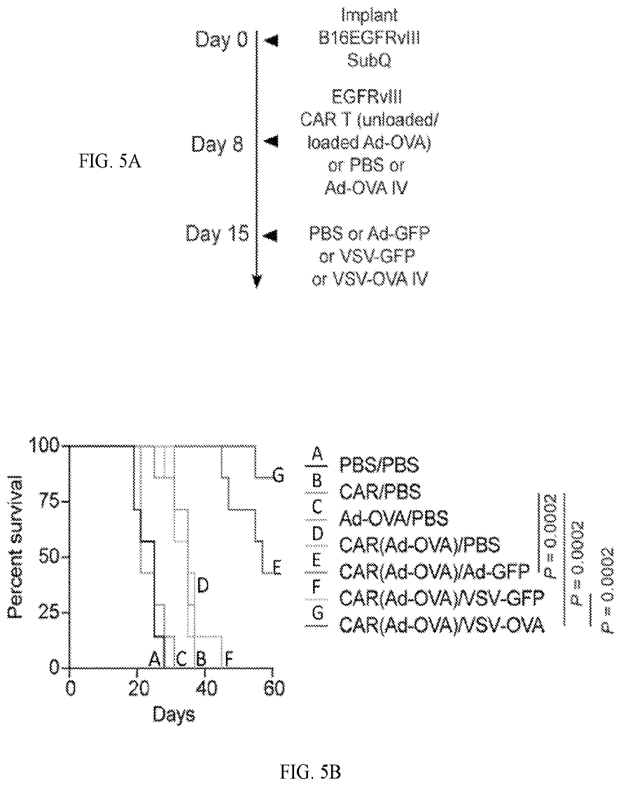

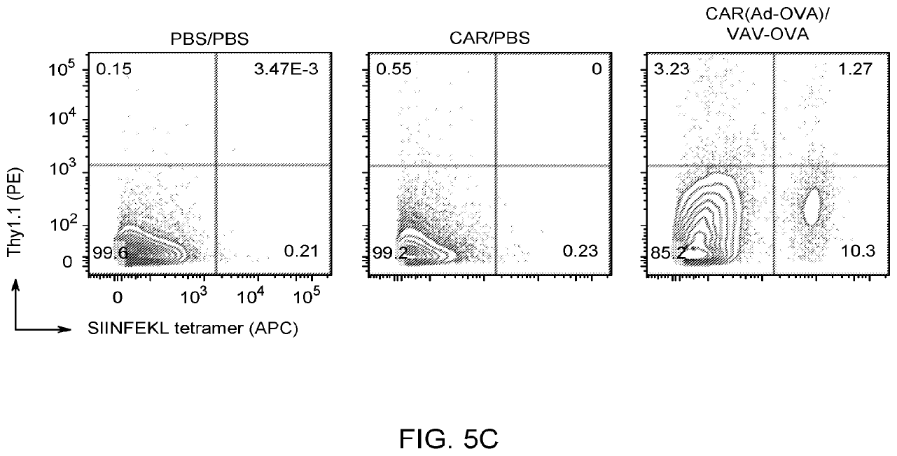

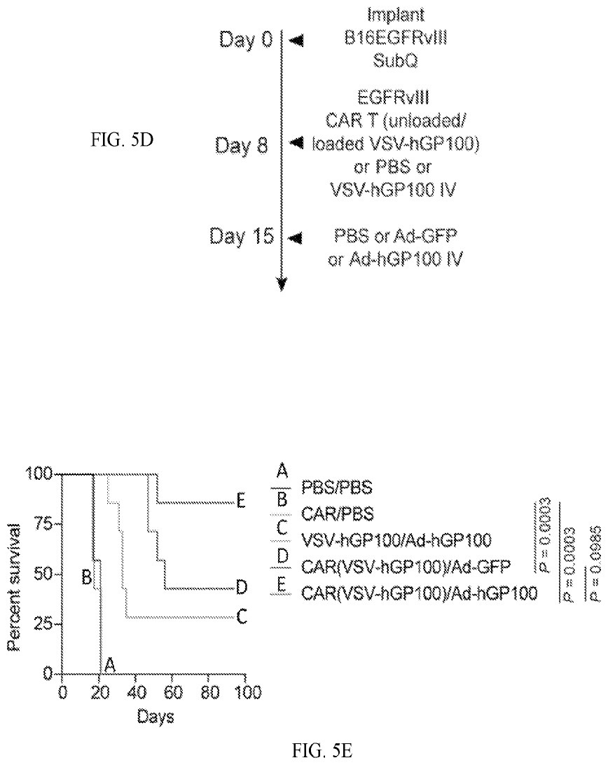

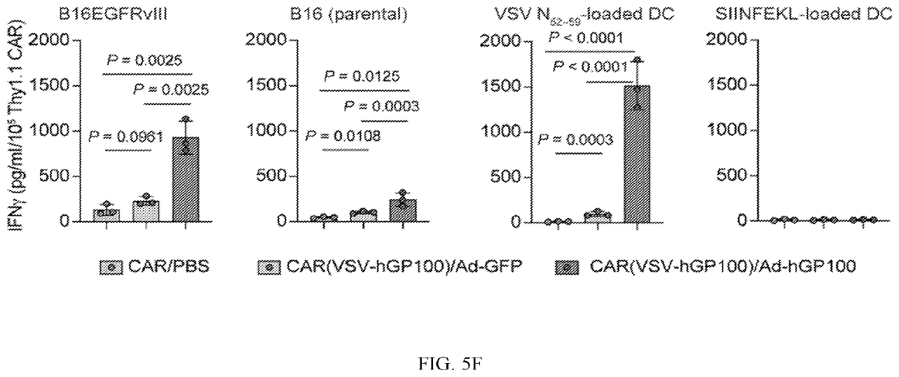

It was evaluated whether DS CAR T cells could also be primed through their TCR against antigens encoded by viruses and against the viral epitopes themselves. To test this, mice bearing B16EGFRvIII tumors were treated with CAR T cells loaded with an Ad-OVA (FIG. 5A). Unlike with either VSV or reovirus, CAR T cell loading with the Ad-OVA vector did not enhance CAR T cell efficacy compared to unloaded CAR T cells (FIG. 5B), possibly due to a different ability of Ad vectors, which do not infect murine cells efficiently, to stimulate TLR signaling in murine CAR T cells. However, the therapeutic efficacy of CAR T cells loaded with Ad-OVA was enhanced by systemic boosting with the homologous virus (Ad-GFP), as was the case with CAR T cells loaded with either VSV or reovirus (FIG. 5B). As with VSV and reovirus, the efficacy of CAR loaded with Ad-OVA was not enhanced by systemic boosting with a heterologous virus such as VSV-GFP (FIG. 5B). In contrast, Ad-OVA-loaded CAR T cells were reactivated in vivo very effectively by a heterologous virus expressing the OVA antigen, VSV-OVA (FIG. 5B).

Using SIINFEKL (SEQ ID NO:1) tetramer staining, it was confirmed that in vitro loading of the CAR T cells with Ad-OVA followed by a systemic boost with VSV-OVA generated a population of DS CAR T cells (~1.3% of total CD8⁺ T cells in the spleen) (FIG. 5C). The expansion of OVA-specific CAR T cells was also associated with greater expansion of SIINFEKL (SEQ ID NO:1) nonspecific CAR T cells compared to mice treated with unloaded CAR T cells (FIG. 5C).

In the experiment of FIGS. 5A and 5B), OVA was not a tumor-associated antigen, meaning that it was not expressed in the B16EGFRvIII tumors, and acted strictly as an added immunogen for CAR T cell expansion and activation. The efficacy of CAR T cells loaded in vitro with oncolytic VSV encoding the human gp100 (hGP100) melanoma antigen to raise T cell responses against the murine tumor-associated GP100 antigen expressed in the B16EGFRvIII tumors was tested. Whereas treatment of mice bearing B16EGFRvIII tumors with CAR T cells alone was ineffective in the absence of preconditioning, as before, ex vivo loading with VSV-hGP100 enhanced therapeutic efficacy (FIG. 5E), due to a combination of altered function of the CAR T cells themselves and the delivery of oncolytic VSV to the tumors. However, providing a further systemic boost with Ad-hGP100 to the loaded CAR T cell arm generated additional therapy and cured six of seven mice of their tumors (FIG. 5E). At completion of the experiment on day 95, coculture of CAR T isolated from spleens of mice treated with CAR (VSV-hGP100) and boosted with Ad-hGP100 revealed subpopulations that were functionally specific for both the CAR antigen and the VSV N₅₂₋₅₉ epitope, but not for the irrelevant OVA-derived SIINFEKL (SEQ ID NO:1) antigen (FIG. 5F). A small population of CAR T that secreted IFNγ when restimulated with B16 parental tumor cells was observed, suggesting specificity for the MHC-restricted GP100 antigen (FIG. 5F). As in FIG. 13A, endogenous CD8 T cells (Thy1.1⁻, non-CAR T cells) showed low but detectable reactivity against B16 tumor cells irrespective of expression of EGFRvIII, and this was enhanced in mice that had received a boost with Ad-GP100 (FIG. 13B). Treatment with CAR T loaded with VSV-GP100 generated both CAR T cells (Thy1.1⁺) and endogenous T cells (Thy1.1⁻) with TCR specificity against the hGP100₂₅₋₃₃ H-2 D^{b}-restricted peptide, and this was further boosted by Ad-GP100 (FIG. 13C).

### Ex Vivo Virus Loading of CAR Tis Therapeutically Effective Even in Cases CAR Target Expression

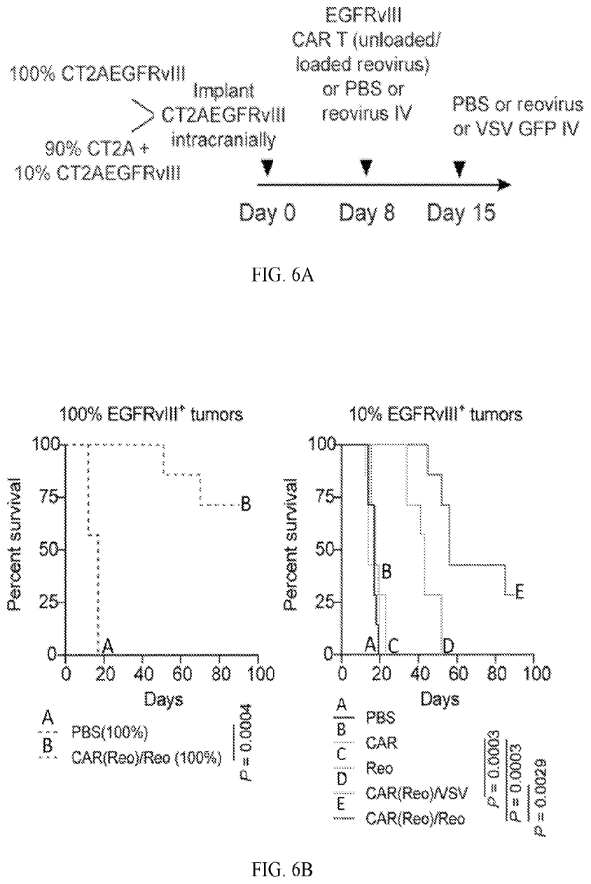

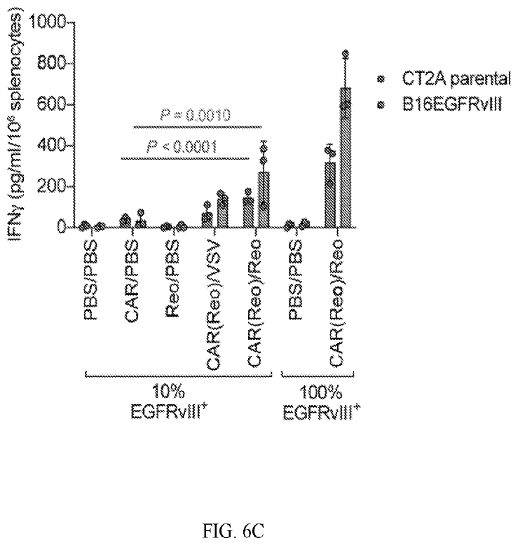

CAR antigen down-regulation or loss, or baseline heterogenous expression, can lead to therapeutic escape (Majzner et al., Cancer Discov., 8:1219-1226 (2018)). Therefore, it was evaluated whether the combined cytolytic and inflammatory oncolytic activities of both the CAR T and OV modalities would facilitate CAR T cell therapy even in tumors where CAR antigen expression was limiting. Mice bearing tumors composed entirely of target-expressing cells (100% CT2AEGFRvIII) or a mixed population (10% CT2AEGFRvIII+90% CT2A) were treated (FIG. 6A). CAR T cell therapy against brainstem tumors consisting of 90% CT2A cells and 10% CT2A-EGFRvIII cells was completely ineffective, as was systemically delivered reovirus (FIG. 6B). Loading of the CAR T cells with reovirus enhanced therapy but did not generate any cures consistent with both enhanced CAR T cell function and delivery of the oncolytic virus to the tumor. However, in vitro loading of the CAR T with reovirus combined with a further systemic boost with reovirus improved therapy still further and led to cures of two of seven mice (FIG. 6B).

The functionality of T cells at endpoint when restimulated with B16EGFRvIII cells or CT2A cells was also assessed. Splenocytes from mice treated with reovirus-loaded CAR T cells and a subsequent systemic boost with reovirus produced IFNγ when restimulated with CT2A cells (FIG. 6C), indicating that the treatment induced epitope spreading, leading to either endogenous T cell responses or DS CAR T cells reactive against the tumor. These splenocytes also contained CAR T cells that secreted the most IFNγ upon restimulation with B16EGFRvIII tumor cells (FIG. 6C). Comparatively, mice with tumors consisting entirely of CT2AEGFRvIII cells treated with reovirus-loaded CAR T combined with a further systemic boost with reovirus experienced better therapy than did mice bearing 10% CT2AEGFRvIII tumors (FIG. 6B). Splenocytes from mice with tumors consisting entirely of CT2AEGFRvIII cells showed improved responses against both endogenous CT2A-derived antigens and the CAR antigen (FIG. 6C).

### Human DS CAR T Cells can be Expanded in Distinct Functionality

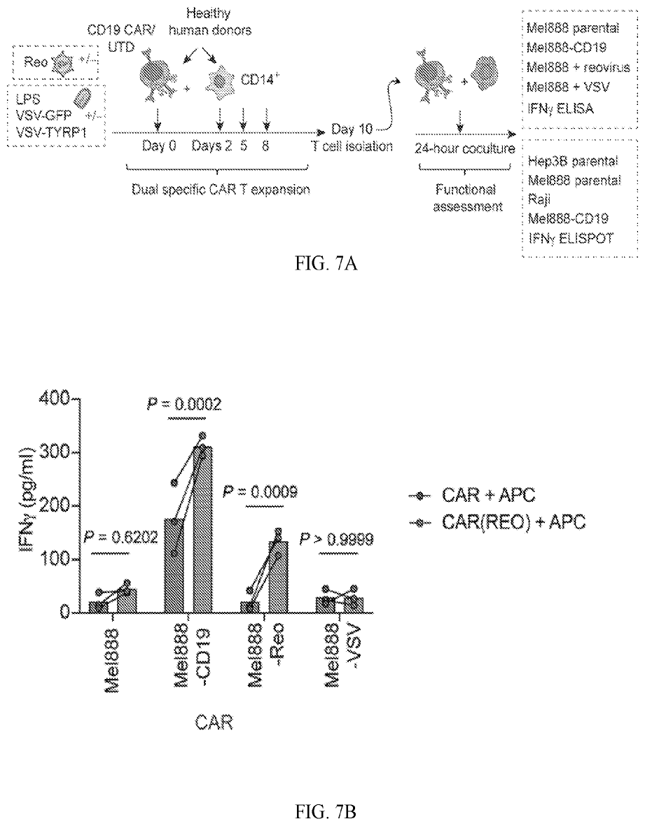

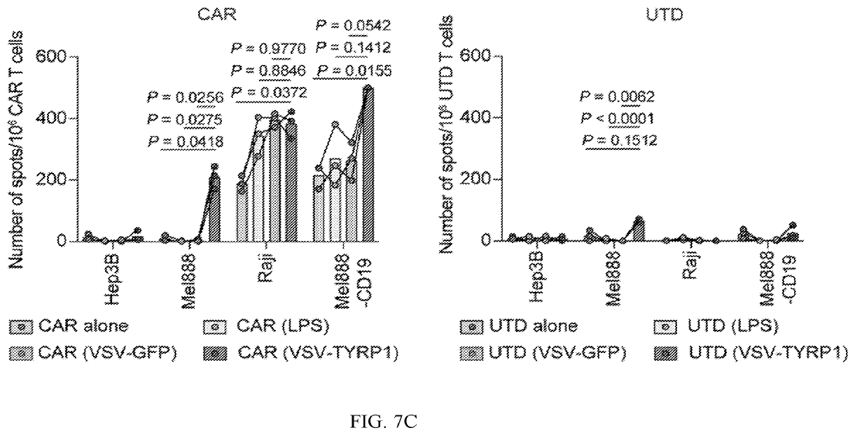

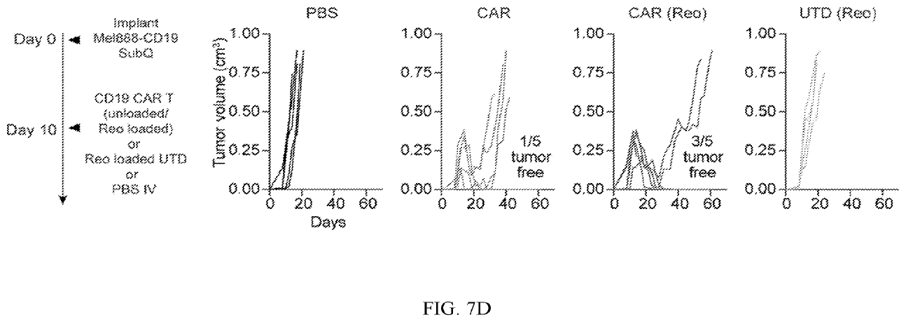

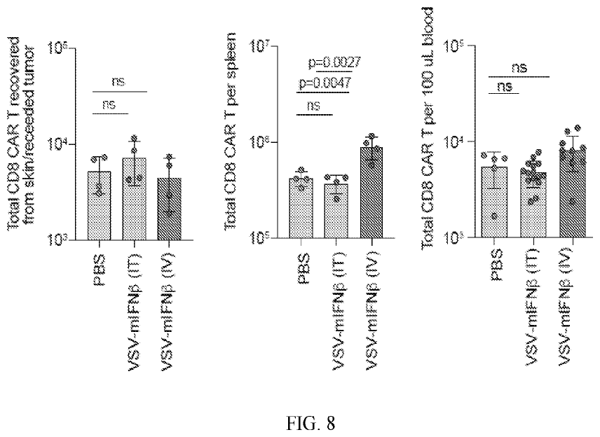

To investigate whether human virus-specific cells could be expanded and functionally validated in vitro, CD19-specific CAR T cells were loaded with virus and cocultured with autologous monocyte-derived dendritic cells as antigen-presenting cells (APCs) to expand the virus-specific T cell population. At the end of a 10-day priming phase, T cells were isolated and restimulated with CAR- or TCR-specific targets to test their functionality (FIG. 7A). Human CD19 CAR T cells expanded with unloaded APCs secreted IFNγ in response to Mel888 cells modified to express CD19, but not in response to parental tumor cells, or tumor cells preinfected with reovirus or VSV (FIG. 7B). CAR T cells loaded with reovirus secreted more IFNγ upon stimulation with CD19-expressing target cells than CAR T cells expanded without virus from the same donors. These reovirus-loaded CAR T cells also recognized reovirus-infected, but not VSV-infected, targets (FIG. 7B). These data show that in vitro priming of the CAR T cells was possible by hand-off of the loaded virus to APCs for presentation of viral epitopes to the CAR T cells to generate DS CAR T with TCR specificity for reovirus.

Similarly, the expansion and functional reactivity of untransduced T cells (UTD) or of CAR T cells loaded with VSV-GFP, VSV expressing the melanoma antigen tyrosinase-related protein (TYRP1), or LPS were tested. These T cells were cocultured with Hep3B cells (CD19⁻, TYRP1⁻), Mel888 cells (CD19⁻, TYRP1⁺), Raji cells (CD19⁺, TYRP1⁻), and Me1888 cells (CD19⁺, TYRP1⁺) using an IFNγ enzyme-linked immune absorbent spot (ELISpot). CD19 CAR T cells produced a similar number of spots when cocultured with either Raji or Mel888-CD19 cells (FIG. 7C). However, consistent with the murine data, human CD19 CAR T cells loaded with VSV-TYRP1 produced more IFNγ spots when cocultured with these same CAR target cells compared to unloaded CAR T cells. Moreover, IFNγ reactivity was observed against Mel888 parental cells, which express the TYPR1 antigen, only when CAR T cells were loaded with the VSV-TYRP1 virus. Loading the CAR T cells with a virus expressing the control green fluorescent protein (GFP) antigen improved the activity of the CAR T cells against CD19 target cells but did not prime the CAR for dual specificity against Mel888 melanoma targets (FIG. 7C). Also, consistent with the murine data, treating CAR T cells with LPS increased their activity against CD19 targets but did not confer any additional TCR specificity against melanoma targets. Together, these data show that virus loading leads to the priming of both mouse and human CAR T cells through their TCR against both viral antigens (FIGS. 7B and 7C) and against virus encoded MHC-restricted antigens (FIG. 7C).

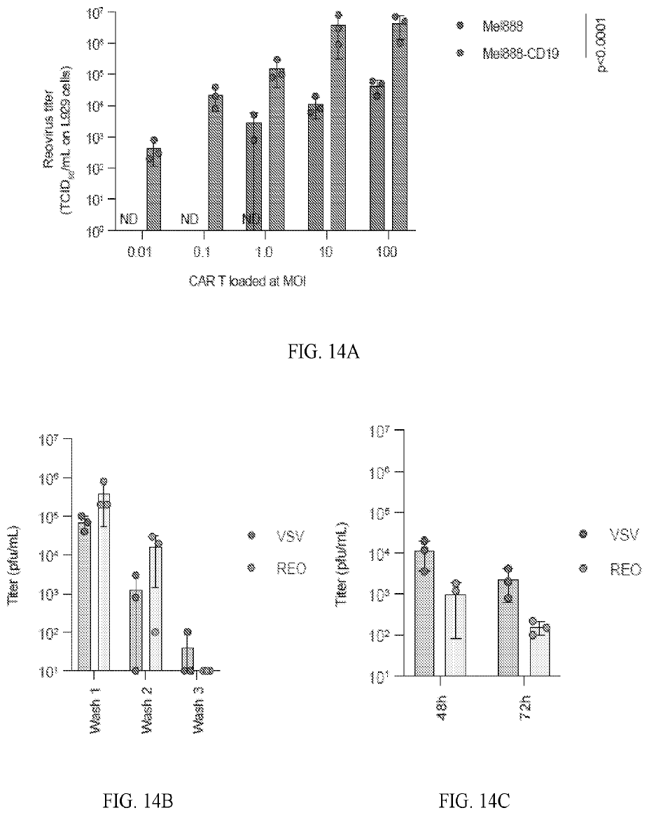

To maximize the use of viral carriage to tumors by in vitro loaded human CAR T cells, the optimal loading conditions by which virus could be made available to target tumor cells was tested. In vitro, virus-loaded anti-CD19 CAR T cells could transfer and release reovirus for infection of both CAR antigen-positive and -negative tumor cells (FIG. 14A). However, virus transfer from CAR T cells to recipient tumor cells was consistently higher at any given multiplicity of infection (MOI) of loading when the tumor cells expressed the CAR antigen. The optimal loading range of MOI for delivery to target cells was between 1.0 and 10. Loading of virus onto CAR T within this optimal range of MOI led to 90 to 99% of virus adhering to cells, as detected by nonadhered virus in the wash steps (FIG. 14B). low viral titers of reovirus or VSV were detected in the CAR after 48 and 72 hours (FIG. 14C), consistent with published observations that VSV-GFP did not productively infect CAR T cells (Evgin et al., Nat. Commun. 11:3187 (2020)).

Last, the therapeutic efficacy of reovirus-loaded human CAR T cells against human Mel888-CD19 tumors in NSG (NOD scid gamma) mice was tested. Using the in vitro loading conditions defined to be optimal in FIG. 14 (MOI 10), CAR T cells loaded with reovirus were more effective than either unloaded CAR T cells or virus-loaded UTD T cells (FIG. 7D). NSG mice treated intravenously with the equivalent virus dose as was loaded onto the CAR T cells (10⁸ plaque-forming units (pfu) per mouse) had to be euthanized by day 15 due to toxicity, showing that CAR T cell carriage of virus provides the additional benefits of both protecting systemically delivered virus and allowing for increased safety of delivery. Together with our murine immune competent studies, these data confirm that in vitro virus loading enhances the activity of CAR T cell therapy against solid tumors.

### Materials and Methods

### Study Design

These experiments were designed to evaluate the benefits of combining oncolytic viruses (OVs) such as VSV and reovirus with CAR T cell therapy against solid tumors in mice. Specifically, experiments were focused on examining how the native TCR in CAR-modified T cells were stimulated using OV-derived/encoded antigens. The immunocompetent murine studies were complemented by in vitro experiments and a xenograft model with human CAR T cells generated from healthy donor PBMCs.

### Cell Lines and Viruses

B16 murine melanoma cells, baby hamster kidney cells (BHK cells), L929 mouse fibroblasts, and 293T human embryonic kidney cells were originally obtained from the American Type Culture Collection and maintained in Dulbecco's modified Eagle's medium (DMEM; HyClone)+10% fetal bovine serum (FBS; Life Technologies). Cells were tested for mycoplasma using the MycoAlert Mycoplasma Detection Kit (Lonza). The B16EGFRvIII cell line was generated by retroviral transduction of B16 cells with the pBABE PURO vector encoding the murine EGFRvIII modified by the deletion of 500 amino acids from the intracellular domain of the protein. A clonally derived cell line was subsequently maintained in puromycin (1.25 μg/mL; Sigma-Aldrich). The CT2A and CT2AEGFRvIII cell line was maintained in DMEM+10% FBS.

VSV expressing murine IFNβ or GFP was rescued from the pXN2 cDNA plasmid and propagated on BHK cells at low multiplicity of infection. Twenty-four hours after infection, supernatant was harvested, filtered through a 0.22-μm filter to remove debris, and purified through a 10% sucrose cushion. Virus titers were determined by plaque assay on BHK cells. Wild-type reovirus type 3 (Dearing strain) was obtained from Oncolytics Biotech (Calgary, AB, Canada), and stock titers were measured by plaque assay on L929 cells.

### Mice

Female C57BL/6 (stock 000664) mice and 4-week-old NSG mice (stock 005557) were obtained from the Jackson Laboratory. All mice were obtained at 4 to 8 weeks of age and maintained in a specific pathogen-free BSL2 biohazard facility. Experimental mice were cohoused and exposed to a 12-hour light/12-hour dark cycle with unrestricted access to water and food. The ambient temperature was restricted to 20.6 to 26.1° C., and the room humidity ranged from 30 to 70%.

### Murine CAR T Cell Preparation

The EGFRvIII third-generation MSGV1 retroviral CAR construct contains the CD28, 4-1BB, and CD3z moieties, in tandem with the scFv derived from the human monoclonal antibody 139 and the marker Thy1.1. To prepare CAR T cells, splenocytes that were isolated from donor C57BL/6 mice were made into a single-cell suspension and cultured in RPMI (HyClone) supplemented with 10% FBS, 50 μM 2-mercaptoethanol (Sigma-Aldrich), 1% PenStrep (Corning), 1% Non-essential amino acids (NEAA) (Corning), 1% sodium pyruvate (Corning), human interleukin-2 (IL-2; 50 U/mL; Novartis), and concanavalin A (2.5 μg/mL; Sigma-Aldrich). In some experiments, CAR T cells were expanded using murine IL-21 (30 ng/ml), IL-15 (5 ng/mL), and IL-7 (10 ng/mL). Retroviral supernatant was produced from 293T cells cotransfected with the MSGV1 retroviral plasmid and the helper plasmid pCL Eco (Imgenex), and T cells were transduced on RetroNectin-coated plates (Takara) 2 days after stimulation. Cells were split 1 day after transduction and used for in vitro analysis or in vivo administration on day 4 or 5. Transduced cells were identified by the expression of Thy1.1.

### Human CAR T Cell Preparation

Peripheral blood mononuclear cells (PBMCs) were from healthy donor apheresis cones. Cells were isolated using Lympholyte-H density separation (Cedarlane) and cultured in AimV Media (Thermo Fisher Scientific) supplemented with 5% human AB serum (Sigma-Aldrich) and 1% PenStrep and stimulated with human IL-2 (100 U/mL) and anti-CD3 OKT3 antibody (50 ng/ml; BioLegend #317326). Forty-eight and 72 hours later, cells were transduced twice with lentiviral supernatant collected from 293T producer cells cotransfected with the anti-CD19 CAR and R8.91QV and pMD. G-packaging plasmids, on RetroNectin-coated plates (Takara). Cells were split every 2 days and collected 4 days after the second transduction for in vitro and in vivo experiments.

### In Vivo Studies

Mice were challenged subcutaneously with 2×10⁵ B16EGFRvIII cells in 100 μL of PBS (HyClone). Subcutaneous tumors were treated with VSV-mIFNβ, VSV-GFP, reovirus, Ad-OVA, and Ad-GFP delivered intratumorally in 50 μL of PBS or intravenously in 100 μL of PBS on day 9 or 15 depending on the schedule. Later, intravenous doses consisted of 1×10⁷ pfu of virus in 100 μL of PBS. CAR T cells were delivered intravenously by tail vein injection in 100 μL of PBS on day 4, 7, or 8 depending on the schedule. Tumors were measured using calipers three times per week, and mice were euthanized using CO2 when tumors reached 1.0 cm in diameter. For experiments requiring radiation, mice were subjected to 5-gray (Gy) total body irradiation 24 hours before CAR therapy. The survival endpoint was reached when the tumor size reached 1 cm in diameter. Tumor volume was calculated as follows: Volume=(Length×With²)/2.

CT2AEGFRvIII tumor cells were stereotactically implanted into the brainstem of C57BL/6 mice as described elsewhere (Caretti et al., Brain Pathol., 21:441-451 (2011)). Mice were monitored daily for gross neurologic symptoms including gait abnormalities, hunching, lethargy, seizures, paralysis, circling, and head tilt. Upon presentation of gross neurologic symptoms or poor body conditioning, mice were euthanized in accordance with IACUC standards.

### In Vitro Loading of CAR T Cells

CAR T cells were prepared as described above. On day 4 or 5 after transduction, cells were pelleted and washed twice in PBS. Pelleted cells were then incubated for 60 minutes at 4° C. with virus stock at a MOI of either 1 or 10. CAR T cell/virus pellets were then washed two to three times with PBS and resuspended at the appropriate cell density for in vivo administration.

In Vitro Sorting of CAR T Cells from Spleens

Single-cell suspensions from mouse spleens were prepared, and Thy1.1 (CD90.1) CAR T cells were isolated using CD90.1 MicroBeads (Miltenyi Biotec, order no. 130-121-273), as directed by the manufacturer before being used in in vitro restimulation assays.

### Generation of Murine Bone Marrow Dendritic Cells

Femurs were collected from C57/B16 mice, and bone marrow was flushed into RPMI media using a 25-gauge needle. Bone marrow was treated with Ammonium-Chloride-Potassium (ACK) Lysis Buffer, washed with serum-free RMP1, and then resuspended in RPMI supplemented with 10% FBS+1×penicillin/streptomycin+50 μM 2-mercaptoethanol supplemented with murine granulocyte-macrophage colony-stimulating factor (GM-CSF (20 ng/ml; Peprotech). Cells were seeded at 10⁶ cells per well in 2 mL of a 24-well plate. Media were replaced with fresh murine GM-CSF-containing media on day 3. Bone marrow derived dendritic cells (BMDCs) were collected on day 5.

### In Vitro Coculture/Restimulation Assays

For FIG. 2, CD8 CAR T cells were sorted on a BD fluorescence-activated cell sorting (FACS) Aria cell sorter as Thy1.1⁺ CD4. Cells were cocultured with tumor cells at an effector:target:nontarget ratio of 2:1:1. For the remaining figures, CD8⁺ T cells (endogenous or Thy1.1⁺ CD8⁺CAR T cells) isolated from mouse spleens were cocultured with target tumor cells (B16EGFRvIII or B16) pretreated for 24 hours with IFNγ or with murine in vitro matured dendritic cells preloaded for 24 hours with VSV N₅₂₋₅₉ or OVA-derived SIINFEKL (SEQ ID NO:1) peptides (5 μg per well) at an E:T ratio of 10:1. Forty-eight hours later, IFNγ was measured by ELISA (Mouse IFNγ ELISA Kit; OptEIA, BD Biosciences).

Virus-Loaded Human CAR T Cell Coculture with CD14+ Dendritic Cells

Fresh PBMCs were acquired from healthy donors to make human CAR T cells as described above. Autologous monocyte-derived dendritic cells were matured by isolating CD14⁺ cells by magnetic sorting (Miltenyi Biotec), followed by incubation with human GM-CSF (800 U/mL) and IL-4 (1,000 U/mL). On days 3 and 5, media were replaced with human GM-CSF (1600 U/mL) and IL-4 (1000 U/mL). On day 7, nonadherent cells were collected, washed with PBS, and resuspended in medium containing GM-CSF (800 U/mL), IL-4 (1000 U/mL), TNFα (1100 U/mL), IL-1B (1870 U/mL), IL-6 (1000 U/mL), and prostaglandin E2 (1 μg/mL). Two days later, dendritic cells were harvested for coincubation with activated CAR T cells loaded in vitro with virus (as described above) at a ratio of 1:1. After 10 days of coculture of dendritic cells with CAR T cells, CD3⁺ T cells were reisolated using a magnetic sorting kit (Miltenyi Biotec), and CD3⁺ T cells were isolated using a magnetic sorting kit (Miltenyi Biotec) and were immediately cocultured with IFNγ-pretreated (200 U/mL for 12 hours) tumor cell targets (parental Mel888; Mel888 stably transfected with human CD19; reovirus-infected Mel888 (MOI 0.1) or VSV-infected Mel888 cells (MOI 0.001)) at an E:T ratio of 10:1; 24 hours later, IFNγ was measured by ELISA (R&D). Alternatively, on day 10 after coculture of virus-loaded CAR T cells with dendritic cells, CD3⁻ T cells were reisolated by magnetic bead sorting, and 10⁶ T cells were cocultured with IFNγ-pretreated parental Hep3B, Mel888 cells, Raji, or Mel888-CD19 cells at an ET ratio of 10:1 in ELISpot wells (R&D Human IFN-gamma ELISpot kit, EL285). Forty-eight hours later, wells were developed, and the number of spots was counted.

### Flow Cytometry

Flow cytometry was performed on cultured cells or freshly explanted spleens, blood, tumors, or, where no palpable tumor existed, a 1.5 cm by 1.5 cm area of skin. Tumors and skin were weighed and treated with Liberase TL (Roche) and deoxyribonuclease I (DNase I) (Sigma-Aldrich) for 30 to 45 minutes at 37° C. Up to 30 mg of tumor or other tissue was stained and run on the flow cytometer. One hundred microliters of blood collected by submandibular vein bleed was subjected to red blood cell lysis and stained. Samples were fixed in 4% formaldehyde and analyzed using the BD FACSCantoX SORP flow cytometer using FACSDiva v8.0 software or the ZE5 Cell Analyzer using Everest v2.0 software in the Mayo Clinic Flow Cytometry Core. Data were analyzed using FlowJo version 10.5.

Mouse cells were stained with fluorochrome-conjugated antibodies against combinations of the following antigens: CD8B (BioLegend #140410/140416, clone 53-5.8; dilution, 1:500), CD4 (BioLegend #100451, clone GK1.5), Thy1.1 (BioLegend #202524, clone OX-7; dilution, 1:1000; or eBioscience #11-0900-85, cloneHIS51; dilution, 1:500), CD45 (BioLegend #103114, clone 30-F11; dilution, 1:500), CD45.2 (BioLegend #109828, clone 104; dilution, 1:250), CD127 (BioLegend #135007, clone A7R34; dilution, 1:100), CD62L (BioLegend #104431, clone MEL-14; dilution, 1:200), and Killer cell lectin-like receptor subfamily G member 1 (KLRG1) (BioLegend #138411/138419, clone 2F1/KLRG1; dilution, 1:200). TCR Vβ analysis was performed using the Anti-Mouse TCR VB Screening Panel (BD Pharmingen #557004; dilution, 1:5).

Cells were stained with the H-2K^{b} VSV NP₅₂₋₅₉ RGYVYQGL (SEQ ID NO:4) (Brilliant Violet 421-labeled tetramer) at a dilution of 1:500 or the H-2K^{b} chicken ova₂₅₇₋₂₆₄ SIINFEKL (SEQ ID NO:1) (APC-labeled tetramer) at a dilution of 1:150, which were obtained from the National Institutes of Health Tetramer Core Facility.

Intracellular staining was performed on cells stimulated for 5 hours in the presence of monensin and brefeldin A (dilution 1:1000; BD) and CD10⁷a (BioLegend #121625, clone 1D4B; 1:150). Intracellular cytokines were detected using the following fluorochrome-conjugated antibodies: IFNγ (BioLegend #505806, clone XMG1.2; dilution, 1:200), TNFα (BioLegend #506329 clone MP6-XT22; dilution, 1:200), and IL2 (BioLegend #503808 clone JES6-5H4; dilution, 1:200). The tumor cell killing assay was performed using target and nontarget cells stained with Cell Trace Violet or carboxyfluorescein diacetate succinimidyl ester (CFSE) (Thermo Fisher Scientific). Cell viability was determined using the Zombie fixable live dead viability dye (BioLegend #423106; dilution, 1:1500).

### Statistics

Data processing was performed in Microsoft Excel. Graphing and statistical analysis were performed with GraphPad Prism 9 software (GraphPad). Single comparisons were made using unpaired two-tailed t tests. Multiple comparisons were analyzed using one-way or two-way analyses of variance (ANOVAs) with a Tukey's or Sidak post hoc multiple-comparison test. Survival data were assessed using the log-rank Mantle Cox test with Bonferroni multiple-comparison correction. Group data are expressed as group means±SD.

### Example 2: Combination Therapy with Dual-Specific CAR T Cells and ICB Improves Efficacy Against Solid Tumors in Mice

This Example describes using CAR⁺ T cells and viruses in combination with immune checkpoint blockade (ICB) (e.g., administration of one or more immune checkpoint inhibitors) to treat cancer.

Dual specific (DS) CAR T cells (see, e.g., Example 1 and Evgin et al., Sci. Transl. Med. 14: eabn2231 (2022)) were administered to tumor bearing mice with CAR T cells loaded with a virus expressing a potential tumor antigen, and then the mice were administered a subsequent systemic boost with that antigen and anti-PD-1 antibodies.

C57Bl/6 mice were seeded subcutaneously with B16-EGFRvIII tumours on day 1. Mice bearing 5 day established intra-cranial (i.e.) B16-EGFRvIII tumors were treated with 10⁶ CAR T cells either left unloaded or loaded at an MOI or 5 with VSV-ova. A reduced number of CAR T cells were given in these experiments to enhance the chances of improving therapy with anti-PD-1. On day 12 mice were boosted IV with Ad-OVA (10⁸ pfu) or Ad-GFP. In addition, groups were given anti-PD-1 or control IgG for three consecutive days (days 12, 15, 17) (100 μg anti-mouse PD1 (clone RMP1-14 BioXCell #BE0146) or control rat IgG (Jackson ImmunoResearch #012-000-003)) per dose intraperitoneally (IP). Survival of mice with time are shown in Table 2 (n=8 per group).

TABLE 2
Number of Mice Tumor Free.

| | # Surviving Mice | |
|---|---|---|
| | (Day 60 post tumor challenge) | |
| Treatment | Experiment 1 | Experiment 2 |
| NO TREATMENT | 0/8 | 0/8 |
| CAR T No loading/No Boost//α-PD-1 | 0/8 | 0/8 |
| CAR T(VSV-ova) No Boost//α-PD-1 | 1/8 | 2/8 |
| CAR T(VSV-ova)/Ad-OVA Boost/Control IgG | 7/8 | 6/8 |
| CAR T(VSV-ova)/Ad-OVA Boost/α-PD-1 | 8/8 | 8/8 |

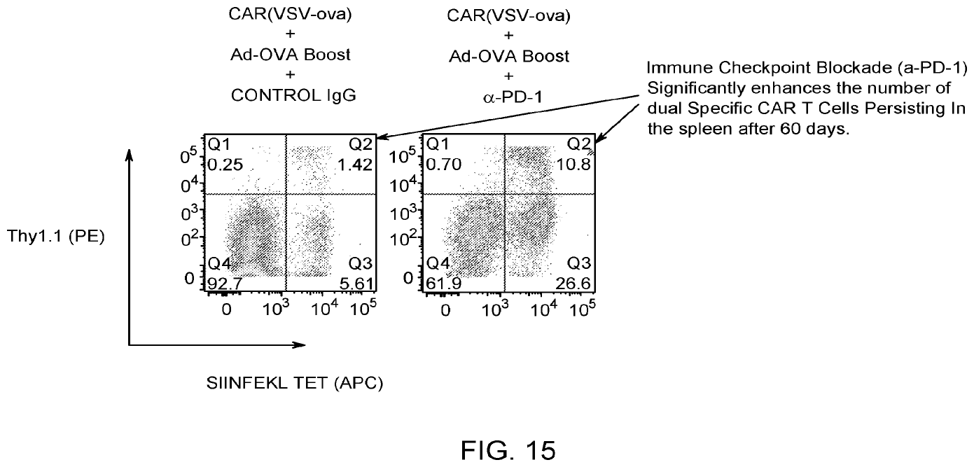

In two separate experiments, no statistically significant difference in anti-tumor therapy was observed between DS CAR T cells generated by loading with VSV-ova and boosted with OVA in the presence of anti-PD-1 antibodies compared to the same treatment with control IG-G. This lack of significance was largely because the control treatment for these experiments (CAR (VSV-ova)⁺ SIINFEKL (SEQ ID NO:1) boost⁺ Control IgG) was extremely effective at treating tumors (13 of 16 mice tumor free at day 60 post tumor challenge compared to 16 of 16 mice tumor free with anti-PD-1 antibodies). When spleens of mice surviving therapy at day 60 post tumor challenge were analyzed, a significant increase in the persistence of DS CAR T cells was observed in mice treated with anti-PD-1 compared to those treated with control IgG (FIG. 15).

Together these results demonstrate that immune checkpoint blockade can enhance functionality/persistence of DS CAR T cells and can improve anti-tumor therapy.

### Example 3: Treating Cancer with (a) CAR T Cells, (b) Virus, and (c) One or More Immune Checkpoint Inhibitors

CAR T cells expressing a CAR that can target (e.g., target and bind) a cancer antigen and viruses (e.g., oncolytic viruses) are systemically administered to mammals (e.g., mice or humans) having cancer. In some cases, CAR T cells and viruses are in separate compositions that are co-administered. In some cases, CAR T cells can be loaded (e.g., coated) with viruses and administered together as a single composition.

CAR T cells and viruses delivered to a mammal can generate dual-specific, T_{RM} CAR T cells that include an endogenous TCR specific for an antigen of the virus.

A boost (e.g., subsequent administration) of virus and one or more immune checkpoint inhibitors (e.g., anti-PD-1 antibodies) is provided systemically to mammals about 1 week after the co-administration of CAR T cells and viruses.

The systemic boost with virus and one or more immune checkpoint inhibitors (e.g., anti-PD-1 antibodies) can re-activate the dual-specific, T_{RM} CAR T cells in vivo via the endogenous TCR specific for an antigen of the virus and can increase persistence of the dual-specific, T_{RM} CAR T cells in vivo. Those re-activated dual-specific, T_{RM} CAR T cells can target (e.g., target and destroy) cells (e.g., cancer cells) presenting antigens recognized by the CAR present on the CAR T cells and/or can generate effector CAR T cells that can target (e.g., target and destroy) cells (e.g., cancer cells) presenting antigens recognized by the CAR present on those effector CAR T cells.

### Example 4: Treating Cancer with (a) CAR⁺ T Cells, (b) Virus Expressing Cancer Antigen, and (c) One or More Immune Checkpoint Inhibitors

CAR T cells expressing a CAR that can target (e.g., target and bind) a cancer antigen and viruses expressing an antigen of interest (e.g., oncolytic viruses expressing an antigen) are systemically administered to mammals (e.g., mice or humans) having cancer. In some cases, CAR T cells and viruses are in separate compositions that are co-administered. In some cases, CAR T cells can be loaded (e.g., coated) with viruses expressing an antigen and administered together as a single composition.

CAR T cells and viruses expressing an antigen of interest delivered to a mammal can generate dual-specific, T_{RM} CAR T cells that include an endogenous TCR specific for the antigen of interest.

A boost (e.g., subsequent administration) including (i) a composition that includes the antigen of interest that was expressed by the viruses and that lacks the viruses and (ii) one or more immune checkpoint inhibitors (e.g., anti-PD-1 antibodies) is provided systemically to mammals about 1 week after the co-administration of CAR T cells and viruses.

The systemic boost with (i) the composition that includes the antigen of interest and that lacks the viruses and (ii) the one or more immune checkpoint inhibitors (e.g., anti-PD-1 antibodies) can re-activate the dual-specific, T_{RM} CAR T cells in vivo via the endogenous TCR specific for the antigen of interest and can increase persistence of the dual-specific, T_{RM} CAR T cells in vivo. Those re-activated dual-specific, T_{RM} CAR T cells can target (e.g., target and destroy) cells (e.g., cancer cells) presenting antigens recognized by the CAR present on the CAR T cells and/or can generate effector CAR T cells that can target (e.g., target and destroy) cells (e.g., cancer cells) presenting antigens recognized by the CAR present on those effector CAR T cells.

### Example 5: Treating Cancer with (a) CAR⁺ T Cells, (b) Cancer Antigen, and (c) One or More Immune Checkpoint Inhibitors

CAR T cells expressing a CAR that can target (e.g., target and bind) a cancer antigen and an antigen are systemically administered to mammals (e.g., mice or humans) having cancer. In some cases, CAR T cells and antigens are in separate compositions that are co-administered. In some cases, CAR T cells can be loaded (e.g., coated) with the antigens and administered together as a single composition.

CAR⁺ T cells and antigens delivered to a mammal can generate dual-specific, T_{RM} CAR T cells that include an endogenous TCR specific for the antigens.

A boost (e.g., subsequent administration) of antigens and one or more immune checkpoint inhibitors (e.g., anti-PD-1 antibodies) is provided systemically to mammals about 1 week after the co-administration of CAR T cells and the antigens.

The systemic boost with the antigens and the one or more immune checkpoint inhibitors (e.g., anti-PD-1 antibodies) can re-activate the dual-specific, T_{RM} CAR T cells in vivo via the endogenous TCR specific for the antigens and can increase persistence of the dual-specific, T_{RM} CAR T cells in vivo. Those re-activated dual-specific, T_{RM} CAR T cells can target (e.g., target and destroy) cells (e.g., cancer cells) presenting antigens recognized by the CAR present on the CAR T cells and/or can generate effector CAR T cells that can target (e.g., target and destroy) cells (e.g., cancer cells) presenting antigens recognized by the CAR present on those effector CAR T cells.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a mammal having cancer, wherein said method comprises:
(a) administering a population of T cells with different endogenous T cell receptors (TCRs) to said mammal, wherein said T cells comprise a chimeric antigen receptor (CAR) that targets said cancer;
(b) administering a first antigenic composition to said mammal, wherein at least some of said T cells of said population form memory T cells within said mammal, wherein said memory T cells comprise said CAR and an endogenous TCR specific for an antigen of said first antigenic composition;
(c) administering a second antigenic composition comprising said antigen to said mammal, wherein said memory T cells are stimulated via their endogenous TCRs to form effector T cells comprising said CAR, and wherein said effector T cells reduce the number of cancer cells within said mammal; and
(d) administering an immune checkpoint inhibitor to said mammal.

2. The method of claim 1, wherein said mammal is a human.

3. The method of any one of claims 1-2, wherein said cancer is selected from the group consisting of a brain stem glioma, a pancreatic cancer, a bile duct cancer, a lung cancer, a skin cancer, a prostate cancer, a breast cancer, an ovarian cancer, a liver cancer, a colorectal cancer, a germ cell tumor, a hepatocellular carcinoma, a bowel cancer, a multiple myeloma, a lymphoma, and a leukemia.

4. The method of any one of claims 1-3, wherein said population of T cells with different endogenous TCRs comprises naïve T cells.

5. The method of claim 4, wherein said naïve T cells are selected from the group consisting of CD4⁻ T cells, CD8⁺ T cells, and any combination thereof.

6. The method of any one of claims 1-5, wherein said CAR can target a tumor-specific antigen on said cancer.

7. The method of claim 6, wherein said tumor-specific antigen is selected from the group consisting of cluster of differentiation 19 (CD19), CD22, CD20, GD2, EGFRvIII, mesothelin, IL-13RA, BCMA, CD138, NKG2-D, HER2/Neu, IL-13RA2, CD137, CD28, B7-H3 (CD276), CD16V, CA-125, MUC-1, epithelial tumor antigen, melanoma-associated antigen, mutated p53, mutated Ras, ERBB2, folate binding protein, HIV-1 envelope glycoprotein gp120, HIV-1 envelope glycoprotein gp41, CD123, CD23, CD30, CD56, c-Met, GD3, HERV-K, IL-11R alpha, kappa chain, lambda chain, CSPG4, and VEGFR2.

8. The method of any one of claims 1-7, wherein said first antigenic composition comprises a virus.

9. The method of claim 8, wherein said virus is an oncolytic virus.

10. The method of claim 8, wherein said virus is selected from group consisting of a vesiculovirus, a Maraba virus, a reovirus, an adenovirus, a vaccinia virus, a Newcastle disease virus, a poliovirus, a HSV virus, and a measles virus.

11. The method of any one of claims 8-10, wherein said endogenous TCR specific for said antigen is an endogenous TCR specific for an antigen of said virus.

12. The method of any one of claims 1-7, wherein said first antigenic composition comprises a virus expressing an antigen exogenous to said virus.

13. The method of claim 12, wherein said endogenous TCR specific for said antigen is an endogenous TCR specific for said antigen exogenous to said virus.

14. The method of any one of claims 1-7, wherein said first antigenic composition comprises an antigenic polypeptide.

15. The method of claim 14, wherein said endogenous TCR specific for said antigen is an endogenous TCR specific for said antigenic polypeptide.

16. The method of any one of claims 1-15, wherein said population of T cells with different endogenous TCRs and said first antigenic composition are administered to said mammal within from about 1 second to about 48 hours of each other.

17. The method of any one of claims 1-15, wherein said population of T cells with different endogenous TCRs and said first antigenic composition are administered to said mammal at the same time.

18. The method of any one of claims 1-15, wherein said population of T cells with different endogenous TCRs and said first antigenic composition are administered to said mammal are as a single composition.

19. The method of any one of claims 1-18, wherein said memory T cells are CD69⁺ and CD103⁻.

20. The method of any one of claims 1-19, wherein said memory T cells are selected from the group consisting of central memory T cells (T_{CM} cells), effector memory T cells (T_{EM} cells), terminally differentiated effector memory T cells (T_{EMRA} cells), and tissue resident memory T cells (T_{RM} cells).

21. The method of any one of claims 1-20, wherein said second antigenic composition is administered to said mammal at least 5 days after said administering of said population of T cells and said administering of said first antigenic composition.

22. The method of any one of claims 1-21, wherein said immune checkpoint inhibitor is selected from the group consisting of an anti-PD-1 antibody, an anti-PD-L1 antibody, an anti-CTL4A antibody, an anti-LAG-3 antibody, an anti-TIM-3 antibody, and an anti-CD200AR antibody.

23. The method of any one of claims 1-21, wherein said immune checkpoint inhibitor is selected from the group consisting of pembrolizumab, nivolumab, cemiplimab, ipilimumab, tremelimumab, durvalumab, dostarlimab, avelumab, atezolizumab, and relatlimab.

24. The method of any one of claims 1-21, wherein said immune checkpoint inhibitor is selected from the group consisting of BMS-8, BMS-37, BMS-202, BMS-230, BMS-242, BMS-1001, BMS-1166, SB415286, vorinostat, decitabine, entitostat, JQ1, BET151, GSK503, panobinostat, ACY-241, azacytidine, DB36, DB71, DB15, CVN, MGCD0103, SNDX-275, IMP32, BMS986016, TSR-022, Sym023, ATIK2a, and DZNep.

25. The method of any one of claims 1-24, wherein said second antigenic composition and said immune checkpoint inhibitor are administered to said mammal within from about 1 second to about 48 hours of each other.

26. The method of any one of claims 1-24, wherein said second antigenic composition and said immune checkpoint inhibitor are administered to said mammal at the same time.

27. The method of any one of claims 1-24, wherein said second antigenic composition and said immune checkpoint inhibitor are administered to said mammal are as a single composition.

28. The method of any one of claims 1-27, wherein said cancer cells within said mammal are reduced by at least 25 percent.

29. The method of any one of claims 1-28, wherein said method is effective to improve survival of said mammal.

30. The method of claim 29, wherein said survival of said mammal is improved by at least 25 percent.
